(19) United States
(12) Patent Application Publication
(10) Pub. No.: US 2009/0300800 A1
(43) Pub. Date: Dec. 3, 2009

(54) D-Amino Acid a Selectable Marker for Barley (Hordeum Vulgare L.) Transformation

(75) Inventors: Adelina Trifonova, Svaloev (SE); Beata Dedicova, Svaloev (SE); Luke Mankin, Raleigh, NC (US)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 11/991,918

(22) PCT Filed: Sep. 14, 2006
(86) PCT No.: PCT/EP2006/066343

§ 371 Date: Mar. 12, 2008

Related U.S. Application Data
(60) Provisional application No. 60/720,194, filed on Sep. 23, 2005.

(51) Int. Cl. A01H 5/00; C12N 5/04; A01N 43/36; A01N 37/00; C12N 15/82

(52) U.S. Cl. 800/293; 435/410; 504/287; 504/320; 800/278; 800/294; 800/300; 800/320

(57) ABSTRACT

The present invention relates to improved methods for the incorporation of DNA into the genome of a barley plant based on a D-alanine or D-serine selection. Preferably, the transformation is mediated by Agrobacterium.

### BACKGROUND OF THE INVENTION

1. Field of the Invention

The present invention relates to improved methods for the incorporation of DNA into the genome of a barley plant based on a D-alanine or D-serine selection. Preferably, the transformation is mediated by Agrobacterium.

2. Description of the Related Art

During the past decade, it has become possible to transfer genes from a wide range of organisms to crop plants by recombinant DNA technology. This advance has provided enormous opportunities to improve plant resistance to pests, diseases and herbicides, and to modify biosynthetic processes to change the quality of plant products. There have been many methods attempted for the transformation of monocotyledonous plants. “Biolistics” is one of the most widely used transformation methods. In the “biolistics” (microprojectile-mediated DNA delivery) method microprojectile particles are coated with DNA and accelerated by a mechanical device to a speed high enough to penetrate the plant cell wall and nucleus (WO 91/02071). The foreign DNA gets incorporated into the host DNA and results in a transformed cell. There are many variations on the “biolistics” method (Sanford 1990; Fromm 1990; Christou 1988; Sautter 1991).

While widely useful in dicotyledonous plants, Agrobacterium-mediated gene transfer has long been disappointing when adapted to use in monocots but has recently been adopted to monocot plants (Ishida et al. 1996; WO 95/06722; EP-A1 672 752; EP-A1 0 709 462).

An essential step in successful transformation experiment is selection of transgenic cells and later on transgenic tissues and plants by employing adequate selection system suitable in particular crop with public acceptance as well. Up till now basically three selection systems were successful for selecting transgenic barley. The most used system is involving the Streptomyces hygroscopisus bar gene for phosphinotricin acetyl transferase (Thompson et al. 1987) conferring resistance towards the herbicide Basta (Jähne et al. 1994; Wan and Lemaux 1994, Brinch-Petersen et al. 1996; Jensen et al. 1996; Koprek et al. 1996; Tingay et al. 1997; Patel et al. 2000, Trifonova et al. 2001; Travella et al. 2005) or PPT (U.S. Pat. No. 6,100,447). Another selection system uses the Esherichia coli hpt gene giving the resistance to the antibiotic hygromycine B (Elzen et al. 1985; Hagio et al. 1995) or nptI gene for neomycin phosphotransferase II following by selection using G418 (Fumatsiuki et al. 1995; U.S. Pat. No. 6,541,257). Studies by Brinch-Petersen et al. 1999 showed that lyC gene coding for lysine feedback desensitized aspartate kinase-III of the an E. coli mutant could be used as selectable marker for Agrobacterium-mediated transformation of barley as third selection system used for selecting transgenic barley.

Recently a new selection system based on D-amino acids was reported and demonstrated to be effective in Arabidopsis (WO 03/060133; Erikson et al. 2004). No use or adoption of this system in monocotyledonous plants such as barley has been described so far.

Multiple subsequent transformations of barley plants with more than one construct (necessary for some of the more complicated high-value traits and for gene stacking) is complicated due to the limited availability of suitable selection markers. This situation is becoming compounded as antibiotic resistance markers (such as hygromycin or kanamycin resistance) become less viable options as a result of tightened regulatory requirements and environmental concerns. Thus, selection systems for barley are essentially restricted to the bar selection system.

Accordingly, the object of the present invention is to provide an improved, efficient method for transforming barley plants based on D-amino acid selection. This objective is achieved by the present invention.

### SUMMARY OF THE INVENTION

This invention is describing the use of the D-amino acids for selecting transgenic barley plants in vitro when dsdA gene from E. coli or dao1 gene from Rhodotorula gracilis is introduced into barley cells via Agrobacterium mediated transformation. Expression of dsdA gene in transgenic barley cells enable the deamination of the D-serine, D-threonine or D-allothreonine used as selection compounds into pyruvate, water and ammonium. Expression of dao1 gene can be used for either positive or counter selection of transgenic barley tissues. Strategy depends on compound used for selection. D-serine and D-alanine are toxic for the plant tissues but if there are metabolized by DAAO non toxic product are maid. D-isoleucine and D-valine have low toxicity for the plant cells but are metabolized by DAAO into the toxic keto acid—3-oxopentanoate and 3-methyl-2-oxobutanoate (Erikson et al. (2004)).

A first embodiment of the invention relates to a method for generating a transgenic barley plant comprising the steps of
a) introducing into a barley cell or tissue a DNA construct comprising at least one first expression construct comprising a promoter active in said barley plant and operable linked thereto a nucleic acid sequence encoding an enzyme capable to metabolize D-alanine and/or D-serine,
b) incubating said barley cell or tissue of step a) on a selection medium comprising D-alanine and/or D-serine and/or a derivative thereof in a total concentration from about 1 mM to 100 mM for a time period of at least 5 days, and
c) transferring said barley cell or tissue of step b) to a regeneration medium and regenerating and selecting barley plants comprising said DNA construct.

Preferably, said DNA construct further comprises at least one second expression construct conferring to said barley plant an agronomic valuable trait.

Preferably, the enzyme capable to metabolize D-alanine or D-serine is selected from the group consisting of D-serine ammonia-lyases (EC 4.3.1.18), D-Amino acid oxidases (EC 1.4.3.3), and D-Alanine transaminases (EC 2.6.1.21). More preferably the enzyme capable to metabolize D-alanine or D-serine is selected from the group consisting of D-serine ammonia-lyases (EC 4.3.1.18), and D-Amino acid oxidases (EC 1.4.3.3). Even more preferably for the method of the invention, the enzyme capable to metabolize D-serine is selected from the group consisting of
i) the E. coli D-serine ammonia-lyase as encoded by SEQ ID NO: 2, and
ii) enzymes having the same enzymatic activity and an identity of at least 80% to the sequence as encoded by SEQ ID NO: 2, and
ii) enzymes encoded by a nucleic acid sequence capable to hybridize to the complement of the sequence described by SEQ ID NO: 1,
   and wherein selection is done on a medium comprising D-serine in a concentration from about 1 mM to 100 mM.

Also more preferably for the method of the invention, the enzyme capable to metabolize D-serine and D-alanine is selected from the group consisting of
i) the Rhodotorula gracilis D-amino acid oxidase as encoded by SEQ ID NO: 4, and
ii) enzymes having the same enzymatic activity and an identity of at least 80% to the sequence as encoded by SEQ ID NO: 4, and
iii) enzymes encoded by a nucleic acid sequence capable to hybridize to the complement of the sequence described by SEQ ID NO: 3,
   and wherein selection is done on a medium comprising D-alanine and/or D-serine in a total concentration from about 1 mM to 100 mM.

The promoter active in said barley plant is preferably an ubiquitin promoter, more preferably a monocot ubiquitin promoter, most preferably a maize ubiquitin promoter. Even more preferably, the ubiquitin promoter is selected from the group consisting of
a) sequences comprising the sequence as described by SEQ ID NO: 5, and
b) sequences comprising at least one fragment of at least 50 consecutive base pairs of the sequence as described by SEQ ID NO: 5, and having promoter activity in barley,
c) sequences comprising a sequence having at least 60% identity to the sequence as described by SEQ ID NO: 5, and having promoter activity in barley,
d) sequences comprising a sequence hybridizing to the sequence as described by SEQ ID NO: 5, and having promoter activity in barley.

The sequence described by SEQ ID NO: 5 is the core promoter of the maize ubiquitin promoter. In one preferred embodiment not only the promoter region is employed as a transcription regulating sequence but also a 5′-untranslated region and/or an intron. More preferably the region spanning the promoter, the 5′-untranslated region and the first intron of the maize ubiquitin gene are used, even more preferably the region described by SEQ ID NO: 6. Accordingly in another preferred embodiment the ubiquitin promoter utilized in the method of the invention is selected from the group consisting of
a) sequences comprising the sequence as described by SEQ ID NO: 6, and
b) sequences comprising at least one fragment of at least 50 consecutive base pairs of the sequence as described by SEQ ID NO: 6, and having promoter activity in barley,
c) sequences comprising a sequence having at least 60% identity to the sequence as described by SEQ ID NO: 6, and having promoter activity in barley,
d) sequences comprising a sequence hybridizing to the sequence as described by SEQ ID NO: 6, and having promoter activity in barley.

In one preferred embodiment of the invention the selection of step b) is done using about 1 mM to about 15 mM D-alanine or about 1 mM to about 30 mM D-Serine. The total selection time under dedifferentiating conditions is from about 3 to 4 weeks.

More preferably, the selection of step b) is done in two steps, using a first selection step for about 5 to about 35 days, then transferring the surviving cells or tissue to a second selection medium with essentially the same composition than the first selection medium for additional 5-35 days.

Various methods can be employed to introduce the DNA constructs of the invention into maize plants. Preferably, introduction of said DNA construct is mediated by a method selected from the group consisting of Rhizobiaceae mediated transformation and particle bombardment mediated transformation. More preferably, transformation is mediated by a Rhizobiaceae bacterium selected from the group of disarmed Agrobacterium tumefaciens or Agrobacterium rhizogenes bacterium strains. In another preferred embodiment the soil-borne bacterium is a disarmed strain variant of Agrobacterium rhizogenes strain K599 (NCPPB 2659). Such strains are described in U.S. provisional patent application No. 60/606,789, filed Sep. 2, 2004, hereby incorporated entirely by reference.

In one preferred embodiment of the invention the method of the invention comprises the following steps
a) isolating an immature embryo of a barley plant, and
b) co-cultivating said isolated immature embryo, which has not been subjected to a dedifferentiation treatment, with a bacterium belonging to genus Rhizobiaceae comprising at least one transgenic T-DNA, said T-DNA comprising at least one first expression construct comprising a promoter active in said barley plant and operably linked thereto a nucleic acid sequence encoding an enzyme capable to metabolize D-alanine and/or D-serine,
c) transferring the co-cultivated immature embryos to a recovering medium, said recovery medium lacking a phytotoxic effective amount of D-serine or D-alanine, and
d) inducing formation of embryogenic callus and selecting transgenic callus on a medium for comprising,
   i) an effective amount of at least one auxin compound, and
   ii) D-alanine and/or D-serine in a total concentration from about 1 mM to about 100 mM, and
e) regenerating and selecting plants containing the transgenic T-DNA from the said transgenic callus.

Preferably, said T-DNA further comprises at least one second expression construct conferring to said barley plant an agronomic valuable trait.

Preferably, the regeneration medium of step e. comprises
i) an effective amount of at least one cytokinin compound, and
ii) D-alanine and/or D-serine in a total concentration from about 1 mM to about 100 mM.

In said preferred method the selection of step d) is done using about 1 to about 15 mM D-alanine or about 1 to about 30 mM D-serine. More preferably, the selection of step d) is done in two steps, using a first selection step for about 5 to 35 days, then transferring the surviving cells or tissue to a second selection medium with essentially the same composition than the first selection medium for additional 5-35 days.

In said preferred recovery medium of step c) the effective amount of the auxin compound is preferably equivalent to a concentration of about 0.2 mg/l to about 6 mg/l 2,4-D or to a concentration of about 0.2 to about 6 mg/l Dicamba.

Virtually any barley plant can function as a source for the target material for the transformation. Preferably, said barley plant, immature embryo, cell or tissue is from a plant selected from the Hordeum family group of plants. More preferably, said barley cell or tissue or said immature embryo is (e.g., isolated) from a plant specie of the group consisting of Hordeum (H. vulgare subsp. Vulgare and Hordeum vulgare subsp. Spontaneum all diploid and tetraploid forms.),

The method of the invention, especially when used with D-Amino acid oxidases, can be advantageously combined with marker excision technology making use of the dual-function properties the D-amino acid oxidase. Thus, one embodiment of the invention relates to a method comprising the steps of:
i) transforming a barley plant cell with a first DNA construct comprising
   a) at least one first expression construct comprising a promoter active in said barley plant and operably linked thereto a nucleic acid sequence encoding a D-amino acid oxidase enzyme, wherein said first expression cassette is flanked by sequences which allow for specific deletion of said first expression cassette, and
   b) at least one second expression cassette suitable for conferring to said plant an agronomically valuable trait, wherein said second expression cassette is not localized between said sequences which allow for specific deletion of said first expression cassette, and
ii) treating said transformed barley plant cells of step i) with a first compound selected from the group consisting of D-alanine, D-serine or derivatives thereof in a phytotoxic concentration and selecting plant cells comprising in their genome said first DNA construct, conferring resistance to said transformed plant cells against said first compound by expression of said D-amino acid oxidase, and
iii) inducing deletion of said first expression cassette from the genome of said transformed plant cells and treating said plant cells with a second compound selected from the group consisting of D-isoleucine, D-valine and derivatives thereof in a concentration toxic to plant cells still comprising said first expression cassette, thereby selecting plant cells comprising said second expression cassette but lacking said first expression cassette.

The promoter active in barley plants and/or the D-amino acid oxidase are defined as above.

Another embodiment of the invention relates to a barley plant or cell comprising a promoter active in said barley plants or cells and operably linked thereto a nucleic acid sequence encoding an enzyme capable to metabolize D-alanine or D-serine, wherein said promoter is heterologous in relation to said enzyme encoding sequence. Preferably, the promoter and/or the enzyme capable to metabolize D-alanine or D-serine is defined as above. More preferably the barley plant is further comprising at least one second expression construct conferring to said barley plant an agronomically valuable trait. In one preferred embodiment the barley plant selected from the Hordeum vulgare ancestors. More preferably from a plant specie of the group consisting of Hordeum (H. vulgare subsp. Vulgare and Hordeum vulgare subsp. Spontaneum all diploid and tetraploid forms).

Other embodiments of the invention relate to parts, organs, cells, fruits, and other reproduction material of a barley plant of the invention. Preferred parts are selected from the group consisting of tissue, cells, pollen, ovule, anthers, inflosescences roots, leaves, seeds, microspores, and vegetative parts.

The methods and compositions of the invention can advantageously be employed in gene stacking approaches (i.e. for subsequent multiple transformations). Thus another embodiment of the inventions relates to a method for subsequent transformation of at least two DNA constructs into a barley plant comprising the steps of:
a) a transformation with a first construct said construct comprising at least one expression construct comprising a promoter active in said barley plants and operably linked thereto a nucleic acid sequence encoding an enzyme capable to metabolize D-alanine or D-serine, and
b) a transformation with a second construct said construct comprising a second selection marker gene, which is not conferring resistance against D-alanine or D-serine.

Preferably said second marker gene is conferring resistance against at least one compound select from the group consisting of phosphinothricin, glyphosate, sulfonylurea- and imidazolinone-type herbicides. More preferably, the marker gene is selected from the group of PAT or bar genes (e.g., from Streptomices higroscopicus or Streptomices). The promoter active in barley plants and/or the D-amino acid oxidase are defined as above.

Comprised are also the barley plants provided by such method. Thus another embodiment relates to a barley plant comprising
a) a first expression construct comprising a promoter active in said barley plant and operably linked thereto a nucleic acid sequence encoding an enzyme capable to metabolize D-alanine or D-serine, and
b) a second expression construct for a selection marker gene, which is not conferring resistance against D-alanine or D-serine.

Furthermore, the dsdA and dao gene provided hereunder can also be employed in subsequent transformations. Accordingly another embodiment of the invention relates to a method for subsequent transformation of at least two DNA constructs into a barley plant comprising the steps of:
a) a transformation with a first construct said construct comprising an expression construct comprising a plant promoter and operably linked thereto a nucleic acid sequence encoding an dsdA enzyme and selecting with D-serine, and
b) a transformation with a second construct said construct comprising an expression construct comprising a plant promoter and operably linked thereto a nucleic acid sequence encoding an dao enzyme and selecting with D-alanine.

The promoter active in barley plants and/or the D-amino acid oxidase are defined as above. Additional object of the invention relate to the model and the elite varieties of spring and winter barley. Preferred parts are selected from the group consisting of tissue, cells, pollen, anthers, ovule, microspores, inflorescence, roots, leaves, seeds, and meristematic tissues.

### DESCRIPTION OF THE DRAWINGS

FIG. 1: Constructs pRLM166

FIG. 2: Constructs pRLM167

FIG. 3: Constructs pRLM205

FIG. 4: Transgenic callus was expressing GUS
A) Barley callus vigorously grown on selection medium with D-serine;
B) GUS expression in transgenic barley callus.

FIG. 5: Transgenic regenerants selected on D-Serine:
A) In vitro rooted plants on selection medium;
B) Transgenic plant growing in soil.

### GENERAL DEFINITIONS

The teachings, methods, sequences etc. employed and described in the international patent applications WO 03/004659 (RECOMBINATION SYSTEMS AND A METHOD FOR REMOVING NUCLEIC ACID SEQUENCES FROM THE GENOME OF EUKARYOTIC ORGANISMS), WO 03/060133 (SELECTIVE PLANT GROWTH USING D-AMINO ACIDS), international patent application PCT/EP 2005/002735, international patent application PCT/EP 2005/002734, US provisional patent application No. 60/612,432 filed Sep. 23, 2004 are hereby incorporated by reference.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent, more preferably 5 percent up or down (higher or lower).

As used herein, the word “or” means any one member of a particular list and also includes any combination of members of that list.

“Agronomically valuable trait” include any phenotype in a plant organism that is useful or advantageous for food production or food products, including plant parts and plant products. Non-food agricultural products such as paper, etc. are also included. A partial list of agronomically valuable traits includes pest resistance, vigor, development time (time to harvest), enhanced nutrient content, novel growth patterns, flavors or colors, salt, heat, drought and cold tolerance, and the like. Preferably, agronomically valuable traits do not include selectable marker genes (e.g., genes encoding herbicide or antibiotic resistance used only to facilitate detection or selection of transformed cells), hormone biosynthesis genes leading to the production of a plant hormone (e.g., auxins, gibberllins, cytokinins, abscisic acid and ethylene that are used only for selection), or reporter genes (e.g. luciferase, glucuronidase, chloramphenicol acetyl transferase (CAT, etc.). Such agronomically valuable important traits may include improvement of pest resistance (e.g., Melchers 2000), vigor, development time (time to harvest), enhanced nutrient content, novel growth patterns, flavors or colors, salt, heat, drought, and cold tolerance (e.g., Sakamoto 2000; Saijo 2000; Yeo 2000; Cushman 2000), and the like. Those of skill will recognize that there are numerous polynucleotides from which to choose to confer these and other agronomically valuable traits.

As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The abbreviations used herein are conventional one letter codes for the amino acids: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid (see L. Stryer, Biochemistry, 1988, W. H. Freeman and Company, New York. The letter “x” as used herein within an amino acid sequence can stand for any amino acid residue.

The term “nucleic acid” refers to deoxyribonucleotides or ribonucleotides and polymers or hybrids thereof in either single- or double-stranded, sense or antisense form.

The phrase “nucleic acid sequence” as used herein refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”.

The term “nucleotide sequence of interest” refers to any nucleotide sequence, the manipulation of which may be deemed desirable for any reason (e.g., confer improved qualities), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product, (e.g., promoter sequence, polyadenylation sequence, termination sequence, enhancer sequence, etc.). A nucleic acid sequence of interest may preferably encode for an agronomically valuable trait.

The term “antisense” is understood to mean a nucleic acid having a sequence complementary to a target sequence, for example a messenger RNA (mRNA) sequence the blocking of whose expression is sought to be initiated by hybridization with the target sequence.

The term “sense” is understood to mean a nucleic acid having a sequence which is homologous or identical to a target sequence, for example a sequence which binds to a protein transcription factor and which is involved in the expression of a given gene. According to a preferred embodiment, the nucleic acid comprises a gene of interest and elements allowing the expression of the said gene of interest.

As used herein, the terms “complementary” or “complementarity” are used in reference to nucleotide sequences related by the base-pairing rules. For example, the sequence 5′-AGT-3′ is complementary to the sequence 5′-ACT-3′. Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. A “complement” of a nucleic acid sequence as used herein refers to a nucleotide sequence whose nucleic acids show total complementarity to the nucleic acids of the nucleic acid sequence.

The term “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

The term “chromosomal DNA” or “chromosomal DNA-sequence” is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), and in situ PCR.

Preferably, the term “isolated” when used in relation to a nucleic acid, as in “an isolated nucleic acid sequence” refers to a nucleic acid sequence that is identified and separated from at least one contaminant nucleic acid with which it is ordinarily associated in its natural source. Isolated nucleic acid is nucleic acid present in a form or setting that is different from that in which it is found in nature. In contrast, non-isolated nucleic acids are nucleic acids such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising SEQ ID NO:1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

As used herein, the term “purified” refers to molecules, either nucleic or amino acid sequences that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

A “polynucleotide construct” refers to a nucleic acid at least partly created by recombinant methods. The term “DNA construct” is referring to a polynucleotide construct consisting of deoxyribonucleotides. The construct may be single- or—preferably—double stranded. The construct may be circular or linear. The skilled worker is familiar with a variety of ways to obtain one of a DNA construct. Constructs can be prepared by means of customary recombination and cloning techniques as are described, for example, in Maniatis 1989, Silhavy 1984, and in Ausubel 1987.

The term “wild-type”, “natural” or of “natural origin” means with respect to an organism, polypeptide, or nucleic acid sequence, that said organism is naturally occurring or available in at least one naturally occurring organism which is not changed, mutated, or otherwise manipulated by man.

The term “foreign gene” refers to any nucleic acid (e.g., gene sequence) which is introduced into the genome of a cell by experimental manipulations and may include gene sequences found in that cell so long as the introduced gene contains some modification (e.g., a point mutation, the presence of a selectable marker gene, etc.) relative to the naturally-occurring gene.

The terms “heterologous nucleic acid sequence” or “heterologous DNA” are used interchangeably to refer to a nucleotide sequence, which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed. A promoter, transcription regulating sequence or other genetic element is considered to be “heterologous” in relation to another sequence (e.g., encoding a marker sequence or am agronomically relevant trait) if said two sequences are not combined or differently operably linked their natural environment. Preferably, said sequences are not operably linked in their natural environment (i.e. come from different genes). Most preferably, said regulatory sequence is covalently joined and adjacent to a nucleic acid to which it is not adjacent in its natural environment.

The term “transgene” as used herein refers to any nucleic acid sequence, which is introduced into the genome of a cell or which has been manipulated by experimental manipulations by man. Preferably, said sequence is resulting in a genome which is different from a naturally occurring organism (e.g., said sequence, if endogenous to said organism, is introduced into a location different from its natural location, or its copy number is increased or decreased). A transgene may be an “endogenous DNA sequence”, “an “exogenous DNA sequence” (e.g., a foreign gene), or a “heterologous DNA sequence”. The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

The term “transgenic” or “recombinant” when used in reference to a cell or an organism (e.g., with regard to a barley plant or plant cell) refers to a cell or organism which contains a transgene, or whose genome has been altered by the introduction of a transgene. A transgenic organism or tissue may comprise one or more transgenic cells. Preferably, the organism or tissue is substantially consisting of transgenic cells (i.e., more than 80%, preferably 90%, more preferably 95%, most preferably 99% of the cells in said organism or tissue are transgenic).

A “recombinant polypeptide” is a non-naturally occurring polypeptide that differs in sequence from a naturally occurring polypeptide by at least one amino acid residue. Preferred methods for producing said recombinant polypeptide and/or nucleic acid may comprise directed or non-directed mutagenesis, DNA shuffling or other methods of recursive recombination.

The terms “homology” or “identity” when used in relation to nucleic acids refers to a degree of complementarity. Homology or identity between two nucleic acids is understood as meaning the identity of the nucleic acid sequence over in each case the entire length of the sequence, which is calculated by comparison with the aid of the program algorithm GAP (Wisconsin Package Version 10.0, University of Wisconsin, Genetics Computer Group (GCG), Madison, USA) with the parameters being set as follows:

### Gap Weight: 12 Length Weight: 4

### Average Match: 2,912 Average Mismatch: −2,003

For example, a sequence with at least 95% homology (or identity) to the sequence SEQ ID NO: 1 at the nucleic acid level is understood as meaning the sequence which, upon comparison with the sequence SEQ ID NO: 1 by the above program algorithm with the above parameter set, has at least 95% homology. There may be partial homology (i.e., partial identity of less then 100%) or complete homology (i.e., complete identity of 100%).

The term “hybridization” as used herein includes “any process by which a strand of nucleic acid joins with a complementary strand through base pairing.” (Coombs 1994). Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids. As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl [see e.g., Anderson and Young, Quantitative Filter Hybridization, in Nucleic Acid Hybridization (1985)]. Other references include more sophisticated computations which take structural as well as sequence characteristics into account for the calculation of Tm.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4 to 6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of highly stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 80% to 90% homology to the first nucleic acid sequence, then another hybridization condition is said to be equivalent to the hybridization condition of interest if this other hybridization condition also results in hybridization of the first nucleic acid sequence with the other nucleic acid sequences that have from 80% to 90% homology to the first nucleic acid sequence.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above-listed conditions. Those skilled in the art know that whereas higher stringencies may be preferred to reduce or eliminate non-specific binding, lower stringencies may be preferred to detect a larger number of nucleic acid sequences having different homologies.

The term “gene” refers to a coding region operably joined to appropriate regulatory sequences capable of regulating the expression of the polypeptide in some manner. A gene includes untranslated regulatory regions of DNA (e.g., promoters, enhancers, repressors, etc.) preceding (upstream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, posttranscriptional cleavage and polyadenylation.

The terms “polypeptide”, “peptide”, “oligopeptide”, “polypeptide”, “gene product”, “expression product” and “protein” are used interchangeably herein to refer to a polymer or oligomer of consecutive amino acid residues.

The term “isolated” as used herein means that a material has been removed from its original environment. For example, a naturally-occurring polynucleotide or polypeptide present in a living animal is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment.

The term “genetically-modified organism” or “GMO” refers to any organism that comprises transgene DNA. Exemplary organisms include plants, animals and microorganisms.

The term “cell” or “plant cell” as used herein refers to a single cell. The term “cells” refers to a population of cells. The population may be a pure population comprising one cell type. Likewise, the population may comprise more than one cell type. In the present invention, there is no limit on the number of cell types that a cell population may comprise. The cells may be synchronized or not synchronized. A plant cell within the meaning of this invention may be isolated (e.g., in suspension culture) or comprised in a plant tissue, plant organ or plant at any developmental stage.

The term “organ” with respect to a plant (or “plant organ”) means parts of a plant and may include (but shall not limited to) for example roots, fruits, shoots, stem, leaves, anthers, sepals, petals, pollen, seeds, etc.

The term “tissue” with respect to a plant (or “plant tissue”) means arrangement of multiple plant cells including differentiated and undifferentiated tissues of plants. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissue may be in planta, in organ culture, tissue culture, or cell culture.

The term “plant” as used herein refers to a plurality of plant cells which are largely differentiated into a structure that is present at any stage of a plant's development. Such structures include one or more plant organs including, but are not limited to, fruit, shoot, stem, leaf, flower petal, etc.

The term “chromosomal DNA” or “chromosomal DNA-sequence” is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., PCR analysis, Southern blot analysis, fluorescence in situ hybridization (FISH), and in situ PCR.

The term “structural gene” as used herein is intended to mean a DNA sequence that is transcribed into mRNA which is then translated into a sequence of amino acids characteristic of a specific polypeptide.

The term “expression” refers to the biosynthesis of a gene product. For example, in the case of a structural gene, expression involves transcription of the structural gene into mRNA and—optionally—the subsequent translation of mRNA into one or more polypeptides.

The term “expression cassette” or “expression construct” as used herein is intended to mean the combination of any nucleic acid sequence to be expressed in operable linkage with a promoter sequence and—optionally—additional elements (like e.g., terminator and/or polyadenylation sequences) which facilitate expression of said nucleic acid sequence.

“Promoter”, “promoter element,” or “promoter sequence” as used herein, refers to the nucleotide sequences at the 5′ end of a nucleotide sequence which direct the initiation of transcription (i.e., is capable of controlling the transcription of the nucleotide sequence into mRNA). A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription. Promoter sequences are necessary, but not always sufficient, to drive the expression of a downstream gene. In general, eukaryotic promoters include a characteristic DNA sequence homologous to the consensus 5′-TATAAT-3′ (TATA) box about 10-30 bp 5′ to the transcription start (cap) site, which, by convention, is numbered +1. Bases 3′ to the cap site are given positive numbers, whereas bases 5′ to the cap site receive negative numbers, reflecting their distance from the cap site. Another promoter component, the CAAT box, is often found about 30 to 70 bp 5′ to the TATA box and has homology to the canonical form 5′-CCAAT-3′ (Breathnach 1981). In plants the CAAT box is sometimes replaced by a sequence known as the AGGA box, a region having adenine residues symmetrically flanking the triplet G(or T)NG (Messing 1983). Other sequences conferring regulatory influences on transcription can be found within the promoter region and extending as far as 1000 bp or more 5′ from the cap site. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

Regulatory Control refers to the modulation of gene expression induced by DNA sequence elements located primarily, but not exclusively, upstream of (5′ to) the transcription start site. Regulation may result in an all-or-nothing response to environmental stimuli, or it may result in variations in the level of gene expression. In this invention, the heat shock regulatory elements function to enhance transiently the level of downstream gene expression in response to sudden temperature elevation.

Polyadenylation signal refers to any nucleic acid sequence capable of effecting mRNA processing, usually characterized by the addition of polyadenylic acid tracts to the 3′-ends of the mRNA precursors. The polyadenylation signal DNA segment may itself be a composite of segments derived from several sources, naturally occurring or synthetic, and may be from a genomic DNA or an RNA-derived cDNA. Polyadenylation signals are commonly recognized by the presence of homology to the canonical form 5′-AATAA-3′, although variation of distance, partial “readthrough”, and multiple tandem canonical sequences are not uncommon (Messing 1983). It should be recognized that a canonical “polyadenylation signal” may in fact cause transcriptional termination and not polyadenylation per se (Montell 1983).

Heat shock elements refer to DNA sequences that regulate gene expression in response to the stress of sudden temperature elevations. The response is seen as an immediate albeit transitory enhancement in level of expression of a downstream gene. The original work on heat shock genes was done with Drosophila but many other species including plants (Barnett 1980) exhibited analogous responses to stress. The essential primary component of the heat shock element was described in Drosophila to have the consensus sequence 5′-CTGGAATNTTCTAGA-3′ (where N=A, T, C, or G) and to be located in the region between residues −66 through −47 bp upstream to the transcriptional start site (Pelham 1982). A chemically synthesized oligonucleotide copy of this consensus sequence can replace the natural sequence in conferring heat shock inducibility.

Leader sequence refers to a DNA sequence comprising about 100 nucleotides located between the transcription start site and the translation start site. Embodied within the leader sequence is a region that specifies the ribosome binding site.

Introns or intervening sequences refer in this work to those regions of DNA sequence that are transcribed along with the coding sequences (exons) but are then removed in the formation of the mature mRNA. Introns may occur anywhere within a transcribed sequence—between coding sequences of the same or different genes, within the coding sequence of a gene, interrupting and splitting its amino acid sequences, and within the promoter region (5′ to the translation start site). Introns in the primary transcript are excised and the coding sequences are simultaneously and precisely ligated to form the mature mRNA. The junctions of introns and exons form the splice sites. The base sequence of an intron begins with GU and ends with AG. The same splicing signal is found in many higher eukaryotes.

The term “operable linkage” or “operably linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. Operable linkage, and an expression cassette, can be generated by means of customary recombination and cloning techniques as described (e.g., in Maniatis 1989; Silhavy 1984; Ausubel 1987; Gelvin 1990). However, further sequences which, for example, act as a linker with specific cleavage sites for restriction enzymes, or as a signal peptide, may also be positioned between the two sequences. The insertion of sequences may also lead to the expression of fusion proteins. Preferably, the expression cassette, consisting of a linkage of promoter and nucleic acid sequence to be expressed, can exist in a vector-integrated form and be inserted into a plant genome, for example by transformation.

The term “transformation” as used herein refers to the introduction of genetic material (e.g., a transgene) into a cell. Transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzyme-linked immunosorbent assay (ELISA) which detects the presence of a polypeptide encoded by one or more of the transgenes. Alternatively, transient transformation may be detected by detecting the activity of the protein (e.g., [symbol] -glucuronidase) encoded by the transgene (e.g., the uid A gene) as demonstrated herein [e.g., histochemical assay of GUS enzyme activity by staining with X-gluc which gives a blue precipitate in the presence of the GUS enzyme; and a chemiluminescent assay of GUS enzyme activity using the GUS-Light kit (Tropix)]. The term “transient transformant” refers to a cell which has transiently incorporated one or more transgenes. In contrast, the term “stable transformation” or “stably transformed” refers to the introduction and integration of one or more transgenes into the genome of a cell, preferably resulting in chromosomal integration and stable heritability through meiosis. Stable transformation of a cell may be detected by Southern blot hybridization of genomic DNA of the cell with nucleic acid sequences which are capable of binding to one or more of the transgenes. Alternatively, stable transformation of a cell may also be detected by the polymerase chain reaction of genomic DNA of the cell to amplify transgene sequences. The term “stable transformant” refers to a cell which has stably integrated one or more transgenes into the genomic DNA (including the DNA of the plastids and the nucleus), preferably integration into the chromosomal DNA of the nucleus. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into plant cells in the form of plant viral vectors involving epichromosomal replication and gene expression which may exhibit variable properties with respect to meiotic stability. Transformation also includes introduction of genetic material into plant cells in the form of plant viral vectors involving epichromosomal replication and gene expression which may exhibit variable properties with respect to meiotic stability. Preferably, the term “transformation” includes introduction of genetic material into plant cells resulting in chromosomal integration and stable heritability through meiosis.

The terms “infecting” and “infection” with a bacterium refer to co-incubation of a target biological sample, (e.g., cell, tissue, etc.) with the bacterium under conditions such that nucleic acid sequences contained within the bacterium are introduced into one or more cells of the target biological sample.

The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogenes (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are herein incorporated by reference), and are commercially available (e.g., the helium gas-driven microprojectile accelerator (PDS-1000/He) (BioRad).

The term “microwounding” when made in reference to plant tissue refers to the introduction of microscopic wounds in that tissue. Microwounding may be achieved by, for example, particle bombardment as described herein.

The “efficiency of transformation” or “frequency of transformation” as used herein can be measured by the number of transformed cells (or transgenic organisms grown from individual transformed cells) that are recovered under standard experimental conditions (i.e. standardized or normalized with respect to amount of cells contacted with foreign DNA, amount of delivered DNA, type and conditions of DNA delivery, general culture conditions etc.) For example, when isolated immature embryos are used as starting material for transformation, the frequency of transformation can be expressed as the number of transgenic plant lines obtained per 100 isolated immature embryos transformed.

### DETAILED DESCRIPTION OF THE INVENTION

A first embodiment of the invention relates to a method for generating a transgenic plant
a) introducing into a barley cell or tissue a DNA construct comprising at least one first expression construct comprising a promoter active in said barley plant and operably linked thereto a nucleic acid sequence encoding an enzyme capable to metabolize D-alanine and/or D-serine,
b) incubating said barley cell or tissue of step a) on a selection medium comprising D-alanine and/or D-serine and/or a derivative thereof in a total concentration from about 1 mM to 100 mM for a time period of at least 5 days, and
c) transferring said barley cell or tissue of step b) to a regeneration medium and regenerating and selecting barley plants comprising said DNA construct.
   Preferably, said DNA construct is further comprising at least one second expression construct conferring to said barley plant an agronomically valuable trait.

The invention provides a new selection system for barley, which offers a minimized escape rate without interfering with embryogenic callus formation and high number of transgenic shoots regeneration in barley. In addition the selection has a potential advantage as a selective marker compare to the previously described antibiotic and/or herbicid based systems:
Defined phenotype of toxicity in in vitro.
No toxic for other organisms
No selective advantage for transgenic plants in the nature.
Naturally occurring in bacteria, fungi and animals.

The markers utilized herein after sequences from bacteria or yeast, which are commonly found in human and animal food or feed. In a preferred embodiment the markers and method provided herein allow for easy removal of the marker sequence. Furthermore, two protocols were provided herein which allows for efficient Agrobacterium—mediated transformation of barley. The plants obtained by the method of the invention were fertile with normal phenotype.

Further requirements of the method of the invention are described below. Accordingly, in one embodiment, the method of the invention comprises the introduction of a DNA construct as defined below, further comprises the selection as defined below and/or comprises furthermore the regeneration as defined below.

### 1. The DNA Construct of the Invention

In another embodiment of the invention the DNA construct comprising at least one first expression cassette comprising a promoter active in barley plants and operably linked thereto a nucleic acid sequence encoding an enzyme capable to metabolize D-alanine and/or D-serine.

In one embodiment, the method of the invention comprises the introduction of a second expression cassette, e.g. comprised in the first or in a second DNA construct. Thus, the second expression cassette can be introduced into said cells or tissues as part of a separate DNA construct, e.g. via co-transformation or e.g. a breeding or a cell fusion step.

Preferably, said DNA construct is further comprising at least one second expression construct conferring to said barley plant an agronomically valuable trait. In one embodiment the DNA construct is a T-DNA, more preferably a disarmed T-DNA (e.g., without neoplastic growth inducing properties).

The promoter active in barley plants and/or the D-amino acid oxidase are defined below in detail.

### 1.1 The First Expression Construct

In one embodiment of the invention the recombinant expression construct comprises a promoter active in barley plants and operable linked thereto a nucleic acid sequence encoding an enzyme capable to metabolize D-alanine or D-serine, wherein said promoter is heterologous in relation to said enzyme encoding sequence. The promoter active in barley plants and/or the D-amino acid oxidase are defined below in detail.

1.1.1 The Enzyme Capable to Metabolize D-Alanine or D-Serine

The person skilled in the art is aware of numerous sequences suitable to metabolize D-alanine and/or D-serine. The term “enzyme capable to metabolize D-alanine or D-serine” means preferably an enzyme, which converts and/or metabolizes D-alanine and/or D-serine with an activity that is at least two times (at least 100% higher), preferably at least three times, more preferably at least five times, even more preferably at least 10 times, most preferably at least 50 times or 100 times the activity for the conversion of the corresponding L-amino acid (i.e., D-alanine and/or D-serine) and—more preferably—also of any other D- and/or L- or achiral amino acid.

Preferably, the enzyme capable to metabolize D-alanine or D-serine is selected from the group consisting of D-serine ammonia-lyase (D-Serine dehydratases; EC 4.3.1.18; formerly EC 4. 2.1.14), D-Amino acid oxidases (EC 1.4.3.3), and D-Alanine transaminases (EC 2.6.1.21). More preferably, the enzyme capable to metabolize D-alanine or D-serine is selected from the group consisting of D-serine ammonia-lyase (D-Serine dehydratases; EC 4.3.1.18; formerly EC 4. 2.1.14), and D-Amino acid oxidases (EC 1.4.3.3).

The term “D-serine ammonia-lyase” (D-Serine dehydratases; EC 4.3.1.18; formerly EC 4. 2.1.14) means enzymes catalyzing the conversion of D-serine to pyruvate and ammonia. The reaction catalyzed probably involves initial elimination of water (hence the enzyme's original classification as EC 4.2.1.14), followed by isomerization and hydrolysis of the product with C—N bond breakage. For examples of suitable enzyme see http://www.expasy.org/enzyme/4.3.1.18.

The term “D-Alanine transaminases” (EC 2.6.1.21). means enzymes catalyzing the reaction of D-Alanine with 2-oxoglutarate to pyruvate and D-glutamate. D-glutamate is much less toxic to plants than D-Alanine. http://www.expasy.org/enzyme/2.6.1.21.

The term D-amino acid oxidase (EC 1.4.3.3; abbreviated DAAO, DAMOX, or DAO) is referring to the enzyme converting a D-amino acid into a 2-oxo acid, by—preferably—employing Oxygen (O₂) as a substrate and producing hydrogen peroxide (H₂O₂) as a co-product (Dixon 1965a,b,c; Massey 1961; Meister 1963). DAAO can be described by the Nomenclature Committee of the International Union of Biochemistry and Molecular Biology (IUBMB) with the EC (Enzyme Commission) number EC 1.4.3.3. Generally an DAAO enzyme of the EC 1.4.3.3. class is an FAD flavoenzyme that catalyzes the oxidation of neutral and basic D-amino acids into their corresponding keto acids. DAAOs have been characterized and sequenced in fungi and vertebrates where they are known to be located in the peroxisomes. In DAAO, a conserved histidine has been shown (Miyano 1991) to be important for the enzyme's catalytic activity. In a preferred embodiment of the invention a DAAO is referring to a protein comprising the following consensus motive:

[LIVM]-[LIVM]-H*-[NHA]-Y-G-x-[GSA]-[GSA]-x-G-x₅-G-x-A

wherein amino acid residues given in brackets represent alternative residues for the respective position, x represents any amino acid residue, and indices numbers indicate the respective number of consecutive amino acid residues. The abbreviation for the individual amino acid residues have their standard IUPAC meaning as defined above. D-Amino acid oxidase (EC-number 1.4.3.3) can be isolated from various organisms, including but not limited to pig, human, rat, yeast, bacteria or fungi. Example organisms are Candida tropicalis, Trigonopsis variabilis, Neurospora crassa, Chlorella vulgaris, and Rhodotorula gracilis. A suitable D-amino acid metabolising polypeptide may be an eukaryotic enzyme, for example from a yeast (e.g. Rhodotorula gracilis), fungus, or animal or it may be a prokaryotic enzyme, for example, from a bacterium such as Escherichia coli. For examples of suitable enzyme see http://www.expasy.org/enzyme/1.4.3.3.

Examples of suitable polypeptides which metabolise D-amino acids are shown in Table 1. The nucleic acid sequences encoding said enzymes are available form databases (e.g., under Genbank Acc.-No. U60066, A56901, AF003339, Z71657, AF003340, U63139, D00809, Z50019, NC_{—}003421, AL939129, AB042032). As demonstrated above, DAAO from several different species have been characterized and shown to differ slightly in substrate affinities (Gabler 2000), but in general they display broad substrate specificity, oxidatively deaminating all D-amino acids.

TABLE 1
Enzymes suitable for metabolizing D-serine and/or D-alanine.

| Enzyme | EC number | Example Source organism | Substrate |
|---|---|---|---|
| D-Serine dehydratase | EC 4.3.1.18 | P54555 Bacillus subtilis | D-Ser |
| (D-Serine ammonia | (originally | P00926 Escherichia coli. DSDA | D-Thr |
| lyase, D-Serine | EC | Q9KL72 Vibrio cholera. VCA0875 | D-allothreonine |
| deaminiase) | 4.2.1.14) | Q9KC12 Bacillus halodurans. | |
| D-Amino acid | EC 1.4.3.3 | JX0152 Fusarium solani | Most D-amino |
| oxidase | | O01739 Caenorhabditis elegans. | acid |
| | | O33145 Mycobacterium leprae. AAO. | |
| | | O35078 Rattus norvegicus (Rat) | |
| | | O45307 Caenorhabditis elegans | |
| | | P00371 Sus scrofa (Pig) | |
| | | P14920 Homo sapiens (Human) | |
| | | P14920 Homo sapiens (Human) | |
| | | P18894 Mus musculus (Mouse) | |
| | | P22942 Oryctolagus cuniculus | |
| | | P24552 Fusarium solani (subsp. pisi) | |
| | | P80324 Rhodosporidium toruloides | |
| | | (Yeast)(Rhodotorula gracilis) | |
| | | Q19564 Caenorhabditis elegans | |
| | | Q28382 Sus scrofa (pig) | |
| | | Q7SFW4 Neurospora crassa | |
| | | Q7Z312 Homo sapiens (Human) | |
| | | Q82MI8 Streptomyces avermitilis | |
| | | Q8P4M9 Xanthomonas campestris | |
| D-Amino acid | EC 1.4.3.3 | Q8PG95 Xanthomonas axonopodis | |
| oxidase | | Q8R2R2 Mus musculus (Mouse) | |
| | | Q8SZN5 Drosophila melanogaster | |
| | | Q8VCW7 Mus musculus (Mouse) | |
| | | Q921M5 Cavia parcellus (Guinea pig) | |
| | | Q95XG9 Caenorhabditis elegans | |
| | | Q99042 Trigonopsis variabilis | |
| | | Q9C1L2 Neurospora crassa | |
| | | Q9JXF8 Neisseria meningitidis | |
| | | Q9V5P1 Drosophila melanogaster | |
| | | Q9VM80 Drosophila melanogaster | |
| | | Q9X7P6 Streptomyces coelicolor | |
| | | Q9Y7N4 Schizosaccharomyces pombe | |
| | | (Fission yeast) SPCC1450 | |
| | | Q9Z1M5 Cavia porcellus (Guinea pig) | |
| | | Q9Z302 Cricetulus griseus | |
| | | U60066 Rhodosporidium toruloides, | |
| | | (Rhodotorula gracilis) strain TCC | |
| | | 26217 | |
| D-Alanine | EC-number | P54692 Bacillus licheniformis | D-Ala |
| transaminase | 2.6.1.21 | P54693 Bacillus sphaericus | D-Arg |
| | | P19938 Bacillus sp. (strain YM-1) | D-Asp |
| | | 007597 Bacillus subtilis | D-Glu |
| | | 085046 Listeria monocytogenes | D-Leu |
| | | P54694 Staphylococcus haemolyticus | D-Lys |
| | | | D-Met |
| | | | D-Phe |
| | | | D-Norvaline |

Especially preferred enzymes as well as preferred substrates are presented in bold letters
Especially preferred in this context are the dao1 gene (EC: 1.4. 3.3: GenBank Acc.-No.: U60066) from the yeast Rhodotorula gracilis (Rhodosporidium toruloides) and the E. coli gene dsdA (D-serine dehydratase (D-serine deaminase) [EC: 4.3. 1.18; GenBank Acc.-No.: J01603). The dao1 gene is of special advantage since it can be employed as a dual function marker (see international patent application PCT/EP 2005/002734).

In a preferred embodiment, the method of the invention comprises the use of the above mentioned preferred enzymes, in particular of the especially preferred enzymes together with the substrates indicated as preferred substrates.

Suitable D-amino acid metabolizing enzymes also include fragments, mutants, derivatives, variants and alleles of the polypeptides exemplified above. Suitable fragments, mutants, derivatives, variants and alleles are those, which retain the functional characteristics of the D-amino acid metabolizing enzyme as defined above. Changes to a sequence, to produce a mutant, variant or derivative, may be by one or more of addition, insertion, deletion or substitution of one or more nucleotides in the nucleic acid, leading to the addition, insertion, deletion or substitution of one or more amino acids in the encoded polypeptide. Of course, changes to the nucleic acid that make no difference to the encoded amino acid sequence are included.

For the method of the invention, the enzyme capable to metabolize D-alanine is selected from the group consisting of
i) the D-Alanine transaminase as shown in Table I, and
ii) enzymes having the same enzymatic activity and an identity of at least 80% (preferably at least 85%, more preferably at least 90%, even more preferably at least 95%, most preferably at least 98%) to an amino acid sequence of a D-Alanine transaminase as shown in Table I;
iii) enzymes having the same enzymatic activity and an identity of the encoding nucleic acid sequence of at least 80% (preferably at least 85%, more preferably at least 90%, even more preferably at least 95%, most preferably at least 98%) to a nucleic acid sequence of a D-Alanine transaminase as shown in Table I, and
iv) enzymes encoded by a nucleic acid sequence capable to hybridize to the complement of the sequence encoding the D-Alanine transaminase as shown in Table I,
   and wherein selection is done on a medium comprising D-alanine and/or D-serine in a total concentration from about 1 mM to about 100 mM (more preferably from about 2 mM to about 50 mM, even more preferably from about 3 mM to about 20 mM, most preferably about 5 to 15 mM)

More preferably for the method of the invention, the enzyme capable to metabolize D-serine is selected from the group consisting of
i) the D-serine ammonia-lyase as shown in Table I, and
ii) enzymes having the same enzymatic activity and an identity of at least 80% (preferably at least 85%, more preferably at least 90%, even more preferably at least 95%, most preferably at least 98%) to an amino acid sequence of a D-serine ammonia-lyase as shown in Table I;
iii) enzymes having the same enzymatic activity and an identity of the encoding nucleic acid sequence of at least 80% (preferably at least 85%, more preferably at least 90%, even more preferably at least 95%, most preferably at least 98%) to a nucleic acid sequence of a D-serine ammonia-lyase as shown in Table I, and
iv) enzymes encoded by a nucleic acid sequence capable to hybridize to the complement of the sequence encoding the D-serine ammonia-lyase as shown in Table I,
   and wherein selection is done on a medium comprising D-serine in a concentration from about 1 mM to 100 mM (more preferably from about 5 mM to about 50 mM, even more preferably from about 7 mM to about 30 mM, most preferably about 10 to 20 mM).

More preferably for the method of the invention, the enzyme capable to metabolize D-serine is selected from the group consisting of
i) the E. coli D-serine ammonia-lyase as encoded by SEQ ID NO: 2, and
ii) enzymes having the same enzymatic activity and an identity of at least 80% (preferably at least 85%, more preferably at least 90%, even more preferably at least 95%, most preferably at least 98%) to the amino acid sequence as shown by SEQ ID NO: 2, and
iii) enzymes having the same enzymatic activity and an identity of the encoding nucleic acid sequence of at least 80% (preferably at least 85%, more preferably at least 90%, even more preferably at least 95%, most preferably at least 98%) to the nucleic acid sequence as shown by SEQ ID NO: 1, and
iv) enzymes encoded by a nucleic acid sequence capable to hybridize to the complement of the sequence described by SEQ ID NO: 1,
   and wherein selection is done on a medium comprising D-serine in a concentration from about 1 mM to 100 mM (more preferably from about 5 mM to about 50 mM, even more preferably from about 7 mM to about 30 mM, most preferably about 10 to 20 mM).

“Same activity” in the context of a D-serine ammonia-lyase means the capability to metabolize D-serine, preferably as the most preferred substrate. Metabolization means the lyase reaction specified above. Hybridization under iii) means preferably hybridization under low stringency conditions (with a buffer solution of 30 to 35% formamide, 1 M NaCl, 1% SDS at 37° C., and a wash in 1× to 2×SSC at 50 to 55° C.), more preferably moderate stringency conditions (in 40 to 45% formamide, 1.0 M NaCl, 1% SDS at 37° C., and a wash in 0.5× to 1×SSC at 55 to 60° C.), and most preferably under very stringent conditions (in 50% formamide, 1 M NaCl, 1% SDS at 37° C., and a wash in 0.1×SSC at 60 to 65° C.).

Also more preferably for the method of the invention, the enzyme capable to metabolize D-serine is selected from the group consisting of
i) the D-amino acid oxidase as shown in Table I, and
ii) enzymes having the same enzymatic activity and an identity of at least 80% (preferably at least 85%, more preferably at least 90%, even more preferably at least 95%, most preferably at least 98%) to an amino acid sequence of a D-amino acid oxidase as shown in Table I;
iii) enzymes having the same enzymatic activity and an identity of the encoding nucleic acid sequence of at least 80% (preferably at least 85%, more preferably at least 90%, even more preferably at least 95%, most preferably at least 98%) to a nucleic acid sequence of a D-amino acid oxidase as shown in Table I, and
iv) enzymes encoded by a nucleic acid sequence capable to hybridize to the complement of the sequence encoding the D-amino acid oxidase as shown in Table I,
   and wherein selection is done on a medium comprising D-alanine and/or D-serine in a total concentration from about 1 mM to 100 mM (more preferably from about 2 mM to about 50 mM, even more preferably from about 3 mM to about 20 mM, most preferably about 5 to 15 mM).

Also more preferably for the method of the invention, the enzyme capable to metabolize D-serine and D-alanine is selected from the group consisting of
i) the Rhodotorula gracilis D-amino acid oxidase as encoded by SEQ ID NO: 4, and
ii) enzymes having the same enzymatic activity and an identity of at least 80% (preferably at least 85%, more preferably at least 90%, even more preferably at least 95%, most preferably at least 98%) to the amino acid sequence as shown by SEQ ID NO: 4,
iii) enzymes having the same enzymatic activity and an identity of the encoding nucleic acid sequence of at least 80% (preferably at least 85%, more preferably at least 90%, even more preferably at least 95%, most preferably at least 98%) to the nucleic acid sequence as shown by SEQ ID NO: 3, and
iv) enzymes encoded by a nucleic acid sequence capable to hybridize to the complement of the sequence described by SEQ ID NO: 3,
   and wherein selection is done on a medium comprising D-alanine and/or D-serine in a total concentration from about 1 mM to 100 mM (more preferably from about 2 mM to about 50 mM, even more preferably from about 3 mM to about 20 mM, most preferably about 5 to 15 mM).

Mutants and derivatives of the specified sequences can also comprise enzymes, which are improved in one or more characteristics (Ki, substrate specificity etc.) but still comprise the metabolizing activity regarding D-serine and or D-alanine. Such sequences and proteins also encompass, sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Polynucleotides encoding a candidate enzyme can, for example, be modulated with DNA shuffling protocols. DNA shuffling is a method to rapidly, easily and efficiently introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA encodes an enzyme modified with respect to the enzyme encoded by the template DNA, and preferably has an altered biological activity with respect to the enzyme encoded by the template DNA. DNA shuffling can be based on a process of recursive recombination and mutation, performed by random fragmentation of a pool of related genes, followed by reassembly of the fragments by a polymerase chain reaction-like process. See, e.g., Stemmer 1994 a,b; Crameri 1997; Moore 1997; Zhang 1997; Crameri 1998; U.S. Pat. No. 5,605,793, U.S. Pat. No. 5,837,458, U.S. Pat. No. 5,830,721 and U.S. Pat. No. 5,811,238. The resulting dsdA- or dao-like enzyme encoded by the shuffled DNA may possess different amino acid sequences from the original version of enzyme. Exemplary ranges for sequence identity are specified above.

“Same activity” in the context of a D-amino acid oxidase means the capability to metabolize a broad spectrum of D-amino acids (preferably at least D-serine and/or D-alanine). Metabolization means the oxidase reaction specified above. Hybridization under iii) means preferably hybridization under low stringency conditions (with a buffer solution of 30 to 35% formamide, 1 M NaCl, 1% SDS at 37° C., and a wash in 1× to 2×SSC at 50 to 55° C.), more preferably moderate stringency conditions (in 40 to 45% formamide, 1.0 M NaCl, 1% SDS at 37° C., and a wash in 0.5× to 1×SSC at 55 to 60° C.), and most preferably under very stringent conditions (in 50% formamide, 1 M NaCl, 1% SDS at 37° C., and a wash in 0.1×SSC at 60 to 65° C.).

Preferably, concentrations and times for the selection are specified in detail below. Preferably the selection is done using about 3 to about 15 mM D-alanine or about 7 mM to about 30 mM D-serine. The total selection time under dedifferentiating conditions is preferably from about 3 to 4 weeks.

The D-amino acid metabolizing enzyme of the invention may be expressed in the cytosol, peroxisome, or other intracellular compartment of the plant cell. Compartmentalisation of the D-amino acid metabolizing enzyme may be achieved by fusing the nucleic acid sequence encoding the DAAO polypeptide to a sequence encoding a transit peptide to generate a fusion protein. Gene products expressed without such transit peptides generally accumulate in the cytosol.

In one embodiment, the D-amino acid metabolizing enzyme is functional linked to a promoter, in particular to a promoter which confers—in combination with corresponding further expression regulation signals—expression of the accordingly controlled gene in barley plants. Such a promoter can be for example a constitutive promoter, a promoter which is regulated or a promoter which is active in an suitable tissue or organ.

### 1.1.2 Promoters for Barley Plants

The term “promoter” as used herein is intended to mean a DNA sequence that directs the transcription of a DNA sequence (e.g., a structural gene). Typically, a promoter is located in the 5′ region of a gene, proximal to the transcriptional start site of a structural gene. If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots or meristem.

The term “promoter active in barley plants” means any promoter, whether plant derived or not, which is capable to induce transcription of an operably linked nucleotide sequence in at least one barley cell, tissue, organ or plant at least one time point in development or under dedifferentiated conditions. Such promoter may be a non-plant promoter (e.g., derived from a plant virus or agrobarcterium) or a plant promoter, preferably a monocotyledonous plant promoter.

The person skilled in the art is aware of several promoter which might be suitable for use in barley plants. In this context, expression can be, for example, constitutive, inducible or development-dependent. The following promoters are preferred:

### a) Constitutive Promoters

“Constitutive” promoters refers to those promoters which ensure expression in a large number of, preferably all, tissues over a substantial period of plant development, preferably at all times during plant development. Preferred are: the promoter of the CaMV (cauliflower mosaic virus) 35S transcript (Franck 1980; Odell 1985; Shewmaker 1985; Gardner 1986), the 19S CaMV promoter (U.S. Pat. No. 5,352,605; WO 84/02913; Benfey 1989) are especially preferred, the rice actin promoter (McElroy 1990), the Rubisco small subunit (SSU) promoter (U.S. Pat. No. 4,962,028), the promoter of the nopalin synthase from Agrobacterium, the OCS (octopine synthase) promoter from Agrobacterium, the Smas promoter, the cinnamyl alcohol dehydrogenase promoter (U.S. Pat. No. 5,683,439), the promoters of the vacuolar ATPase subunits, the pEMU promoter (Last 1991); the MAS promoter (Velten 1984) and maize H3 histone promoter (Lepetit 1992; Atanassova 1992), the maize ahas promoter (U.S. Pat. No. 5,750,866) or the ScBV promoter (U.S. Pat. No. 6,489,462).

b) Tissue-Specific or Tissue-Preferred Promoters

Promoters which are furthermore preferred are those which permit a seed-specific expression in monocots such as maize, barley, barley, rye, rice and the like. The promoter of the Ipt2 or Ipt1 gene (WO 95/15389, WO 95/23230) or the promoters described in WO 99/16890 (promoters of the hordein gene, the glutelin gene, the oryzin gene, the prolamin gene, the gliadin gene, the glutelin gene, the zein gene, the casirin gene or the secalin gene) can advantageously be employed. Further preferred are a leaf-specific and light-induced promoter such as that from cab or rubisco (Simpson 1985; Timko 1985); an anther-specific promoter such as that from LAT52 (Twell 1989b); a pollen-specific promoter such as that from Zml3 (Guerrero 1993); and a microspore-preferred promoter such as that from apg (Twell 1993).

Particularly preferred are constitutive promoters. Most preferred are ubiquitin promoters (see below in detail) such as the ubiquitin promoter (Holtorf 1995), and the ubiquitin 1 promoter (Christensen 1989, 1992; Bruce 1989).

### 1.1.2.1 The Ubiquitin Promoter

It one preferred embodiment of the invention the promoter functional in barley plants is an ubiquitin promoter, preferably a ubiquitin promoter derived from a monocotyl plant, e.g. the Zea maize ubiquitin promoter. The use of the ubiquitin promoter results in a consistently high transformation efficiency. The reasons for the superior performance of the ubiquitin promoter are not known. However, it is known that optimal selection needs expression of the selection marker in the relevant cells of the target tissue (which later dedifferentiate and regenerate into the transgenic plants), at the right time and to the right concentration (high enough to ensure efficient selection but not too high to prevent potential negative effects to the cells). The superior function and the effectiveness of maize ubiquitin promoter particularly, may also indicate the need for barley transgenic cells to have sufficient quantity of the D-alanine and/or D-serine metabolizing enzyme (e.g., the DSDA or DAO proteins) that are exogenous (non-native) to barley, in order to survive the selection pressure imposed on them. These effects may be promoter and/or marker dependent, so that certain combinations of promoters and markers outperform others. The ubiquitin promoter thus can be employed as a standard promoter to drive expression of D-amino acid metabolizing enzymes in barley.

Thus, in all preferred embodiment of the invention the D-alanine and/or D-serine metabolizing enzyme is coupled to a ubiquitin promoter, preferably a plant ubiquitin promoter, more preferably a monocotyledonous plant ubiquitin promoter, even more preferably a Zea mays ubiquitin promoter.

The term “ubiquitin promoter” as used herein means the region of genomic DNA up to 5000 base pairs (bp) upstream from either the start codon, or a mapped transcriptional start site, of a ubiquitin, or ubiquitin-like, gene. Ubiquitin is an abundant 76 amino acid polypeptide found in all eukaryotic cells. There are several different genes that encode ubiquitin and their homology at the amino acid level is quite high. For example, human and mouse have many different genes encoding ubiquitin, each located at a different chromosomal locus. Functionally, all ubiquitin genes are critical players in the ubiquitin-dependent proteolytic machinery of the cell. Each ubiquitin gene is associated with a promoter that drives its expression. A ubiquitin promoter is the region of genomic DNA up to 5,000 bp upstream from either the start codon, or a mapped transcriptional start site, of a ubiquitin, or ubiquitin-like, gene.

The term “plant ubiquitin regulatory system” refers to the approximately 2 kb nucleotide sequence 5′ to the translation start site of a plant (preferably the maize) ubiquitin gene and comprises sequences that direct initiation of transcription, regulation of transcription, control of expression level, induction of stress genes and enhancement of expression in response to stress. The regulatory system, comprising both promoter and regulatory functions, is the DNA sequence providing regulatory control or modulation of gene expression.

Various plant ubiquitin genes and their promoters are described (Callis 1989, 1990). Described are promoters from dicotyledonous plants, such as for potato (Garbarino 1992), tobacco (Genschick 1994), tomato (Hoffman 1991), parsely (Kawalleck 1993; WO03/102198, herein incorporated by reference), Arabidopsis (Callis 1990; Holtorf 1995; UBQ8, GenBank Acc.-No: NM_{—}111814; UBQ1, GenBankAcc.-No: NM_{—}115119; UBQ5, GenBankAcc.-No: NM_{—}116090).

Accordingly the ubiquitin promoter of the invention is a DNA fragment (preferably approximately 2 kb in length), said DNA fragment comprising a plant ubiquitin regulatory system, wherein said regulatory system contains a promoter comprising a transcription start site, and—preferably—one or more heat shock elements positioned 5′ to said transcription start site, and—preferably—an intron positioned 3′ to said transcription start site, wherein said regulatory system is capable of regulating expression in maize. Preferably the expression is a constitutive and inducible gene expression such that the level of said constitutive gene expression in monocots is about one-third that obtained in said inducible gene expression in monocots.

Preferred are ubiquitin promoters from monocotyledonous plants. Such promoters are described for maize (Christensen 1992, 1996) Transgenic Res 5:213-218), rice (RUBQ1, RUBQ2, RUBQ3, and RUBQ4; promoters from RUBQ1 and RUBQ2 are suitable for constitutive expression; U.S. Pat. No. 6,528,701).

Most preferred is the ubiquitin promoter from maize as described in U.S. Pat. Nos. 5,614,399, 5,510,474, 6,020,190, 6,054,574, and 6,068,994. The promoter regulates expression of a maize polyubiquitin gene containing 7 tandem repeats. Expression of this maize ubiquitin gene was constitutive at 25° C., and was induced by heat shock at 42° C. The promoter was successfully used in several monocot plants (Christensen 1996). In the maize ubil promoter region, a TATA box was found at position of −30, and two overlapping heat shock sequences, 5′-CTGGTCCCCTCCGA-3′ and CTCGAGATTCCGCT-3′, were found at positions −214 and −204. The canonical CCAAT and the GC boxes were not found in the promoter region, but the sequence 5-CACGGCA-3′ (function unknown) occurred four times, at positions −236, −122, −96, and −91 of the promoter region (Christensen 1992). Promoters and their expression pattern are described for Ubi-1 and Ubi-2 of barley (U.S. Pat. No. 6,054,574; Christensen 1992).

More preferably the ubiquitin promoter is selected from the group consisting of
a) sequences comprising the sequence as described by SEQ ID NO: 5, and
b) sequences comprising at least one fragment of at least 50 (preferably at least 100, more preferably at least 250, even more preferably at least 500, most preferably at least 1000) consecutive base pairs of the sequence as described by SEQ ID NO: 5, and having promoter activity in barley,
c) sequences comprising a sequence having at least 60% (preferably at least 70%, more preferably at least 80%, even more preferably at least 90%, most preferably at least 95%) identity to the sequence as described by SEQ ID NO: 5, and having promoter activity in barley,
d) sequences comprising a sequence hybridizing to the sequence as described by SEQ ID NO: 5, and having promoter activity in barley.

“Promoter activity” in barley means the capability to realized transcription of an operably linked nucleic acid sequence in at least one cell or tissue of a barley plant or derived from a barley plant. Preferably it means a constitutive transcription activity allowing for expression in most tissues and most developmental stages. The heat shock element related activity of the maize ubiquitin promoter may be present but is not required.

Hybridization under d) means preferably hybridization under low stringency conditions (with a buffer solution of 30 to 35% formamide, 1 M NaCl, 1% SDS at 37° C., and a wash in 1× to 2×SSC at 50 to 55° C.), more preferably moderate stringency conditions (in 40 to 45% formamide, 1.0 M NaCl, 1% SDS at 37° C., and a wash in 0.5× to 1×SSC at 55 to 60° C.), and most preferably under very stringent conditions (in 50% formamide, 1 M NaCl, 1% SDS at 37° C., and a wash in 0.1×SSC at 60 to 65° C.).

The sequence described by SEQ ID NO: 5 is the core promoter of the maize ubiquitin promoter. In one preferred embodiment not only the promoter region is employed as a transcription regulating sequence but also a 5′-untranslated region and/or an intron. The ubiquitin promoter is preferably employed in combination with an intron, more preferably with an expression enhancing intron. Such an intron can be the natural intron 1 of the ubil gene (MubG1 contains a 1004-base pair (bp) intron in its 5′ untranslated region; Liu 1995). More preferably the ubiquitin promoter system is characterized by a length of approximately 2 kb, further comprising, in the following order beginning with the 5′ most element and proceeding toward the 3′ terminus of said DNA fragment:
(a) one or more heat shock elements, which elements may or may not be overlapping;
(b) a promoter comprising a transcription start site; and
(c) an intron of about 1 kb in length.

More preferably the region spanning the promoter, the 5′-untranslated region and the first intron of the maize ubiquitin gene are used, even more preferably the region described by SEQ ID NO: 6. Accordingly in another preferred embodiment the ubiquitin promoter utilized in the method of the invention is selected from the group consisting of
a) sequences comprising the sequence as described by SEQ ID NO: 6, and
b) sequences comprising at least one fragment of at least 50 (preferably at least 100, more preferably at least 250, even more preferably at least 500, most preferably at least 1000) consecutive base pairs of the sequence as described by SEQ ID NO: 6, and having promoter activity in barley,
c) sequences comprising a sequence having at least 60% (preferably at least 70%, more preferably at least 80%, even more preferably at least 90%, most preferably at least 95%) identity to the sequence as described by SEQ ID NO: 6, and having promoter activity in barley,
d) sequences comprising a sequence hybridizing to the sequence as described by SEQ ID NO: 6, and having promoter activity in barley.

Hybridization under d) means preferably hybridization under low stringency conditions (with a buffer solution of 30 to 35% formamide, 1 M NaCl, 1% SDS at 37° C., and a wash in 1× to 2×SSC at 50 to 55° C.), more preferably moderate stringency conditions (in 40 to 45% formamide, 1.0 M NaCl, 1% SDS at 37° C., and a wash in 0.5× to 1×SSC at 55 to 60° C.), and most preferably under very stringent conditions (in 50% formamide, 1 M NaCl, 1% SDS at 37° C., and a wash in 0.1×SSC at 60 to 65° C.).

Accordingly the ubiquitin promoter utilized of the invention may also be a fragment of the promoter described by SEQ ID NO: 5 or 6 or a derivative thereof. Fragments may include truncated versions of the promoter as described by SEQ ID NO: 5 or 6, wherein un-essential sequences have been removed. Shortened promoter sequences are of high advantage since they are easier to handle and sometime optimized in their gene expression profile. One efficient, targeted means for preparing shortened or truncated promoters relies upon the identification of putative regulatory elements within the promoter sequence. This can be initiated by comparison with promoter sequences known to be expressed in similar tissue-specific or developmentally unique manner. Sequences, which are shared among promoters with similar expression patterns, are likely candidates for the binding of transcription factors and are thus likely elements that confer expression patterns. Confirmation of these putative regulatory elements can be achieved by deletion analysis of each putative regulatory region followed by functional analysis of each deletion construct by assay of a reporter gene, which is functionally attached to each construct. As such, once a starting promoter sequence is provided, any of a number of different deletion mutants of the starting promoter could be readily prepared.

Functionally equivalent fragments of an ubiquitin promoter (e.g., as described by SEQ ID NO: 5 or 6) can also be obtained by removing or deleting non-essential sequences without deleting the essential one. Narrowing the transcription regulating nucleotide sequence to its essential, transcription mediating elements can be realized in vitro by trial-and-arrow deletion mutations, or in silico using promoter element search routines. Regions essential for promoter activity often demonstrate clusters of certain, known promoter elements. Such analysis can be performed using available computer algorithms such as PLACE (“Plant Cis-acting Regulatory DNA Elements”; Higo 1999), the B10BASE database “Transfac” (Biologische Datenbanken GmbH, Braunschweig; Wingender 2001) or the database PlantCARE (Lescot 2002). Preferably, functional equivalent fragments of one of the transcription regulating nucleotide sequences of the invention comprises at least 100 base pairs, preferably, at least 200 base pairs, more preferably at least 500 base pairs of a transcription regulating nucleotide sequence as described by SEQ ID NO: 5 or 6. More preferably this fragment is starting from the 3′-end of the indicated sequences.

Especially preferred are equivalent fragments of transcription regulating nucleotide sequences, which are obtained by deleting the region encoding the 5′-untranslated region of the mRNA, thus only providing the (untranscribed) promoter region. The 5′-untranslated region can be easily determined by methods known in the art (such as 5′-RACE analysis). Thus, the core promoter region as described by SEQ ID NO: 5 is a fragment of the sequence described by SEQ ID NO: 6, which still comprises the 5′-untranslated region and the intron.

Derivatives may include for example also modified barley promoter sequences, which—for example—do not include two overlapping heat shock elements. Such sequences are for example described in U.S. Pat. Appl. 20030066108 (WO 01/18220).

### 1.1.3 Additional Elements

The expression cassettes (or the vectors in which these are comprised) may comprise further functional elements and genetic control sequences in addition to the promoter active in barley plants (e.g., the ubiquitin promoter). The terms “functional elements” or “genetic control sequences” are to be understood in the broad sense and refer to all those sequences, which have an effect on the materialization or the function of the expression cassette according to the invention. For example, genetic control sequences modify the transcription and translation. Genetic control sequences are described (e.g., Goeddel 1990; Gruber 1993 and the references cited therein).

Preferably, the expression cassettes encompass a promoter active in barley plants (e.g, the ubiquitin promoter) 5′-upstream of the nucleic acid sequence (e.g., encoding the D-amino acid metabolizing enzyme), and 3′-downstream a terminator sequence and polyadenylation signals and, if appropriate, further customary regulatory elements, in each case linked operably to the nucleic acid sequence to be expressed.

Genetic control sequences and functional elements furthermore also encompass the 5′-untranslated regions, introns or non coding 3′-region of genes, such as, for example, the actin-1 intron, or the Adh1-S introns 1, 2 and 6 (general reference: The Maize Handbook, Chapter 116, Freeling and Walbot, Eds., Springer, New York (1994)). It has been demonstrated that they may play a significant role in the regulation of gene expression. Thus, it has been demonstrated that 5′-untranslated sequences can enhance the transient expression of heterologous genes. Examples of translation enhancers which may be mentioned are the tobacco mosaic virus 5′ leader sequence (Gallie 1987) and the like. Furthermore, they may promote tissue specificity (Rouster 1998).

Polyadenylation signals which are suitable as genetic control sequences are plant polyadenylation signals, preferably those which correspond essentially to T-DNA polyadenylation signals from Agrobacterium tumefaciens. Examples of particularly suitable terminator sequences are the OCS (octopine synthase) terminator and the NOS (nopaline synthase) terminator.

Functional elements which may be comprised in a vector include
i) Origins of replication which ensure replication of the expression cassettes or vectors according to the invention in, for example, E. coli. Examples which may be mentioned are ORI (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989),
ii) Multiple cloning sites (MCS) to enable and facilitate the insertion of one or more nucleic acid sequences,
iii) Sequences which make possible homologous recombination, marker deletion, or insertion into the genome of a host organism. Methods based on the cre/lox (Sauer 1998; Odell 1990; Dale 1991), FLP/FRT (Lysnik 1993), or Ac/Ds system (Wader 1987; U.S. Pat. No. 5,225,341; Baker 1987; Lawson 1994) permit a—if appropriate tissue-specific and/or inducible—removal of a specific DNA sequence from the genome of the host organism. Control sequences may in this context mean the specific flanking sequences (e.g., lox sequences), which later allow removal (e.g., by means of cre recombinase) (see also see international patent application PCT/EP 2005/002734),
iv) Elements, for example border sequences, which make possible the Agrobacterium-mediated transfer in plant cells for the transfer and integration into the plant genome, such as, for example, the right or left border of the T-DNA or the vir region.

### 1.2. The Second Expression Cassette

Preferably, the DNA construct inserted into the genome of the target plant comprises at least one second expression cassette, which confers to the barley plant an agronomically relevant trait. This can be achieved by expression of selection markers, trait genes, antisense RNA or double-stranded RNA. The person skilled in the art is aware of numerous sequences which may be utilized in this context, e.g. to increase quality of food and feed, to produce chemicals, fine chemicals or pharmaceuticals (e.g., vitamins, oils, carbohydrates; Dunwell 2000), conferring resistance to herbicides, or conferring male sterility. Furthermore, growth, yield, and resistance against abiotic and biotic stress factors (like e.g., fungi, viruses or insects) may be enhanced. Advantageous properties may be conferred either by overexpressing proteins or by decreasing expression of endogenous proteins by e.g., expressing a corresponding antisense (Sheehy 1988; U.S. Pat. No. 4,801,340; Mol 1990) or double-stranded RNA (Matzke 2000; Fire 1998; Waterhouse 1998; WO 99/32619; WO 99/53050; WO 00/68374; WO 00/44914; WO 00/44895; WO 00/49035; WO 00/63364).

For expression of these sequences all promoters suitable for expression of genes in barley can be employed. Preferably, said second expression construct is not comprising a promoter which is identical to the promoter used to express the D-amino acid metabolizing enzyme. Expression can be, for example, constitutive, inducible or development-dependent. Various promoters are known for expression in monocots like maize, such as the rice actin promoter (McElroy 1990), maize H3 histone promoter (Lepetit 1992; Atanassova 1992), the promoter of a proline-rich protein from barley (WO 91/13991). Promoters which are furthermore preferred are those which permit a seed-specific expression in monocots such as the promoters described in WO 99/16890 (promoters of the hordein gene, the glutelin gene, the oryzin gene, the prolamin gene, the gliadin gene, the glutelin gene, the zein gene, the casirin gene or the secalin gene).

### 2. The Transformation and Selection Method of the Invention

### 2.1 Source and Preparation of the Plant Material

Various plant material can be employed for the transformation procedure disclosed herein. Such plant material may include but is not limited to for example leaf, root, immature and mature embryos, pollen, meristematic tissues, inflorescences but also callus, protoplasts or suspensions of plant cells. Preferably, the plant material is an immature embryo. The material can be pre-treated (e.g., by inducing dedifferentiation prior to transformation) or not pre-treated.

The plant material for transformation (e.g., the immature embryo) can be obtained or isolated from virtually any barley variety or plant. Especially preferred are all barley species especially of the Hordeum family (including winter, spring, two row and six row barley varieties), more especially Hordeum vulgaris subsp. vulgare and Hordeum vulgaris subsp. spontaneum. The method of the invention can be used to produce transgenic plants from spring barley such as for example Golden Promise Hanka, Josefine, as well as from winter barley, such as, for example, Nobila, Siberina. However, it should be pointed out, that the method of the invention is not limited to certain verities but is highly genotype-independent. Barley plants for isolation of immature embryos are grown as known in the art, preferably as described below in the examples

In one preferred embodiment of the invention the method is comprising the following steps
a) isolating an immature embryos of a barley plant, and
b) co-cultivating said isolated immature embryo, which has not been subjected to a dedifferentiation treatment, with a bacterium belonging to genus Rhizobiaceae comprising at least one transgenic T-DNA, said T-DNA comprising
   i) at least one first expression construct comprising a promoter active in said barley plant and operably linked thereto a nucleic acid sequence encoding an enzyme capable to metabolize D-alanine and/or D-serine,
   ii) at least one second expression construct conferring to said barley plant an agronomically valuable trait
c. transferring the co-cultivated immature embryos to a recovering medium, said recovery medium lacking a phytotoxic effective amount of D-serine or D-alanine, and
d. inducing formation of embryogenic callus and selecting transgenic callus on a medium comprising,
   i. an effective amount of at least one auxin compound, and
   ii. D-alanine and/or D-serine in a total concentration from about 1 mM to 100 mM, and
e. regenerating and selecting plants containing the transgenic T-DNA from the said transgenic callus.

In one preferred embodiment the T-DNA further comprises at least one second expression construct conferring to said barley plant an agronomic valuable trait. However also other genes (e.g., reporter genes) can be transformed into the barley plant in combination with the expression cassette for the enzyme capable to metabolize D-alanine and/or D-serine (i.e., the selectable marker).

Thus, in one embodiment, the present invention relates also to a cell culture comprising one or more embryogenic calli derived from immature barley embryo, at least one auxin, preferably in a concentration as described below, and D-alanine and/or D-serine in a total concentration from about 3 mM to 100 mM. In one embodiment, the cell culture also comprises a bacterium belonging to genus Rhizobiaceae.

The term “immature embryo” as used herein means the embryo of an immature seed which is in the stage of early development and maturation after pollination. The developmental stage of the immature embryos to be treated by the method of the present invention are not restricted and the collected embryos may be in any stage after pollination. Preferred embryos are those collected on not less than 2 days after their fertilization. Also preferred are scutella of immature embryos capable of inducing dedifferentiated calli having an ability to regenerate normal plants after having been transformed by the method mentioned below.

In a preferred embodiment the immature embryo is one in the stage of not less than 10 days after pollination. More preferably, immature embryos are isolated from spikes 12 to 14 days after pollination (DAP). Exact timing of harvest varies depending on growth conditions and barley variety. The size of immature embryos is a good indication of their stage of development. The optimal length of immature embryos for transformation is about 1 to 1.2 mm, including the length of the scutellum. The embryo should be translucent, not opaque.

In a preferred embodiment of the invention, the immature embryos bisected longitudinally through the root and shoot meristems are isolated and directly placed on the surface of a solidified co-cultivation medium. Just before infection the explants are arranged with a scutellum side up. With the present invention, the Agrobacterium infection step takes place on the co-cultivation medium after dripping bacterial suspension on the explants surface.

Preferably, the immature embryo is subjected to transformation (co-cultivation) without dedifferentiating pretreatment. Treatment of the immature embryos with a cell wall degrading enzyme or injuring (e.g., cutting with scalpels or perforation with needles) is optional. However, this degradation or injury step is not necessary and is omitted in a preferred embodiment of the invention.

The term “dedifferentiation”, “dedifferentiation treatment” or “dedifferentiation pretreatment” means a process of obtaining cell clusters, such as callus, that show unorganized growth by culturing differentiated cells of plant tissues on a dedifferentiation medium. More specifically, the term “dedifferentiation” as used herein is intended to mean the process of formation of rapidly dividing cells without particular function in the scope of the plant body. These cells often possess an increased potency with regard to its ability to develop into various plant tissues. Preferably the term is intended to mean the reversion of a differentiated or specialized tissues to a more pluripotent or totipotent (e.g., embryonic) form. Dedifferentiation may lead to reprogramming of a plant tissue (revert first to undifferentiated, non-specialized cells. then to new and different paths). The term “totipotency” as used herein is intended to mean a plant cell containing all the genetic and/or cellular information required to form an entire plant. Dedifferentiation can be initiated by certain plant growth regulators (e.g., auxin and/or cytokinin compounds), especially by certain combinations and/or concentrations thereof.

### 2.2 Transformation Procedures

### 2.2.1 General Techniques

A DNA construct may according to the invention advantageously be introduced into cells using vectors into which said DNA construct is inserted. Examples of vectors may be plasmids, cosmids, phages, viruses, retroviruses or Agrobacteria. In an advantageous embodiment, the expression cassette is introduced by means of plasmid vectors. Preferred vectors are those, which enable the stable integration of the expression cassette into the host genome.

The DNA construct can be introduced into the target plant cells and/or organisms by any of the several means known to those of skill in the art, a procedure which is termed transformation (see also Keown 1990). Various transformation procedures suitable for barley have been described.

For example, the DNA constructs can be introduced directly to plant cells using ballistic methods, such as DNA particle bombardment, or the DNA construct can be introduced using techniques such as electroporation and microinjection of a cell. Particle-mediated transformation techniques (also known as “biolistics”) are described in, e.g., EP-A1 270,356; U.S. Pat. No. 5,100,792, EP-A-444 882, EP-A-434 616; Klein 1987; Vasil 1993; and Becker 1994). These methods involve penetration of cells by small particles with the nucleic acid either within the matrix of small beads or particles, or on the surface. The biolistic PDS-1000 Gene Gun (Biorad, Hercules, Calif.) uses helium pressure to accelerate DNA-coated gold or tungsten microcarriers toward target cells. The process is applicable to a wide range of tissues and cells from organisms, including plants. Other transformation methods are also known to those of skill in the art.

Other techniques include microinjection (WO 92/09696, WO 94/00583, EP-A 331 083, EP-A 175 966, Green 1987), polyethylene glycol (PEG) mediated transformation (Paszkowski 1984; Lazzeri 1995), liposome-based gene delivery (WO 93/24640; Freeman 1984), electroporation (EP-A 290 395, WO 87/06614; Fromm 1985; Shimamoto 1992).

In the case of injection or electroporation of DNA into plant cells, the DNA construct to be transformed not need to meet any particular requirement (in fact the “naked” expression cassettes can be utilized). Simple plasmids such as those of the pUC series may be used.

In addition and preferred to these “direct” transformation techniques, transformation can also be carried out by bacterial infection by means of soil born bacteria such as Agrobacterium tumefaciens or Agrobacterium rhizogenes. These strains contain a plasmid (Ti or Ri plasmid). Part of this plasmid, termed T-DNA (transferred DNA), is transferred to the plant following Agrobacterium infection and integrated into the genome of the plant cell. Although originally developed for dicotyledonous plants, Agrobacterium mediated transformation is employed for transformation methods of monocots (Hiei 1994). Transformation is described e.g., for rice, maize, barley, oat, and barley (reviewed in Shimamoto 1994; Vasil et al. 1992; Vain 1995; Vasil 1996; Wan & Lemaux 1994).

For Agrobacterium-mediated transformation of plants, the DNA construct of the invention may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the cell is infected by the bacteria. Thus, the DNA construct of the invention is preferably integrated into specific plasmids suitable for Agrobacterium mediated transformation, either into a shuttle, or intermediate, vector or into a binary vector). If, for example, a Ti or Ri plasmid is to be used for the transformation, at least the right border, but in most cases the right and the left border, of the Ti or Ri plasmid T-DNA is linked with the expression cassette to be introduced as a flanking region. Binary vectors, capable of replication both in E. coli and in Agrobacterium, are preferably used. They can be transformed directly into Agrobacterium (Holsters 1978).

### 2.2.2 Agrobacterium Mediated Transformation (Co-Cultivation)

The soil-borne bacterium employed for transfer of an T-DNA into the immature embryo can be any specie of the Rhizobiaceae family. The Rhizobiaceae family comprises the genera Agrobacterium, Rhizobium, Sinorhizobium, and Allorhizobium are genera within the bacterial family and has been included in the alpha-2 subclass of Proteobacteria on the basis of ribosomal characteristics. Members of this family are aerobic, Gram-negative. The cells are normally rod-shaped (0.6-1.0 μm by 1.5-3.0 μm), occur singly or in pairs, without endospore, and are motile by one to six peritrichous flagella. Considerable extracellular polysaccharide slime is usually produced during growth on carbohydrate-containing media. Especially preferred are Rhizobiaceae such as Sinorhizobium meliloti, Sinorhizobium medicae, Sinorhizobium fredii, Rhizobium sp. NGR234, Rhizobium sp. BR816, Rhizobium sp. N33, Rhizobium sp. GRH2, Sinorhizobium saheli, Sinorhizobium terangae, Rhizobium leguminosarum biovar trifolii, Rhizobium leguminosarum biovar viciae, Rhizobium leguminosarum biovar phaseoli, Rhizobium tropici, Rhizobium etli, Rhizobium galegae, Rhizobium gallicum, Rhizobium giardinii, Rhizobium hainanense, Rhizobium mongolense, Rhizobium lupini, Mesorhizobium loti, Mesorhizobium huakuii, Mesorhizobium ciceri, Mesorhizobium mediterraneium, Mesorhizobium tianshanense, Bradyrhizobium elkanni, Bradyrhizobium japonicum, Bradyrhizobium liaoningense, Azorhizobium caulinodans, Allobacterium undicola, Phyllobacterium myrsinacearum, Agrobacterium tumefaciens, Agrobacterium radiobacter, Agrobacterium rhizogenes, Agrobacterium vitis, and Agrobacterium rubi. Preferred are also the strains and method described in Broothaerts W et al. (2005) Nature 433:629-633.

The monophyletic nature of Agrobacterium, Allorhizobium and Rhizobium and their common phenotypic generic circumscription support their amalgamation into a single genus, Rhizobium. The classification and characterization of Agrobacterium strains including differentiation of Agrobacterium tumefaciens and Agrobacterium rhizogenes and their various opine-type classes is a practice well known in the art (see for example Laboratory guide for identification of plant pathogenic bacteria, 3rd edition. (2001) Schaad, Jones, and Chun (eds.) ISBN 0890542635; for example the article of Moore et al. published therein). Recent analyses demonstrate that classification by its plant-pathogenic properties may not be justified. Accordingly more advanced methods based on genome analysis and comparison (such as 16S rRNA sequencing; RFLP, Rep-PCR, etc.) are employed to elucidate the relationship of the various strains (see for example Young 2003, Farrand 2003, de Bruijn 1996, Vinuesa 1998). The phylogenetic relationships of members of the genus Agrobacterium by two methods demonstrating the relationship of Agrobacterium strains K599 are presented in Llob 2003.

It is known in the art that not only Agrobacterium but also other soil-borne bacteria are capable to mediate T-DNA transfer provided that they the relevant functional elements for the T-DNA transfer of an Ti- or Ri-plasmid (Klein & Klein 1953; Hooykaas 1977; van Veen 1988).

Preferably, the soil-born bacterium is of the genus Agrobacterium. The term “Agrobacterium” as used herein refers to a soil-borne, Gram-negative, rod-shaped phytopathogenic bacterium. The species of Agrobacterium, Agrobacterium tumefaciens (syn. Agrobacterium radiobacter), Agrobacterium rhizogenes, Agrobacterium rubi and Agrobacterium vitis, together with Allorhizobium undicola, form a monophyletic group with all Rhizobium species, based on comparative 16S rDNA analyses (Sawada 1993, Young 2003). Agrobacterium is an artificial genus comprising plant-pathogenic species.

The term Ti-plasmid as used herein is referring to a plasmid, which is replicable in Agrobacterium and is in its natural, “armed” form mediating crown gall in Agrobacterium infected plants. Infection of a plant cell with a natural, “armed” form of a Ti-plasmid of Agrobacterium generally results in the production of opines (e.g., nopaline, agropine, octopine etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria. A disarmed Ti-plasmid is understood as a Ti-plasmid lacking its crown gall mediating properties but otherwise providing the functions for plant infection. Preferably, the T-DNA region of said “disarmed” plasmid was modified in a way, that beside the border sequences no functional internal Ti-sequences can be transferred into the plant genome. In a preferred embodiment—when used with a binary vector system—the entire T-DNA region (including the T-DNA borders) is deleted.

The term Ri-plasmid as used herein is referring to a plasmid which is replicable in Agrobacterium and is in its natural, “armed” form mediating hairy-root disease in Agrobacterium infected plants. Infection of a plant cell with a natural, “armed” form of an Ri-plasmid of Agrobacterium generally results in the production of opines (specific amino sugar derivatives produced in transformed plant cells such as e.g., agropine, cucumopine, octopine, mikimopine etc.) by the infected cell. Agrobacterium rhizogenes strains are traditionally distinguished into subclasses in the same way A. tumefaciens strains are. The most common strains are agropine-type strains (e.g., characterized by the Ri-plasmid pRi-A4), mannopine-type strains (e.g., characterized by the Ri-plasmid pRi8196) and cucumopine-type strains (e.g., characterized by the Ri-plasmid pRi2659). Some other strains are of the mikimopine-type (e.g., characterized by the Ri-plasmid pRi1723). Mikimopine and cucumopine are stereo isomers but no homology was found between the pRi plasmids on the nucleotide level (Suzuki 2001). A disarmed Ri-plasmid is understood as a Ri-plasmid lacking its hairy-root disease mediating properties but otherwise providing the functions for plant infection. Preferably, the T-DNA region of said “disarmed” Ri plasmid was modified in a way, that beside the border sequences no functional internal Ri-sequences can be transferred into the plant genome. In a preferred embodiment—when used with a binary vector system—the entire T-DNA region (including the T-DNA borders) is deleted.

The Ti and Ri plasmids of A. tumefaciens and A. rhizogenes, respectively, carry genes responsible for genetic transformation of the plant (Kado 1991). Vectors are based on the Agrobacterium Ti- or Ri-plasmid and utilize a natural system of DNA transfer into the plant genome. As part of this highly developed parasitism Agrobacterium transfers a defined part of its genomic information (the T-DNA; flanked by about 25 bp repeats, named left and right border) into the chromosomal DNA of the plant cell (Zupan 2000). By combined action of the so called vir genes (part of the original Ti-plasmids) said DNA-transfer is mediated. For utilization of this natural system, Ti-plasmids were developed which lack the original tumor inducing genes (“disarmed vectors”). In a further improvement, the so called “binary vector systems”, the T-DNA was physically separated from the other functional elements of the Ti-plasmid (e.g., the vir genes), by being incorporated into a shuttle vector, which allowed easier handling (EP-A 120 516; U.S. Pat. No. 4,940,838). These binary vectors comprise (beside the disarmed T-DNA with its border sequences), prokaryotic sequences for replication both in Agrobacterium and E. coli. It is an advantage of Agrobacterium-mediated transformation that in general only the DNA flanked by the borders is transferred into the genome and that preferentially only one copy is inserted. Descriptions of Agrobacterium vector systems and methods for Agrobacterium-mediated gene transfer are known in the art (Miki 1993; Gruber 1993; Moloney 1989).

Hence, for Agrobacteria-mediated transformation the genetic composition (e.g., comprising an expression cassette) is integrated into specific plasmids, either into a shuttle or intermediate vector, or into a binary vector. If a Ti or Ri plasmid is to be used for the transformation, at least the right border, but in most cases the right and left border, of the Ti or Ri plasmid T-DNA is linked to the expression cassette to be introduced in the form of a flanking region. Binary vectors are preferably used. Binary vectors are capable of replication both in E. coli and in Agrobacterium. They may comprise a selection marker gene and a linker or polylinker (for insertion of e.g. the expression cassette to be transferred) flanked by the right and left T-DNA border sequence. They can be transferred directly into Agrobacterium (Holsters 1978). The selection marker gene permits the selection of transformed Agrobacteria and is, for example, the nptII gene, which confers resistance to kanamycin. The Agrobacterium which acts as the host organism in this case should already contain a plasmid with the vir region. The latter is required for transferring the T-DNA to the plant cell. An Agrobacterium transformed in this way can be used for transforming plant cells. The use of T-DNA for transforming plant cells has been studied and described intensively (EP 120 516; Hoekema 1985; An 1985).

Common binary vectors are based on “broad host range”-plasmids like pRK252 (Bevan 1984) or pTJS75 (Watson 1985) derived from the P-type plasmid RK2. Most of these vectors are derivatives of pBIN19 (Bevan 1984). Various binary vectors are known, some of which are commercially available such as, for example, pBI101.2 or pBIN19 (Clontech Laboratories, Inc. USA). Additional vectors were improved with regard to size and handling (e.g. pPZP; Hajdukiewicz 1994). Improved vector systems are described also in WO 02/00900.

Preferably the soil-borne bacterium is a bacterium belonging to family Agrobacterium, more preferably a disarmed Agrobacterium tumefaciens or rhizogenes strain. In a preferred embodiment, Agrobacterium strains for use in the practice of the invention include octopine strains, e.g., LBA4404 or agropine strains, e.g., EHA101-[pEHA101] or EHA105-[pEHA105]. Suitable strains of A. tumefaciens for DNA transfer are for example EHA101pEHA101 (Hood 1986), EHA105-[pEHA105] (Li 1992), LBA4404-[pAL4404] (Hoekema 1983), C58C1-[pMP90] (Koncz & Schell 1986), and C58C1-[pGV2260] (Deblaere 1985). Other suitable strains are Agrobacterium tumefaciens C58, a nopaline strain. Other suitable strains are A. tumefaciens C58C1 (Van Larebeke 1974), A136 (Watson 1975) or LBA4011 (Klapwijk 1980). In another preferred embodiment the soil-borne bacterium is a disarmed strain variant of Agrobacterium rhizogenes strain K599 (NCPPB 2659). Such strains are described in U.S. provisional application No. 60/606,789, filed Sep. 2, 2004, hereby incorporated entirely by reference.

Preferably, these strains are comprising a disarmed plasmid variant of a Ti- or Ri-plasmid providing the functions required for T-DNA transfer into plant cells (e.g., the vir genes). In a preferred embodiment, the Agrobacterium strain used to transform the plant tissue pre-cultured with the plant phenolic compound contains a L,L-succinamopine type Ti-plasmid, preferably disarmed, such as pEHA101. In another preferred embodiment, the Agrobacterium strain used to transform the plant tissue pre-cultured with the plant phenolic compound contains an octopine-type Ti-plasmid, preferably disarmed, such as pAL4404. Generally, when using octopine-type Ti-plasmids or helper plasmids, it is preferred that the virF gene be deleted or inactivated (Jarschow 1991).

The method of the invention can also be used in combination with particular Agrobacterium strains, to further increase the transformation efficiency, such as Agrobacterium strains wherein the vir gene expression and/or induction thereof is altered due to the presence of mutant or chimeric virA or virG genes (e.g. Hansen 1994; Chen and Winans 1991; Scheeren-Groot 1994). Preferred are further combinations of Agrobacterium tumefaciens strain LBA4404 (Hiei 1994) with super-virulent plasmids. These are preferably pTOK246-based vectors (Ishida 1996).

A binary vector or any other vector can be modified by common DNA recombination techniques, multiplied in E. coli, and introduced into Agrobacterium by e.g., electroporation or other transformation techniques (Mozo 1991).

Agrobacterium is preferably grown and used in a manner similar to that described in Ishida (Ishida 1996). The vector comprising Agrobacterium strain may, for example, be grown for 3 days on YP medium (5 g/l yeast extract, 10 g/l peptone, 5 g/l NaCl, 15 g/l agar, pH 6.8) supplemented with the appropriate antibiotic (e.g., 50 mg/l spectinomycin). Bacteria are collected with a loop from the solid medium and resuspended. In a preferred embodiment of the invention, Agrobacterium cultures are started by use of aliquots frozen at −80° C.

The transformation of the immature embryos by the Agrobacterium may be carried out by merely contacting the immature embryos with the Agrobacterium. The concentration of Agrobacterium used for infection and co-cultivation may need to be varied. For example, a cell suspension of the Agrobacterium having a population density of approximately from 10⁵ to 10¹¹, preferably 10⁶ to 10¹⁰, more preferably about 10⁸ cells or cfu/ml is prepared and the immature embryos are immersed in this suspension for about 3 minutes to 5 hours, preferably for about 1 hour at 26° C. The resulting immature embryos are then cultured on a solid medium for several days together with the Agrobacterium (co-cultivation).

In another preferred embodiment for the infection and co-cultivation step a suspension of the soil-borne bacterium (e.g., Agrobacteria) in the co-cultivation or infection medium is directly applied to each embryo, and excess amount of liquid covering the embryo is removed. Removal can be done by various means, preferably through either air-drying or absorbing. This is saving labor and time and is reducing unintended Agrobacterium-mediated damage by excess Agrobacterium usage. In a preferred embodiment from about 1 to about 10 μl of a suspension of the soil-borne bacterium (e.g., Agrobacteria) are employed. Preferably, the immature embryo is infected with Agrobacterium directly on the co-cultivation medium. Preferably, the bacterium is employed in concentration of 10⁶ to 10¹¹ cfu/ml.

For Agrobacterium treatment of isolated immature embryos, the bacteria are resuspended in a plant compatible co-cultivation medium. Supplementation of the co-culture medium with ethylene inhibitors (e.g., silver nitrate), phenol-absorbing compounds (like polyvinylpyrrolidone, Perl 1996) or antioxidants (such as thiol compounds, e.g., dithiothreitol, L-cysteine, Olhoft 2001) which can decrease tissue necrosis due to plant defense responses (like phenolic oxidation) may further improve the efficiency of Agrobacterium-mediated transformation. In another preferred embodiment, the co-cultivation medium of comprises least one thiol compound, preferably selected from the group consisting of sodium thiolsulfate, dithiotrietol (DTT) and cysteine. Preferably the concentration is between about 1 mM and 10 mM of L-Cysteine, 0.1 mM to 5 mM DTT, and/or 0.1 mM to 5 mM sodium thiolsulfate. Preferably, the medium employed during co-cultivation comprises from about 1 μM to about 10 μM of silver nitrate and/or (preferably “and”) from about 50 mg/L to about 1,000 mg/L of L-Cysteine. This results in a highly reduced vulnerability of the immature embryo against Agrobacterium-mediated damage (such as induced necrosis) and highly improves overall transformation efficiency.

A range of co-cultivation periods from a few hours to 10 days may be employed. The co-cultivation of Agrobacterium with the isolated immature embryos is in general carried out for about 12 hours to about 7 days, preferably about 4 days to about 6 days at about 24° C. to about 26° C. (more preferably in medium PAW-1 or PAB-1 as described below in the Examples).

In an improved embodiment of the invention the isolated immature embryos and/or the Agrobacteria may be treated with a phenolic compound prior to or during the Agrobacterium co-cultivation. “Plant phenolic compounds” or “plant phenolics” suitable within the scope of the invention are those isolated substituted phenolic molecules which are capable to induce a positive chemotactic response, particularly those who are capable to induce increased vir gene expression in a Ti-plasmid containing Agrobacterium sp., particularly a Ti-plasmid containing Agrobacterium tumefaciens. Methods to measure chemotactic responses towards plant phenolic compounds have been like e.g., described (Ashby 1988) and methods to measure induction of vir gene expression are also well known (Stachel 1985; Bolton 1986). The pre-treatment and/or treatment during Agrobacterium co-cultivation has at least two beneficial effects: Induction of the vir genes of Ti plasmids or helper plasmids (Van Wordragen 1992; Jacq 1993; James 1993; Guivarc'h 1993), and enhancement of the competence for incorporation of foreign DNA into the genome of the plant cell.

Accordingly, in one embodiment, the present invention relates also to a cell culture comprising one or more embryogenic calli derived from immature barley embryo, at least one auxin, preferably in a concentration as described below, D-alanine and/or D-serine in a total concentration from about 1 mM to about 100 mM and at least one plant phenolic compound, e.g. one or more plant phenolic compounds listed below. In one embodiment, the cell culture also comprises a bacterium belonging to genus Rhizobiaceae.

Preferred plant phenolic compounds are those found in wound exudates of plant cells. One of the best known plant phenolic compounds is acetosyringone, which is present in a number of wounded and intact cells of various plants, albeit in different concentrations. However, acetosyringone (3,5-dimethoxy-4-hydroxyacetophenone) is not the only plant phenolic which can induce the expression of vir genes. Other examples are [symbol] hydroxy-acetosyringone, sinapinic acid (3,5-dimethoxy-4-hydroxycinnamic acid), syringic acid (4-hydroxy-3,5 dimethoxybenzoic acid), ferulic acid (4-hydroxy-3-methoxycinnamic acid), catechol (1,2-dihydroxybenzene), p-hydroxybenzoic acid (4-hydroxybenzoic acid), [symbol] -resorcylic acid (2,4-dihydroxybenzoic acid), protocatechuic acid (3,4-dihydroxybenzoic acid), pyrogallic acid (2,3,4-trihydroxybenzoic acid), gallic acid (3,4,5-trihydroxybenzoic acid) and vanillin (3-methoxy-4-hydroxybenzaldehyde), and these phenolic compounds are known or expected to be able to replace acetosyringone in the cultivation media with similar results. As used herein, the mentioned molecules are referred to as plant phenolic compounds.

Plant phenolic compounds can be added to the plant culture medium either alone or in combination with other plant phenolic compounds. A particularly preferred combination of plant phenolic compounds comprises at least acetosyringone and p-hydroxybenzoic acid, but it is expected that other combinations of two, or more, plant phenolic compounds will also act synergistically in enhancing the transformation efficiency.

Moreover, certain compounds, such as osmoprotectants (e.g. L-proline preferably at a concentration of about 200-1000 mg/L or betaine), phytohormes (inter alia NAA), opines, or sugars, act synergistically when added in combination with plant phenolic compounds.

In one embodiment of the invention, it is preferred that the plant phenolic compound, particularly acetosyringone is added to the medium prior to contacting the isolated immature embryos with Agrobacteria for 1 to 24 h. The exact period, in which the cultured cells are incubated in the medium containing the plant phenolic compound such as acetosyringone, is believed not to be critical and only limited by the time the immature embryos start to differentiate.

The concentration of the plant phenolic compound in the medium is also believed to have an effect on the development of competence for integrative transformation. The optimal concentration range of plant phenolic compounds in the medium may vary depending on the barley variety from which the immature embryos derived, but it is expected that about 100 μM to 700 μM is a suitable concentration for many purposes. However, concentrations as low as approximately 25 μM can be used to obtain a good effect on transformation efficiency. Likewise, it is expected that higher concentrations up to approximately 1000 μM will yield similar effects. Comparable concentrations apply to other plant phenolic compounds, and optimal concentrations can be established easily by experimentation in accordance with this invention.

Agrobacteria to be co-cultivated with the isolated immature embryos can be either pre-incubated with acetosyringone or another plant phenolic compound, as known by the person skilled in the art, or used directly after isolation from their culture medium. Particularly suited induction conditions for Agrobacterium tumefaciens have been described by Vernade et al. (1988). Efficiency of transformation with Agrobacterium can be enhanced by numerous other methods known in the art like for example vacuum infiltration (WO 00/58484), heat shock and/or centrifugation, addition of silver nitrate, sonication etc.

It has been observed within this invention that transformation efficacy of the isolated immature embryos by Agrobacterium can be significantly improved by keeping the pH of the co-cultivation medium in a range from 5.4 to 6.4, preferably 5.6 to 6.2, especially preferably 5.8 to 6.0. In an improved embodiment of the invention stabilization of the pH in this range is mediated by a combination of MES and potassium hydrogenphosphate buffers.

### 2.3 Recovery

Transformed cells, i.e. those which comprise the DNA integrated into the DNA of the host cell, can be selected from untransformed cells preferably using the selection method of the invention.

Prior to a transfer to a recovery and/or selection medium, especially in case of Agrobacterium-mediated transformation, certain other intermediate steps may be employed. For example, any Agrobacteria remaining from the co-cultivation step may be removed (e.g., by a washing step). To prevent re-growth of said bacteria, the subsequently employed recovery and/or selection medium preferably comprises a bacteriocide (antibiotic) suitable to prevent Agrobacterium growth. Preferred bactericidal antibiotics to be employed are e.g., cefotaxime 500 mg/l or 160 mg/l mg/L Timentin™ (GlaxoSmithKline; a mixture of ticarcillin disodium and clavulanate potassium; 0.8 g Timentin™ contains 50 mg clavulanic acid with 750 mg ticarcillin. Chemically, ticarcillin disodium is N-(2-Carboxy-3,3-dimethyl-7-oxo-4-thia-1-azabicyclo[3.2.0]hept-6-yl)-3-thio-phenemalonamic acid disodium salt. Chemically, clavulanate potassium is potassium (Z)-(2R,5R)-3-(2-hydroxyethylidene)-7-oxo-4-oxa-1-azabicyclo[3.2.0]heptane-2-carboxylate).

It is preferred that the step directly following the transformation procedure (e.g., co-cultivation) is not comprising an effective, phytotoxic amount of D-alanine and/or D-serine or derivatives thereof (which are subsequently used for transformation). Thus, this step is intended to allow for regeneration of the transformed tissue, to promote initiation of embryogenic callus formation in the Agrobacterium-infected embryo, and kill the remaining Agrobacterium cells. Accordingly, in a preferred embodiment the method of the invention comprises the step of transferring the transformed target tissue (e.g., the co-cultivated immature embryos) to a recovering medium (used in step c) comprising
i. an effective amount of at least one antibiotic that inhibits or suppresses the growth of the soil-borne bacteria, and/or (preferably “and”)
ii. L-proline in a concentration from about 0.5 g/l to about 2 g/l, and/or (preferably “and”)

Thus, in one embodiment, the present invention relates, to a recovery medium comprising an effective amount of at least one antibiotic that inhibits or suppresses the growth of the soil-borne bacteria, and/or (preferably “and”) L-proline in a concentration from about 0.5 g/l to about 2 g/l. Preferably, the medium comprises further the transformed target tissue (e.g., the co-cultivated immature embryos).

Preferably said recovery medium does not comprise an effective, phytotoxic amount of D-alanine and/or D-serine or a derivative thereof. The recovery medium may further comprise an effective amount of at least one plant growth regulator (e.g., an effective amount of at least one auxin compound). Thus the recovery medium of step c) preferably comprises
i. an effective amount of at least one antibiotic that inhibits or suppresses the growth of the soil-borne bacteria, and
ii. L-proline in a concentration from about 0.5 g/l to about 2 g/l, and
iv. an effective amount of at least one auxin compound.

Examples for preferred recovery media are given below in the Examples (2 and 3). The recovery period (i.e. the period under dedifferentiating conditions without a selection pressure by a phytotoxic amount of D-alanine and/or D-seine) may last for about 1 day to about 30 days, preferably about 5 days to about 20 days, more preferably about 7 days. in the dark A medium such as PAW-2 or PAB-2 (see Examples) can be employed for this purpose. Preferably, the scutellum side is kept up during this time and do not embedded into the media.

### 2.4 Selection

After the recovery step the target tissue (e.g., the immature embryos) are transferred to and incubated on a selection medium. The selection medium comprises D-alanine and/or D-serine or a derivative thereof in a phytotoxic concentration (i.e., in a concentration which either terminates or at least retard the growth of the non-transformed cells). The term “phytotoxic”, “phytotoxicity” or “phytotoxic effect” as used herein is intended to mean any measurable, negative effect on the physiology of a plant or plant cell resulting in symptoms including (but not limited to) for example reduced or impaired growth, reduced or impaired photosynthesis, reduced or impaired cell division, reduced or impaired regeneration (e.g., of a mature plant from a cell culture, callus, or shoot etc.), reduced or impaired fertility etc. Phytotoxicity may further include effects like e.g., necrosis or apoptosis. In a preferred embodiment results in an reduction of growth or regenerability of at least 50%, preferably at least 80%, more preferably at least 90% in comparison with a plant which was not treated with said phytotoxic compound.

Thus, in one embodiment, the present invention relates to an selection medium comprising the target tissue (e.g., embryonic wheat calli, i.e. the transformed and regenerated barley immature embryos described above) and D-alanine and/or D-serine or a derivative thereof in a phytotoxic concentration as described below.

The specific compound employed for selection is chosen depending on which marker protein is expressed. For example in cases where the E. coli D-serine ammonia-lyase is employed, selection is done on a medium comprising D-serine. In cases where the Rhodotorula gracilis D-amino acid oxidase is employed, selection is done on a medium comprising D-alanine and/or D-serine.

The fact that D-amino acids are employed does not rule out the presence of L-amino acid structures or L-amino acids. For some applications it may be preferred (e.g., for cost reasons) to apply a racemic mixture of D- and L-amino acids (or a mixture with enriched content of D-amino acids). Preferably, the ratio of the D-amino acid to the corresponding L-enantiomer is at least 1:1, preferably 2:1, more preferably 5:1, most preferably 10:1 or 100:1. The use of D-alanine has the advantage that racemic mixtures of D- and L-alanine can be applied without disturbing or detrimental effects of the L-enantiomer. Therefore, in an improved embodiment a racemic mixture of D/L-alanine is employed as compound

The term “derivative” with respect to D-alanine or D-serine means chemical compound which are comprising the respective D-amino acid structure of D-alanine or D-serine, but are chemically modified. As used herein the term a “D-amino acid structure” (such as a “D-serine structure”) is intended to include the D-amino acid, as well as analogues, derivatives and mimetics of the D-amino acid that maintain the functional activity of the compound. As used herein, a “derivative” also refers to a form of D-serine or D-alanine in which one or more reaction groups on the compound have been derivatized with a substituent group. The D-amino acid employed may be modified by an amino-terminal or a carboxy-terminal modifying group or by modification of the side-chain. The amino-terminal modifying group may be—for example—selected from the group consisting of phenylacetyl, diphenylacetyl, triphenylacetyl, butanoyl, isobutanoyl hexanoyl, propionyl, 3-hydroxybutanoyl, 4-hydroxybutanoyl, 3-hydroxypropionoyl, 2,4-dihydroxybutyroyl, 1-Adamantanecarbonyl, 4-methylvaleryl, 2-hydroxyphenylacetyl, 3-hydroxyphenylacetyl, 4-hydroxyphenylacetyl, 3,5-dihydroxy-2-naphthoyl, 3,7-dihydroxy-2-napthoyl, 2-hydroxycinnamoyl, 3-hydroxycinnamoyl, 4-hydroxycinnamoyl, hydrocinnamoyl, 4-formylcinnamoyl, 3-hydroxy-4-methoxycinnamoyl, 4-hydroxy-3-methoxycinnamoyl, 2-carboxycinnamoyl, 3,4-dihydroxyhydrocinnamoyl, 3,4-dihydroxycinnamoyl, trans-Cinnamoyl, (±)-mandelyl, (±)-mandelyl-(±)-mandelyl, glycolyl, 3-formylbenzoyl, 4-formylbenzoyl, 2-formylphenoxyacetyl, 8-formyl-1-napthoyl, 4-(hydroxymethyl)benzoyl, 3-hydroxybenzoyl, 4-hydroxybenzoyl, 5-hydantoinacetyl, L-hydroorotyl, 2,4-dihydroxybenzoyl, 3-benzoylpropanoyl, (±)-2,4-dihydroxy-3,3-dimethyl butanoyl, DL-3-(4-hydroxyphenyl)lactyl, 3-(2-hydroxyphenyl)propionyl, 4-(2-hydroxyphenyl)propionyl, D-3-phenyllactyl, 3-(4-hydroxyphenyl)propionyl, L-3-phenyllactyl, 3-pyridylacetyl, 4-pyridylacetyl, isonicotinoyl, 4-quinolinecarboxyl, 1-isoquinolinecarboxyl and 3-isoquinolinecarboxyl. The carboxy-terminal modifying group may be—for example—selected from the group consisting of an amide group, an alkyl amide group, an aryl amide group and a hydroxy group. The “derivative” as used herein are intended to include molecules which mimic the chemical structure of a respective D-amino acid structure and retain the functional properties of the D-amino acid structure. Approaches to designing amino acid or peptide analogs, derivatives and mimetics are known in the art (e.g., see Farmer 1980; Ball 1990; Morgan 1989; Freidinger 1989; Sawyer 1995; Smith 1995; Smith 1994; Hirschman 1993). Other possible modifications include N-alkyl (or aryl) substitutions, or backbone crosslinking to construct lactams and other cyclic structures. Other derivatives include C-terminal hydroxymethyl derivatives, O-modified derivatives (e.g., C-terminal hydroxymethyl benzyl ether), N-terminally modified derivatives including substituted amides such as alkylamides and hydrazides. Furthermore, D-amino acid structure comprising herbicidal compounds may be employed. Such compounds are for example described in U.S. Pat. No. 5,059,239, and may include (but shall not be limited to) N-benzoyl-N-(3-chloro-4-fluorophenyl)-DL-alanine, N-benzoyl-N-(3-chloro-4-fluorophenyl)-DL-alanine methyl ester, N-benzoyl-N-(3-chloro-4-fluorophenyl)-DL-alanine ethyl ester, N-benzoyl-N-(3-chloro-4-fluorophenyl)-D-alanine, N-benzoyl-N-(3-chloro-4-fluorophenyl)-D-alanine methyl ester, or N-benzoyl-N-(3-chloro-4-fluorophenyl)-D-alanine isopropyl ester.

The selection compound may be used in combination with other substances. For the purpose of application, the selection compound may also be used together with the adjuvants conventionally employed in the art of formulation, and are therefore formulated in known manner, e.g. into emulsifiable concentrates, coatable pastes, directly sprayable or dilutable solutions, dilute emulsions, wettable powders, soluble powders, dusts, granulates, and also encapsulations in e.g. polymer substances.

As with the nature of the compositions to be used, the methods of application, such as spraying, atomising, dusting, scattering, coating or pouring, are chosen in accordance with the intended objectives and the prevailing circumstances. However, more preferably the selection compound is directly applied to the medium. It is an advantage that stock solutions of the selection compound can be made and stored at room temperature for an extended period without a loss of selection efficiency.

The optimal concentration of the selection compound (i.e. D-alanine, D-serine, derivatives thereof or any combination thereof) may vary depending on the target tissue employed for transformation but in general (and preferably for immature embryo transformation) the total concentration (i.e. the sum in case of a mixture) of D-alanine, D-serine or derivatives thereof is in the range from about 1 mM to about 100 mM. For example in cases where the E. coli D-serine ammonia-lyase is employed, selection is done on a medium comprising D-serine (e.g., incorporated into agar-solidified MS media plates), preferably in a concentration from about 1 mM to about 100 mM, more preferably from about 2 mM to about 50 mM, even more preferably from about 3 mM to about 30 mM, most preferably about 5 mM to 10 mM. In cases where the Rhodotorula gracilis D-amino acid oxidase is employed, selection is done on a medium comprising D-alanine and/or D-serine (e.g., incorporated into agar-solidified MS media plates), preferably in a total concentration from about 1 mM to 100 mM, more preferably from about 2 mM to about 50 mM, even more preferably from about 3 mM to about 20 mM, most preferably about 5 mM to 10 mM.

Also the selection time may vary depending on the target tissue used and the regeneration protocol employed. In general a selection time is at least 14 days. More specifically the total selection time under dedifferentiating conditions (i.e., callus induction) is from about 1 to about 10 weeks, preferably, 3 to 9 weeks, more preferably 5 to 8 weeks. However, it is preferred that the selection under the dedifferentiating conditions is employed for not longer than 70 days. In between the selection period the callus may be transferred to fresh selection medium one or more times. For the specific protocol provided herein it is preferred that two selection medium steps (e.g., one transfer to new selection medium) is employed. Preferably, the selection of step is done in two steps, using a first selection step for about 5 to 35 days, then transferring the surviving cells or tissue to a second selection medium with essentially the same composition than the first selection medium for additional 5 to 25 days.

Preferably said selection medium is—for part of the selection period—also a dedifferentiation medium comprising at least one suitable plant growth regulator for induction of embryogenic callus formation. The term “plant growth regulator” (PGR) as used herein means naturally occurring or synthetic (not naturally occurring) compounds that can regulate plant growth and development. PGRs may act singly or in consort with one another or with other compounds (e.g., sugars, amino acids). More specifically the medium employed for embryogenic callus induction and selection comprises
i. an effective amount of at least one auxin compound, and
ii. an effective amount of a selection agent allowing for selection of cells comprising the transgenic.

Furthermore the embryogenic callus induction medium may optionally comprise an effective amount of at least one antibiotic that inhibits or suppresses the growth of the soil-borne bacteria (as defined above).

The term “auxin” or “auxin compounds” comprises compounds which stimulate cellular elongation and division, differentiation of vascular tissue, fruit development, formation of adventitious roots, production of ethylene, and—in high concentrations—induce dedifferentiation (callus formation). The most common naturally occurring auxin is indoleacetic acid (IAA), which is transported polarly in roots and stems. Synthetic auxins are used extensively in modern agriculture. Synthetic auxin compounds comprise indole-3-butyric acid (IBA), naphthylacetic acid (NAA), and 2,4-dichlorphenoxyacetic acid (2,4-D), Dicamba.

Preferably, in one embedment when used as the sole auxin compound, 2,4-D in a concentration of about 0.2 mg/l to about 6 mg/l, more preferably about 0.3 to about 5 mg/l, most preferably about 3 mg/l is employed. In case other auxin compounds or combinations thereof are employed, their preferred combinations is chosen in a way that the dedifferentiating effect is equivalent to the effect achieved with the above specified concentrations of 2,4-D when used as the sole auxin compound. Thus, the effective amount of the auxin compound is preferably equivalent to a concentration of about 0.2 mg/l to about 6 mg/l (more preferably about 0.3 to about 5 mg/l, most preferably about 3 mg/l) of 2,4-D.

Preferably in another embedment, when used as the sole auxin compound, Dicamba in a concentration of about 0.2 mg/l to about 6 mg/l, more preferably about 0.3 to about 5 mg/l, most preferably about 2.5 mg/l is employed. In case other auxin compounds or combinations thereof are employed, their preferred combinations is chosen in a way that the dedifferentiating effect is equivalent to the effect achieved with the above specified concentrations of Dicamba when used as the sole auxin compound. Thus, the effective amount of the auxin compound is preferably equivalent to a concentration of about 0.2 mg/l to about 6 mg/l (more preferably about 0.3 to about 2 mg/l, most preferably about 2.5 mg/l of Dicamba

Furthermore, combination of different auxins can be employed, for example a combination of 2,4-D and Picloram or Dicamba. Preferably, 2,4-D in a concentration of about 0.5 to 2 mg/l can be combined with one or more other types of auxin compounds e.g. Picloram in a concentration of about 0.5 to about 2.5 mg/l or/and Dicamba in concentration 0.5 to about 2.5 mg/l for improving quality/quantity of embryogenic callus formation.

The medium may be optionally further supplemented with one or more additional plant growth regulator, like e.g., cytokinin compounds (e.g., 6-benzylaminopurine) and/or other auxin compounds. Such compounds include, but are not limited to, IAA, NAA, IBA, cytokinins, auxins, kinetins, glyphosate, and thidiazuron. Cytokinin compounds comprise, for example zeatin, 6-isopentenyladenine (IPA) and 6-benzyladenine/6-benzylaminopurine (BAP).

The presence of the D-amino acid metabolizing enzymes does not rule out that additional markers are employed.

The selection (application of the selection compound) may end after the dedifferentiation and selection period. However, it is preferred to apply selection also during the subsequent regeneration period (in part or throughout), and even during rooting. In one typical selection scheme the following conditions may be applied:
Selection I: Selection under dedifferentiation conditions (callus proliferation) for about 7 to about 70 days, preferably from about 14 to about 50 days. Selection can be preferably done under light with a medium such as PAB-2 (see Example 3).
Selection II: Selection under regeneration conditions (see below) for about 7 to about 50 days, preferably for about 3 weeks (21 days). Regenerations can be done with a medium such as PAB-4 sel (see Example 3).
Selection III Selection under shoot elongation conditions for about 7 to about 50 days, preferably for about 3 weeks (21 days). Shoot elongation can be done with a medium such as PAB-5 selection in plates (see Examples).
Selection IV Selection under shoots growth and rooting conditions for about 7 to about 50 days, preferably for about 3 weeks (21 days). Shoots growth and rooting can be done with a medium such as PAB-5 selection in boxes (see Examples).

### 2.5 Regeneration

The formation of shoot and root from dedifferentiated cells can be induced in the known fashion. The shoots obtained can be planted and cultured. Transformed barley plant cells, preferably barley embryogenic cells derived by any of the above transformation techniques, can be cultured to regenerate a whole plant which possesses the transformed genotype and thus the desired phenotype. Such regeneration techniques rely on manipulation of certain phytohormones in a tissue culture growth medium. Plant regeneration from cultured protoplasts is described (e.g. Lazzeri et al. 1991). Regeneration can also be obtained from protoplast derived callus, microspores, axis of immature embryos (Kihara et al. 1998; Wan and Lemaux 1994; Ritalla et al. 1994). Other available regeneration techniques are reviewed in Lemaux et al. 1999.

After the dedifferentiation and selection period (as described above) the resulting cells (e.g., maturing embryogenic callus) are transferred to a medium allowing conversion of transgenic plantlets. Preferably such medium does not comprise auxins such as 2,4-D in a concentration leading to dedifferentiation. In a preferred embodiment such medium may comprise one or more compounds selected from the group consisting of:
i) cytokinins such as for example 6-benzyladenine/6-benzylaminopurine (BAP) preferably in a concentration from about 0.5 to about 10 mg/L, more preferably from about 1.0 to about 5 mg/L,
ii) an effective amount of at least one antibiotic that inhibits or suppresses the growth of the soil-borne bacteria (as defined above), and
iii) an effective amount of a selection agent (e.g., D-alanine, D-serine, or derivatives thereof) allowing for selection of transgenic cells (e.g., comprising the transgenic T-DNA).

The embryogenic callus is preferably incubated on this medium until shoots are formed and then transferred to a elongation hormone free medium. Such incubation may take from 1 to 5, preferably from 2 to 3 weeks. Regenerated shoots or plantlets (i.e., shoots with roots) are transferred to Phytatray, Magenta boxes or Sky-Light plastic boxes containing rooting medium (such as the medium described in PAB-5) and incubate until rooted plantlets have developed (usually 1 to 4 weeks, preferably 2 weeks). The rooted seedlings are transferred to Jiffy for aclimatisation (usually for 10 days) After analyses the transgenic plants are transferred to soil K-Jord and grown to mature plants as described in the art (see Examples).

The resulting transgenic plants are self pollinated by bagging all spikes individually while they are emerging from the flag leaf. T1 seeds are spikewise harvested, dried and stored properly with adequate label on the seed bags. Two or more generations should be grown in order to ensure that the genomic integration is stable and hereditary For example transgenic events in T1 or T2 generations could be involved in pre breeding hybridization program for combining different transgenes (gene stucking).

Other important aspects of the invention include the progeny of the transgenic plants prepared by the disclosed methods, as well as the cells derived from such progeny, and the seeds obtained from such progeny.

### 2.6 Generation of Descendants

After transformation, selection and regeneration of a transgenic plant (comprising the DNA construct of the invention) descendants are generated, which—because of the activity of the excision promoter—underwent excision and do not comprise the marker sequence(s) and expression cassette for the endonuclease.

Descendants can be generated by sexual or non-sexual propagation. Non-sexual propagation can be realized by introduction of somatic embryogenesis by techniques well known in the art. Preferably, descendants are generated by sexual propagation/fertilization. Fertilization can be realized either by selfing (self-pollination) or crossing with other transgenic or non-transgenic plants. The transgenic plant of the invention can herein function either as maternal or paternal plant.

After the fertilization process, seeds are harvested, germinated and grown into mature plants. Isolation and identification of descendants which underwent the excision process can be done at any stage of plant development. Methods for said identification are well known in the art and may comprise—for example—PCR analysis, Northern blot, Southern blot, or phenotypic screening (e.g., for an negative selection marker).

Descendants may comprise one or more copies of the agronomically valuable trait gene. Preferably, descendants are isolated which only comprise one copy of said trait gene.

Also in accordance with the invention are cells, cell cultures, parts—such as, for example, in the case of transgenic plant organisms, roots, leaves and the like—derived from the above-described transgenic organisms, and transgenic propagation material (such as seeds or fruits).

Genetically modified plants according to the invention which can be consumed by humans or animals can also be used as food or feedstuffs, for example directly or following processing known per se. Here, the deletion of, for example, resistances to antibiotics and/or herbicides, as are frequently introduced when generating the transgenic plants, makes sense for reasons of customer acceptance, but also product safety.

A further subject matter of the invention relates to the use of the above-described transgenic organisms and the cells, cell cultures, and/or parts—such as, for example, in the case of transgenic plant organisms, roots, leaves and the like—derived from them, and transgenic propagation material such as seeds or fruits, for the production of food or feedstuffs, pharmaceuticals or fine chemicals.

A further subject matter of the invention relates to a composition for selection, regeneration, growing, cultivation or maintaining of transgenic barley plant cells, transgenic barley plant tissue, transgenic barley plant organs or transgenic barley plants or a part thereof comprising an effective amount of D-alanine, D-serine, or a derivative thereof allowing for selection of transgenic barley plant cells, transgenic barley plant tissue, transgenic barley plant organs or transgenic barley plants or a part thereof and the above-described transgenic barley organisms, the transgenic barley cells, transgenic barley cell cultures, transgenic barley plants and/or parts thereof—such as, for example, in the case of transgenic plant organisms roots, leaves and the like—derived from them.

Another embodiment of the invention relates to a barley plant or cell comprising a promoter active in said barley plants or cells and operably linked thereto a nucleic acid sequence encoding an enzyme capable to metabolize D-alanine or D-serine, wherein said promoter is heterologous in relation to said enzyme encoding sequence. Preferably, the promoter and/or the enzyme capable to metabolize D-alanine or D-serine is defined as above. More preferably the barley plant is further comprising at least one second expression construct conferring to said barley plant an agronomically valuable trait. In one preferred embodiment the barley plant selected from the Triticum family group of plants. More preferably from a plant specie of the group consisting of Hordeum (H. vulgare subsp. Vulgare and Hordeum vulgare subsp. Spontaneum all diploid and tetraploid forms).

Other embodiments of the invention relate to parts, organs, cells, fruits, and other reproduction material of a barley plant of the invention. Preferred parts are selected from the group consisting of tissue, cells, pollen, ovule, roots, leaves, seeds, microspores, and vegetative parts.

Fine chemicals is understood as meaning enzymes, vitamins, amino acids, sugars, fatty acids, natural and synthetic flavors, aromas and colorants. Especially preferred is the production of tocopherols and tocotrienols, and of carotenoids. Culturing the transformed host organisms, and isolation from the host organisms or from the culture medium, is performed by methods known to the skilled worker. The production of pharmaceuticals such as, for example, antibodies or vaccines, is described (e.g., by Hood 1999; Ma 1999).

### 3. Further Modifications

### 3.1 Counter Selection and Subsequent Marker Deletion

The first expression construct for the D-amino acid metabolizing enzyme can be preferably constructed in a way to allow for subsequent marker deletion, especially when said enzyme is a D-amino acid oxidase, which can be employed both for negative selection and counter selection (i.e. as a dual-function marker). Such methods are in detail described in (ADD) hereby incorporated entirely by reference.

For this purpose the first expression cassette is preferably flanked by sequences, which allow for specific deletion of said first expression cassette. This embodiment of the invention makes use of the property of D-amino acid oxidase (DAAO) to function as dual-function markers, i.e., as markers which both allow (depending on the used substrate) as negative selection marker and counter selection marker. In contrast to D-amino acids like D-serine and D-alanine (which are highly phytoptoxic to plants and are “detoxified” by the D-amino acid oxidase), D-valine and D-isoleucine are not toxic to wild-type plants but are converted to toxic compounds by plants expressing the D-amino acid oxidase DAAO. The findings that DAAO expression mitigated the toxicity of D-serine and D-alanine, but induced metabolic changes that made D-isoleucine and D-valine toxic, demonstrate that the enzyme could provide a substrate-dependent, dual-function, selectable marker in plants.

Accordingly, another embodiment of the invention relates to a method for producing a transgenic barley plant comprising:
i) transforming a barley plant cell with a first DNA construct comprising
   a) at least one first expression construct comprising a promoter active in said barley plant and operably linked thereto a nucleic acid sequence encoding a D-amino acid oxidase enzyme, wherein said first expression cassette is flanked by sequences which allow for specific deletion of said first expression cassette, and
   b) at least one second expression cassette suitable for conferring to said plant an agronomically valuable trait, wherein said second expression cassette is not localized between said sequences which allow for specific deletion of said first expression cassette, and
ii) treating said transformed barley plant cells of step i) with a first compound selected from the group consisting of D-alanine, D-serine or derivatives thereof in a phytotoxic concentration and selecting plant cells comprising in their genome said first DNA construct, conferring resistance to said transformed plant cells against said first compound by expression of said D-amino acid oxidase, and
iii) inducing deletion of said first expression cassette from the genome of said transformed plant cells and treating said plant cells with a second compound selected from the group consisting of D-isoleucine, D-valine and derivatives thereof in a concentration toxic to plant cells still comprising said first expression cassette, thereby selecting plant cells comprising said second expression cassette but lacking said first expression cassette.

Preferred promoters and D-amino acid oxidase sequences are described above.

Preferably, deletion of the first expression cassette can be realized by various means known in the art, including but not limited to one or more of the following methods:
a) recombination induced by a sequence specific recombinase, wherein said first expression cassette is flanked by corresponding recombination sites in a way that recombination between said flanking recombination sites results in deletion of the sequences in-between from the genome,
b) homologous recombination between homology sequences A and A′ flanking said first expression cassette, preferably induced by a sequence-specific double-strand break between said homology sequences caused by a sequence specific endonuclease, wherein said homology sequences A and A′ have sufficient length and homology in order to ensure homologous recombination between A and A′, and having an orientation which—upon recombination between A and A′—will lead to excision of said first expression cassette from the genome of said plant.

Various means are available for the person skilled in art to combine the deletion/excision inducing mechanism with the DNA construct of the invention comprising the D-amino acid oxidase dual-function selection marker. Preferably, a recombinase or endonuclease employable in the method of the invention can be expressed by a method selected from the group consisting of:
a) incorporation of a second expression cassette for expression of the recombinase or sequence-specific endonuclease operably linked to a plant promoter into said DNA construct, preferably together with said first expression cassette flanked by said sequences which allow for specific deletion,
b) incorporation of a second expression cassette for expression of the recombinase or sequence-specific endonuclease operably linked to a plant promoter into the plant cells or plants used as target material for the transformation thereby generating master cell lines or cells,
c) incorporation of a second expression cassette for expression of the recombinase or sequence-specific endonuclease operably linked to a plant promoter into a separate DNA construct, which is transformed by way of co-transformation with said first DNA construct into said plant cells,
d) incorporation of a second expression cassette for expression of the recombinase or sequence-specific endonuclease operably linked to a plant promoter into the plant cells or plants which are subsequently crossed with plants comprising the DNA construct of the invention.

In another preferred embodiment the mechanism of deletion/excision can be induced or activated in a way to prevent pre-mature deletion/excision of the dual-function marker. Preferably, thus expression and/or activity of an preferably employed sequence-specific recombinase or endonuclease can be induced and/or activated, preferably by a method selected from the group consisting of
a) inducible expression by operably linking the sequence encoding said recombinase or endonuclease to an inducible promoter,
b) inducible activation, by employing a modified recombinase or endonuclease comprising a ligand-binding-domain, wherein activity of said modified recombinase or endonuclease can by modified by treatment of a compound having binding activity to said ligand-binding-domain.

Preferably, thus the method of the inventions results in a plant cell or plant which is selection marker-free.

Another subject matter of the invention relates to DNA constructs, which are suitable for employing in the method of the invention. A DNA construct suitable for use within the present invention is preferably comprising
a) a first expression cassette comprising a nucleic acid sequence encoding a D-amino acid oxidase operably linked with a promoter active in barley plants (as defined above; preferably an ubiquitin promoter), wherein said first expression cassette is flanked by sequences which allow for specific deletion of said first expression cassette, and
b) at least one second expression cassette suitable for conferring to said plant an agronomically valuable trait, wherein said second expression cassette is not localized between said sequences which allow for specific deletion of said first expression cassette.

Preferred promoters and D-amino acid oxidase sequences are described above.

For ensuring marker deletion/excision the expression cassette for the D-amino acid oxidase (the first expression construct) comprised in the above mentioned DNA construct is flanked by recombination sites for a sequence specific recombinase in a way the recombination induced between said flanking recombination sites results in deletion of the said first expression cassette from the genome. Preferably said sequences which allow for specific deletion of said first expression cassette are selected from the group of sequences consisting of
a) recombination sites for a sequences-specific recombinase arranged in a way that recombination between said flanking recombination sites results in deletion of the sequences in-between from the genome, and
b) homology sequences A and A′ having a sufficient length and homology in order to ensure homologous recombination between A and A′, and having an orientation which—upon recombination between A and A′—results in deletion of the sequences in-between from the genome.

Preferably, the construct comprises at least one recognition site for a sequence specific nuclease localized between said sequences that allow for specific deletion of said first expression cassette (especially for variant b above).

There are various recombination sites and corresponding sequence specific recombinases known in the art, which can be employed for the purpose of the invention. The person skilled in the art is familiar with a variety of systems for the site-directed removal of recombinantly introduced nucleic acid sequences. They are mainly based on the use of sequence specific recombinases. Various sequence-specific recombination systems are described, such as the Cre/lox system of the bacteriophage P1 (Dale 1991; Russell 1992; Osborne 1995), the yeast FLP/FRT system (Kilby 1995; Lyznik 1996), the Mu phage Gin recombinase, the E. coli Pin recombinase or the R/RS system of the plasmid pSR1 (Onouchi 1995; Sugita 2000). Also a system based on attP sites and bacteriophage Lambda recombinase can be employed (Zubko 2000). Further methods suitable for combination with the methods described herein are described in WO 97/037012 and WO 02/10415.

In a preferred embodiment, deletion/excision of the dual-marker sequence is deleted by homologous recombination induced by a sequence-specific double-strand break. The basic principles are disclosed in WO 03/004659, hereby incorporated by reference. For this purpose the first expression construct (encoding for the dual-function marker) is flanked by homology sequences A and A′, wherein said homology sequences have sufficient length and homology in order to ensure homologous recombination between A and A′, and having an orientation which—upon recombination between A and A′—will lead to an excision of first expression cassette from the genome. Furthermore, the sequence flanked by said homology sequences further comprises at least one recognition sequence of at least 10 base pairs for the site-directed induction of DNA double-strand breaks by a sequence specific DNA double-strand break inducing enzyme, preferably a sequence-specific DNA-endonuclease, more preferably a homing-endonuclease, most preferably an endonuclease selected from the group consisting of I-SceI, I-CeuI, I-CpaI, I-CpaII, I-CreI and I-ChuI or chimeras thereof with ligand-binding domains.

The expression cassette for the endonuclease or recombinase (comprising a sequence-specific recombinase or endonuclease operably linked to a plant promote) may be included in the DNA construct of the invention. Preferably, said second expression cassette is together with said first expression cassette flanked by said sequences which allow for specific deletion.

In another preferred embodiment, the expression and/or activity of said sequence-specific recombinase or endonuclease can be induced and/or activated for avoiding premature deletion/excision of the dual-function marker during a period where its action as a negative selection marker is still required. Preferably induction/activation can be realized by a method selected from the group consisting of
a) inducible expression by operably linking the sequence encoding said recombinase or endonuclease to an inducible promoter,
b) inducible activation, by employing a modified recombinase or endonuclease comprising a ligand-binding-domain, wherein activity of said modified recombinase or endonuclease can by modified by treatment of a compound having binding activity to said ligand-binding-domain.

Further embodiments of the inventions are related to transgenic vectors comprising a DNA construct of the invention. Transgenic cells or non-human organisms comprising a DNA construct or vector of the invention. Preferably said cells or non-human organisms are plant cells or plants, preferably plants which are of agronomical use.

The present invention enables generation of marker-free transgenic cells and organisms, preferably plants, an accurately predictable manner with high efficiency.

The preferences for the counter selection step (ii) with regard to choice of compound, concentration, mode of application for D-alanine, D-serine, or derivatives thereof are described above in the context of the general selection scheme.

For the counter selection step (iii) the compound is selected from the group of compounds comprising a D-isoleucine or D-valine structure. More preferably the compound is selected from the group consisting of D-isoleucine and D-valine. Most preferably the compound or composition used for counter selection comprises D-isoleucine.

When applied via the cell culture medium (e.g., incorporated into agar-solidified MS media plates), D-isoleucine can be employed in concentrations of about 0.5 mM to about 100 mM, preferably about 1 mM to about 50 mM, more preferably about 10 mM to about 30 mM. When applied via the cell culture medium (e.g., incorporated into agar-solidified MS media plates), D-valine can be employed in concentrations of about 1 to about 100 mM, preferably about 5 to 50 mM, more preferably about 15 mM to about 30 mM.

Thus, using the above described method it becomes possible to create a barley plant which is marker-free. The terms “marker-free” or “selection marker free” as used herein with respect to a cell or an organisms are intended to mean a cell or an organism which is not able to express a functional selection marker protein (encoded by expression cassette b; as defined above) which was inserted into said cell or organism in combination with the gene encoding for the agronomically valuable trait. The sequence encoding said selection marker protein may be absent in part or—preferably—entirely. Furthermore the promoter operably linked thereto may be dysfunctional by being absent in part or entirely. The resulting plant may however comprise other sequences which may function as a selection marker. For example the plant may comprise as a agronomically valuable trait a herbicide resistance conferring gene. However, it is most preferred that the resulting plant does not comprise any selection marker.

Various further aspects and embodiments of the present invention will be apparent to those skilled in the art in view of the present disclosure. All documents mentioned in this specification are incorporated herein in their entirety by reference. Certain aspects and embodiments of the invention will now be illustrated by way of example and with reference to the figure described below.

### 3.2 Gene Stacking

The methods and compositions of the invention allow for subsequent transformation. The D-serine and/or D-alanine metabolizing enzymes are compatible and does not interfere with other selection marker and selection systems. It is therefore possible to transform existing transgenic plants comprising another selection marker with the constructs of the invention or to subsequently transform the plants obtained by the method of the invention (and comprising the expression constructs for the D-serine and/or D-alanine metabolizing enzyme) with another marker. This, another embodiment of the invention relates to a method for subsequent transformation of at least two DNA constructs into a barley plant comprising the steps of:
a) a transformation with a first construct said construct comprising at least one expression construct comprising a promoter active in said barley plants and operably linked thereto a nucleic acid sequence encoding an enzyme capable to metabolize D-alanine or D-serine, and
b) a transformation with a second construct said construct comprising a second selection marker gene, which is not conferring resistance against D-alanine or D-serine.

Preferably said second marker gene is a negative selection markers conferring a resistance to a biocidal compound such as a (non-D-amino acid) metabolic inhibitor (e.g., 2-deoxyglucose-6-phosphate, WO 98/45456), antibiotics (e.g., kanamycin, G 418, bleomycin or hygromycin) or herbicides (e.g., phosphinothricin or glyphosate). Examples are:
Phosphinothricin acetyltransferases (PAT; also named Bialophos® resistance; bar; de Block 1987; Vasil 1992, 1993; Weeks 1993; Becker 1994; Nehra 1994; Wan & Lemaux 1994; EP 0 333 033; U.S. Pat. No. 4,975,374)
5-enolpyruvylshikimate-3-phosphate synthase (EPSPS) conferring resistance to Glyphosate® (N-(phosphonomethyl)glycine) (Shah 1986; Della-Cioppa 1987)
Glyphosate® degrading enzymes (Glyphosate® oxidoreductase; gox),
Dalapon® inactivating dehalogenases (deh)
sulfonylurea- and/or imidazolinone-inactivating acetolactate synthases (ahas or ALS; for example mutated ahas/ALS variants with, for example, the S4, XI12, XA17, and/or Hra mutation
Bromoxynil® degrading nitrilases (bxn)
Kanamycin- or. geneticin (G418) resistance genes (NPTII; NPTI) coding e.g., for neomycin phosphotransferases (Fraley 1983; Nehra 1994)
hygromycin phosphotransferase (HPT), which mediates resistance to hygromycin (Vanden Elzen 1985).
dihydrofolate reductase (Eichholtz 1987)

Various time schemes can be employed for the various negative selection marker genes. In case of resistance genes (e.g., against herbicides) selection is preferably applied throughout callus induction phase for about 4 weeks and beyond at least 4 weeks into regeneration. Such a selection scheme can be applied for all selection regimes. It is furthermore possible (although not explicitly preferred) to remain the selection also throughout the entire regeneration scheme including rooting. For example, with the phosphinotricin resistance gene (bar, PAT) as the selective marker, phosphinotricin or bialaphos at a concentration of from about 1 to 50 mg/l may be included in the medium.

Preferably, said second marker gene is defined as above and is most preferably conferring resistance against at least one compound select from the group consisting of phosphinotricin, glyphosate, phosphinotricin, glyphosate, sulfonylurea- and imidazolinone-type herbicides.

The following combinations are especially preferred:
1. A first transformation with an pat,bar selection marker gene followed by a second transformation with a dsdA selections marker gene;
2. A first transformation with an pat/bar selection marker gene followed by a second transformation with a dao1 selection marker gene;
3. A first transformation with a dsdA selection marker gene followed by a second transformation with an pat/bar selection marker gene;
4. A first transformation with a dao1 followed by a second transformation with an pat/bar selection marker gene;

Beside the stacking with a second expression construct for a selection marker gene, which is not conferring resistance against D-alanine or D-serine, also the dsdA and dao1 genes can be stacked. For example a first selection can be made using the dsdA gene and D-serine as a selection agent and a second selection can be subsequently made by using dao1 gene and D-alanine as selection agent. Thus another embodiment of the invention relates to a method for subsequent transformation of at least two DNA constructs into a barley plant comprising the steps of:
a) a transformation with a first construct said construct comprising an expression construct comprising a promoter active in said barley plants (preferably a ubiquitin promoter as defined above) and operably linked thereto a nucleic acid sequence encoding a dsdA enzyme and selecting with D-serine, and
b) a transformation with a second construct said construct comprising an expression construct comprising a promoter active in said barley plants and operably linked thereto a nucleic acid sequence encoding a dao enzyme and selecting with D-alanine.

Another embodiment of the invention relates to the barley plants generated with this method. Thus, the invention also relates to a barley plant comprising
a) a first construct said construct comprising an expression construct comprising a promoter active in said barley plants and operably linked thereto a nucleic acid sequence encoding an dsdA enzyme, and
b) a second construct said construct comprising an expression construct comprising promoter active in said barley plants and operably linked thereto a nucleic acid sequence encoding a dao enzyme.

In the above mentioned constructs comprising two expression cassettes it is preferred that the two promoters active in barley plants are not identical. Preferably one promoter (e.g., the promoter for expression of the D-alanine and/or D-serine metabolizing enzyme) is an ubiquitin promoter as defined above), while the other promoter is a different promoter (e.g., the ScBV promoter or the ahas promoter).

### Sequences

1. SEQ ID NO: 1 Nucleic acid sequence encoding E. coli D-serine dehydratase [dsdA] gene
2. SEQ ID NO: 2 Amino acid sequence encoding E. coli D-serine dehydratase [dsdA]
3. SEQ ID NO: 3 Nucleic acid sequence encoding Rhodosporidium toruloides D-amino acid oxidase gene
4. SEQ ID NO: 4 Amino acid sequence encoding Rhodosporidium toruloides D-amino acid oxidase
5. SEQ ID NO: 5 Nucleic acid sequence encoding maize ubiquitin core promoter region
6. SEQ ID NO: 6 Nucleic acid sequence encoding maize ubiquitin promoter further comprising 5′-untranslated region and first intron
7. SEQ ID NO: 7 Nucleic acid sequence encoding sugarcane bacilliform virus core promoter region
8. SEQ ID NO: 8 Nucleic acid sequence encoding sugarcane bacilliform virus promoter further comprising 5′-untranslated region
9. SEQ ID NO:9 Nucleic acid sequence encoding pRLM175, a kanamycin resistant SB11-type binary vector.
10. SEQ ID NO:10 Nucleic acid sequence encoding T-DNA region of pRLM166, a pRLM175 derived binary vector containing p-ZmUBI+l::c-dsdA::t-OCS and p-ScBV::c-gusINT::t-NOS cassettes.
11. SEQ ID NO:11 Nucleic acid sequence encoding T-DNA region of pRLM167, a pRLM175 derived binary vector containing p-ZmUBI+l::c-dsdA::t-OCS and p-ZmUBI+l::c-PAT::t-OCS cassettes.
12. SEQ ID NO:12 Nucleic acid sequence encoding T-DNA region of pRLM205, a pRLM175 derived binary vector containing p-ZmUBI+l::c-dao1::t-OCS and p-ScBV::c-gusINT::t-NOS cassettes.
13 SEQ ID NO:13 Nucleic acid sequence encoding qPCR primer GUSCommon-341F:

5′ CCGGGTGAAG GTTATCTCTA TGA 3′

14 SEQ ID NO:14 Nucleic acid sequence encoding qPCR primer GUSCommon-414R:

5′ CGAAGCGGGT AGATATCACA CTCT 3′

15. SEQ ID NO:15 Nucleic acid sequence encoding qPCR probe GUSCommon-366FAM:

5′ TGTGCGTCAC AGCCAAAAGC CAGA 3′

16. SEQ ID NO:16 Nucleic acid sequence encoding qPCR primer EcdsdA-860F:

5′ TCGCATTCGG GCTTAAACTG 3′

17. SEQ ID NO: 17 Nucleic acid sequence encoding qPCR primer EcdsdA-922R:

5′ GCGTTGGTTC GGCAAAAA 3′

18. SEQ ID NO: 18 Nucleic acid sequence encoding qPCR probe EcdsdA-883FAM:

5′ TTTGGCGATC ATGTTCACTG C 3′

19. SEQ ID NO: 19 Nucleic acid sequence encoding qPCR primer dao1/pa-285F:

5′ GTTCGCGCAG AACGAAGAC 3′

20. SEQ ID NO: 20 Nucleic acid sequence encoding qPCR primer dao1/pa-349R:

5′ GGCGGTAATT TGGCGTGA 3′

21. SEQ ID NO: 21 Nucleic acid sequence encoding qPCR probe dao1/pa-308FAM:

5′ TCCTTGTACC AGTGCCCGAG CA 3′

22. SEQ ID NO: 22 Nucleic acid sequence encoding forward PCR primer for gu-sINT gene:

5′-ACCGTTTGTG TGAACAACGA -3′

23. SEQ ID NO: 23 Nucleic acid sequence encoding reverse PCR primer for gu-sINT gene:

5′- GGCACAGCAC ATCAAAGAGA- 3′

24. SEQ ID NO: 24 Nucleic acid sequence encoding forward PCR primer for dsdA gene:

5′-GCTTTTTGTT CGCTTGGTTG TG -3′

25. SEQ ID NO: 25 Nucleic acid sequence encoding reverse PCR primer for dsdA gene:

5′-TCAATAATCC CCCCAGTGGC- 3′

26. SEQ ID NO: 26 Nucleic acid sequence encoding forward PCR primer for dao1 gene:

5′-GACAAGCAAA ATGGGAAGAA TC -3′

27. SEQ ID NO: 27 Nucleic acid sequence encoding reverse PCR primer for dao1 gene:

5′-TCGGGGAATG ATGTAGGC -3′

28. SEQ ID NO: 28 Nucleic acid sequence encoding forward PCR primer for PAT gene:

5′- ATGTCTCCGGAGAGGAGACCAGTTGAGAT-3′

29. SEQ ID NO: 29 Nucleic acid sequence encoding reverse PCR primer for PAT gene:

5′- GCCAAAAACCAACATCATGCCATCCA-3′

### EXAMPLES

### General Methods

Unless indicated otherwise, chemicals and reagents in the Examples were obtained from Sigma-Aldrich AB, Sweden Materials for cell culture media were obtained from GIBCO Invitrogene AB Sweden Duchefa SAVEEN, Sweden or DIFCO Nordica Biolabs, Sweden. The cloning steps carried out for the purposes of the present invention, such as, for example, transformation of E. coli cells, growing bacteria, multiplying phages and sequence analysis of recombinant DNA, are carried out as described by Sambrook (1989). The following examples are offered by way of illustration and not by way of limitation.

### Medium for Transformation

TABLE 2
Composition of the PAW set of media used in Example 2

| | |
|---|---|
| PAW-Infection | MS micro, macro salts, 4.3 g l⁻¹, Nicotinic acid 0.5 mg l⁻¹ |
| medium | Pyridoxine HCl 0.5 mg l⁻¹, Thiamin HCl 1.0 mg l⁻¹, Myo- |
| | inositol 0.1 g l⁻¹, Casamino acid 1.0 g l⁻¹, 2,4-D 2.0 mg l⁻¹, |
| | Sucrose 68.46 g (0.2M), Glucose 39.63 g (0.2M); |
| | pH = 5.2; Compound added: Acetosyringone (300 μM) |
| PAW-1 | MS micro, macro salts 4.3 g l⁻¹, Nicotinic acid 0.5 mg l⁻¹, |
| Co-cultivation | Pyridoxine HCl 0.5 mg l⁻¹, Thiamin HCl 1.0 mg l⁻¹, Myo- |
| medium | inositol 0.1 g l⁻¹, Glutamine 0.5 g l⁻¹, Casein hydrolysate |
| | 0.1 g l⁻¹, Ascorbic acid 0.1 g l⁻¹, CuSO₄ × 5H₂O 0.5 mg l⁻¹, |
| | MES 0.5 g l⁻¹, 2.4-D 2.0 mg l⁻¹, Sucrose 20 g l⁻¹, Malt- |
| | ose 10 g l⁻¹, Glucose 10 g l⁻¹, Gelrite 2.5 g l⁻¹; pH = 5.65; |
| | Compound added: Acetosyringone (300 μM) |
| PAW-2 | PAW-1 composition pH = 5.65; Compounds added: Ti- |
| Callus Induction | mentin 160 mg l⁻¹ |
| Recovery medium | |
| PAW-2 | MS macro, micro salts 4.3 g l⁻¹, Nicotinic acid 0.5 mg l⁻¹, |
| Callus Proliferation | Pyridoxine HCl 0.5 mg l, Thiamin HCl 1.0 mg l, Myo- |
| Selection medium | inositol 0.1 g l⁻¹, Glutamine 0.5 g l⁻¹, Casein hydrolysate |
| | 0.1 g l⁻¹, Ascorbic acid 0.1 g l, CuSO₄ × 5H₂O 0.5 mg l, |
| | MES 0.5 g l, 2,4-D 2.0 mg l⁻¹, Sucrose 20 g l⁻¹, Maltose |
| | 10 g l⁻¹, Gelrite 2.5 g l⁻¹; pH = 5.65; Compounds added: |
| | Timentin 160 mg l⁻¹, D-Serine (5 mM), |
| PAW-4 | MS macro, micro salts 4.3 g l⁻¹, Nicotinic acid 0.5 mg l⁻¹, |
| Regeneration | Pyridoxine HCl 0.5 mg l, Thiamin HCl 1.0 mg l, Myo- |
| medium | inositol 0.1 g l⁻¹, CuSO₄ × 5H₂O 0.5 mg l⁻¹, MES 0.5 g l⁻¹, |
| | Sucrose 20 g l⁻¹, |
| | Maltose 10 g l⁻¹, Gelrite 2.5 g l⁻¹, Zeatin 5.0 mg l⁻¹, |
| | Gelrite 2.5 g l⁻¹; pH = 5.65; |
| | Compounds added: Timentin 160 mg/l⁻¹, D-Serine (5 |
| | mM) |
| PAW-5 Medium for | MS macro, micro salts 2.15 g l⁻¹, Nicotinic acid 0.5 mg l⁻¹, |
| Shoots Elongation, | Pyridoxine HCl 0.5 mg/l¹, Thiamin HCl 1.0 mg l⁻¹, |
| Rooting and Embryos | Myo-inositol 0.1 g l⁻¹, MES 0.5 g l⁻¹, Sucrose 20 g l⁻¹, |
| Germination | Gelrite 2.5 g l⁻¹, pH = 5.65; |
| | Compounds added: Timentin 160 mg l⁻¹, D-Serine (5 mM |

TABLE 3
Composition of the PAB set of media used in Example 3

| | |
|---|---|
| PAB-Infection | 1/10MS micro, macro salts, 4.3 g l⁻¹, Myo-inositol 0.1 g l⁻¹, |
| medium | Casamino acid 1.0 g l⁻¹, 2,4-D 2.0 mg l⁻¹, Glucose 20 |
| | g, pH = 5.2; |
| | Compound added: Acetosyringone (300 μM) |
| PAB-1 | MS micro, macro salts 4.3 g l⁻¹, Nicotinic acid 0.5 mg l⁻¹, |
| Co-cultivation | Pyridoxine HCl 0.5 mg l⁻¹, Thiamin HCl 1.0 mg l⁻¹, Myo- |
| medium | inositol 0.5 g l⁻¹, L-Proline 690 mg l⁻¹, Casein hydrolysate |
| | 1 g l⁻¹, Ascorbic acid 0.1 g l⁻¹, CuSO₄ × 5H₂O 0.5 mg l⁻¹, |
| | MES 0.5 g l⁻¹, |
| | Dicamba 2.5 mg l⁻¹, Maltose 30 g l⁻¹, Gelrite 3.5 g l⁻¹; |
| | pH = 5.8 |
| | Compound added: Acetosyringone (300 μM) |
| PAB-2 | PAB-1 composition |
| Callus Induction | Compounds added: Timentin 160 mg l⁻¹ |
| Recovery medium | |
| PAB-3 | PAB-1 composition |
| Callus Proliferation | Compounds added: Timentin 160 mg l⁻¹, D-Serine 5 |
| Selection medium | mM, bialaphos 5 mg/l |
| PAB-4 | MS macro, micro salts 4.3 g l⁻¹, Nicotinic acid 0.5 mg l⁻¹, |
| Regeneration | Pyridoxine HCl 0.5 mg l, Thiamin HCl 1.0 mg l, Myo- |
| medium | inositol 0.1 g l⁻¹, L-Proline 690 mg l⁻¹, CuSO4 × 5H2O 0.5 |
| | mg l⁻¹, MES 0.5 g l⁻¹, Maltose 30 g l⁻¹, Gelrite 3.5 g l⁻¹, |
| | BAP 1.0 mg l⁻¹, Gelrite 3.5 g l⁻¹; pH = 5.8; |
| | Compounds added: Timentin 160 mg/l⁻¹, D-Serine 5 |
| | mM, bialaphos 1 mg/l |
| PAB-5 Medium for | MS macro, micro salts 2.15 g l⁻¹, Nicotinic acid 0.5 mg l⁻¹, |
| Shoots Elongation, | Pyridoxine HCl 0.5 mg/l¹, Thiamin HCl 1.0 mg l⁻¹, |
| Rooting and Embryos | Myo-inositol 0.1 g l⁻¹, MES 0.5 g l⁻¹, Sucrose 20 g l⁻¹, |
| Germination | Gelrite 2.5 g l⁻¹, pH = 5.65; |
| | Compounds added: Timentin 160 mg l⁻¹, D-Serine 5 |
| | mM, bialaphos 3 mg/l |

### Constructs

Following constructs were tested in barley transformation experiments (Table 4).

TABLE 4
Description of transformation vectors used for the experiments in establishing
transformation with dsdA and dao1 genes as the selection marker.

| | | Reporter/Selection | SEQ |
|---|---|---|---|
| Vector | LB-Selection marker | marker-RB | ID NO: |
| PRLM166 | p-ZmUBI + I::c-dsdA::t-OCS | p-ScBV::c-gusINT::t-NOS | 10 |
| PRLM167 | p-ZmUBI + I::c-dsdA::t-OCS | p-ZmUBI + I::c-PAT::t-OCS | 11 |
| PRLM205 | p-ZmUBI + I::c-dao1::t-OCS | p-ScBV::c-gusINT::t-NOS | 12 |

(EcdsdA = E. coli dsdA; dao1 = D- Amino acid oxydase gene; p-ScBV = ScBV promoter; p-ZmUbi = maize ubi promoter; t-OCS′ = OCS′ terminator; t-NOS = nos terminator; PsFed1 = translational leader sequence)

### Barley DNA Isolation and Analyses

Leaf material was collected in 96 format plates, freeze dried and DNA was extracted using Wizard Magnetic 96 DNA plant system (Promega, Cat NoFF3760). PCR reactions were performed using primers designed to amplify a 700 bp dsdA fragment, a 1000 bp gusINT fragment, a 485 bp dao1 fragment and 442 bp PAT fragment. Multiplex PCR for detecting simultaneously both transgenes was established. Reaction conditions were as following: Amplification of dsdA-gisINT fragments from pRLM166: “hot start” (95° C. 5 min) followed by 30 cycles of denaturation (94° C. 30 msec), annealing (62° C. 30 sec), extension (72° C. 30 sec) followed by 1 cycle of 72 (5 min) and then held at 4° C.

Amplification of dsdA-PAT and Dao1-gusINT fragments from pRLM167 and pRLM205: “hot start” (95° C. 5 min) followed by 30 cycles of denaturation (94° C. 30 msec), annealing (63° C. 30 sec), extension (72° C. 30 sec) followed by 1 cycle of 72 (5 min) and then held at 4° C.

Primarily transgenic plants were additionally evaluated for gene integration using real-time PCR TaqMan chemistry and specific primers and probes for the transgenes

### Real-Time PCR Primers/Probes: QPCR Primers/Probes

GUSCommon-341F

(SEQ ID NO: 13)

5′ CCGGGTGAAGGTTATCTCTATGA 3′
GUSCommon-414R

(SEQ ID NO: 14)

5′ CGAAGCGGGTAGATATCACACTCT 3′
GUSCommon-366FAM

(SEQ ID NO: 15)

5′ TGTGCGTCACAGCCAAAAGCCAGA 3′
EcdsdA-860F′

(SEQ ID NO: 16)

5′ TCGCATTCGGGCTTAAACTG 3′
EcdsdA-922R

(SEQ ID NO: 17)

5′ GCGTTGGTTCGGCAAAAA 3′
EcdsdA-883FAM

(SEQ ID NO: 18)

5′ TTTGGCGATCATGTTCACTGC 3′
dao1/pa-285F

(SEQ ID NO: 19)

5′GTT CGC GCA GAA CGA AGA C -3′
dao1/pa-349R

(SEQ ID NO: 20)

5′GGC GGT AAT TTG GCG TGA -3′
dao1/pa-308FAM

(SEQ ID NO: 21)

5′TCC TTG TAC CAG TGC CCG AGC A -3′
PCR Primers
For gusINT gene
Forward

(SEQ ID NO: 22)

5′-ACC GTT TGTGTGAACAACGA -3′
Reverse

(SEQ ID NO: 23)

5′-GGCACAGCACATCAAAGAGA -3′
For dsdA gene
Forward

(SEQ ID NO: 24)

5′-GCTTTTTGTTCGCTTGGTTGTG -3′,
Reverse

(SEQ ID NO: 25)

5′-TCAATAATCCCCCCAGTGGC- 3′
For dao1 gene
Forward

(SEQ ID NO: 26)

5′-GACAAGCAAAATGGGAAGAATC -3′,
Reverse

(SEQ ID NO: 27)

5′-TCGGGGAATGATGTAGGC -3′
For PAT gene
Forward

(SEQ ID NO: 28)

5′- ATGTCTCCGGAGAGGAGACCAGTTGAGAT-3′
Reverse

(SEQ ID NO: 29)

5′- GCCAAAAACCAACATCATGCCATCCA-3′

### Example 1

### Sensitivity of Barley Tissues on Elevated Concentrations of D-Serine and D-Alanine

### Germination of Immature Embryos

In order to establish effective concentrations of D-Serine and D-Alanine on inhibiting growth of tissue cultured barley cells, a bioassay system using immature embryos was applied. Immature embryos from spring barley variety Golden Promise 2 mm in length were dissected onto germination PAW-5 hormone free medium with D-serine or D-alanine in range of concentrations between 0 and 5 mM and maintained at 25° C. with a 16 h photoperiod. The number of germinated embryos with well-developed shoots and brunched roots were scored after 14 days.

Most of the embryos germinated while further seedlings growth was inhibited when roots emerged. Seedlings derived from embryos isolated from the immature caryopsis without endosperms were susceptible to the selection in concentration higher than 2 mM D-serine and 1 mM D-alanine (Table 5).

TABLE 5
In vitro germination of immature embryos on
medium containing D-serine and D-alanine.

| | |
|---|---|
| D-serine and D-alanine | Immature Embryos (%) |

| Concentrations (mM) | D-serine | D-alanine |
|---|---|---|
| 0 | 100 | 100 |
| 0.5 | 49 | 37 |
| 1 | 17 | 0 |
| 2 | 0 | 0 |
| 5 | 0 | 0 |

The uptake of the selection compounds via scutellum and later on with roots enable fast accumulation of the selection agents in the tissues and cause the lethal effect on immature embryos denomination within one week. Both compounds show to be lethal in the bioassay in concentrations 1 mM D-alanine and 2 mM D-serine.

### Example 2

### Regeneration of Transgenic Barley Plants Using dsdA Gene, PAW Set of the Medium and Selection on D-Serine

### 2.1 Preparation of Tissues for Transformation

### Plant Material

Donor plants were produced from spring barley Golden Promise in an environmental controlled growth chambers with a 16/8-h photoperiod at 300 μmol m⁻² s⁻¹ intensity and 70% humidity. The day night temperature was 20/16° C. Two well developed seedlings per 4.2 l square pots (8:1:1 Soil (K-jord): perlite: clay) (Weibulls, Sweden) were watered daily and fertilized 4 times during the vegetation including the basic fertilization with Superba vit (38 mg N per pot) (Weibulls, Sweden). Immature seeds were harvested 14 days after anthesis. Seeds from middle part of the spikes were collected for isolation of immature embryos.

### Seed Sterilization and Immature Embryos Isolation

Immature seeds were sterilized by washing in 96% EtOH for 30 seconds followed by steering in 10% commercial bleach (Klorin®)+0.1% Tween-20 on the shaker for 12 min and five times rinsing in sterile distilled water. Immature embryos were excised and bisected longitudinally through the root and shoot meristems aseptically under the stereomicroscope and collected in 1 ml PAW-infection medium with 300 μM acetoseringone added. Approximately 50 explants were collected per micro tube with an optimal size 1.5-2.0 mm in length, well-developed milky scutellum.

### 2.2 Constructs

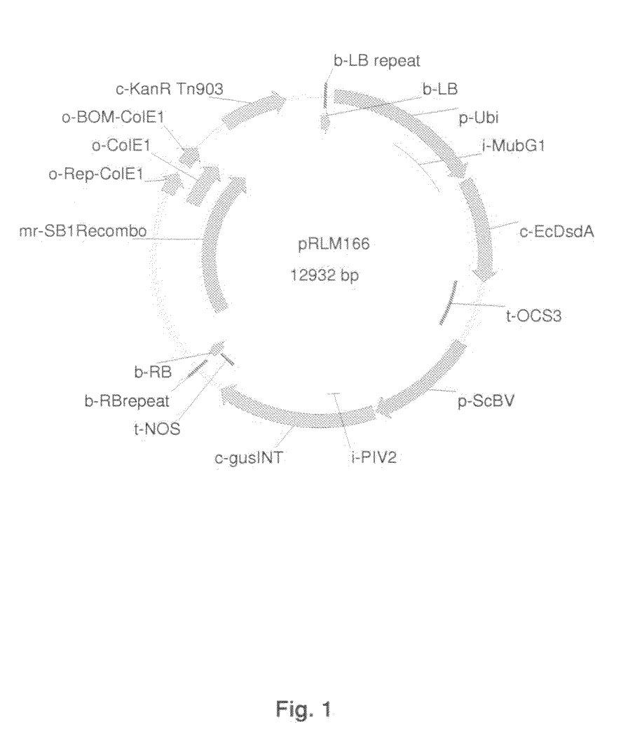

Super binary system was used in transformation experiments (WO94/00977 Japan Tobacco Inc). Cloning vector pSB 11 was modified by replacing Sp gene with Km gene that is resulting in intermediate cloning vector pRLM175. Expression cassettes with dsdA and gisINT genes were cloned between RB and LB of T-DNA in intermediate cloning vector pRLM175. Construct map of pRLM166 is shown in FIG. 1.

Integration into Agrobacterium Strain Carrying Super Binary Vector

The resulting intermediate plasmids were introduced by tri-parental mating cross (Ditta et al. 1980) Tri-parental mating is a term known in the art and involves a bacteria mating with 3 “sexes”.) in host bacteria LBA 4404 (pSB1) that has a helper plasmid pAL4404 (having a complete vir region) and super virulence plasmid pSB1 obtained by inserting virB, virc and virG genes of a strongly virulent Agrobacterium tumefaciens strain A281 into pRK2 replicon. Both super virulence and intermediate plasmids share the regions of homology and recombine in Agrobacterium. The presence of the transgenes in resulting recombined super binary vector system were confirmed in Agrobacterium by PCR using specific primers for dsdA (SEQ ID NO: 24, SEQ ID NO: 25) and gusINT (SEQ ID NO: 22, SEQ ID NO: 23).

### Preparation of Agrobacterium Inoculum for Transformation

Bacterial culture is initiated from the glycerol stock from the single colony growth on AB (Chilton et al. 1974) medium containing 50 mg/l kanamycin and 60 mg/l rifamlicin respectively. Plates were incubated at 28° C. in the dark for 3 days or until single colonies are visible. For transformation fresh Agrobacterium culture is initiated from single colony on agar plate with YEP medium containing 10 g/l peptone, 5 g/l yeast extract, 5 g/l NaCl 15 g/l OXOID agar, 50 mg/l kanamycin. Bacterial culture was grown for 2-3 days in dark at 26° C. Inoculum was initiated by dispersing Agrobacterium cells (5 loops 2 mm in 5 ml medium) into PAWInf. medium supplemented with 300 μM acetoseringone inverting and vortexing the tube for 5 min. Bacterial suspension was placed at 21° C. for 3 h on the shaker 200 rpm in dark. The density of cell population was adjusted to 1.0-1.2 O.D. measured at A 660 in spectrophotometer just before infection.

### 2.3 Transformation

Inoculation with Agrobacterium and Co-Cultivation

Explants were washed with PAWInf. medium and immersed in the above-described bacterial suspension for 2 h at 26° C. At the end of infection the explants were placed with scutellum side up on PAW-1 medium. Excesses bacterial suspension is removed by pipetting out and air-drying of the infected embryos by opening plates for 15 min on the sterile bench. Plates were sealed with Parafilm and placed in thermostat at 26° C. in the dark. Co-cultivation took place 5-6 days.

### Selection of Transgenic Callus and Tissues

After co-cultivation period the explants were washed with sterile water and 500 mg/L Cefotaxime and filter paper dried before being transferred to PAW-2 callus induction-recovery medium containing 160 mg/l Timentin for 14 days (7 days dark/7 days semi light; 13.2 μmol m⁻² s⁻¹). Explants with embryogenic callus were subculture to PAW-2 callus-proliferation medium containing 160 mg/l Timentin and corresponding selection 5 mM D-Serine. The selection on D-Amino acids was starting on PAW-2 callus induction medium 14 days after co cultivation. Embryogenic callus was subculture twice on fresh selective medium for callus proliferation and maintaining. Embryogenic callus regenerated on PAW-4 medium with 5 mM D-serine. Cultures were maintained at 23° C. on light 60.2 μmol m⁻² s⁻¹. Regenerated shoots were subculture to PAW-5 hormone free medium containing (5 mM D-Serine) for further growth and rooting. All media used in the transformation experiments were filter sterilized and are listed in General methods above. After analyses transgenic plants were transferred to soil and placed for further growth in greenhouse.

### 2.4 Transgene Inheritance

T1 progenies from each 5 T0 events with dsdA gene were analyzed for inheritance of the transgene. Transgenic nature of the progenies was confirmed by TaqMan real time PCR. The expression of dsdA in T1 seedlings was evaluated by germination test on selection medium containing 2 mM D-serine.

### 2.5 Results

Freshly isolated immature embryos from Golden Promise were inoculated with Agrobacterium suspension. Transformation experiments were conducted with pRLM166 (SEQ ID NO:10) construct carrying dsdA selectable marker gene (FIG. 1). Following co cultivation the explants were given a chance to recover for 14 days on callus induction selection free medium containing 160 mg/l Timentin to inhibit bacterial growth. Under these conditions 67% of the embryogenic callus developed over the scutellum. Embryogenic callus was transferred to the selection medium containing 5-mM D-serine. Transgenic callus lines tolerant to D-Serine were selected within 8 weeks with a frequency 1.2 to 11.3% (FIG. 4A). Vigorously grown transgenic callus lines were proved to be positive when tested for GUS expression using histochemical staining (Jefferson 1987 with additionally added 20% methanol) (FIG. 4 B). About 50% of the transgenic calluses regenerated with individual green transgenic plants. Plants were rooted and gown under constant selection pressure (5 mM D-Serine). Measured as production of transgenic lines transformation efficiencies was 2.1%-2.2% (Table 6.). Escape rate appeared in range 4.6%-13.8%.

TABLE 6
The selection of transgenic plants containing dsdA selectable
marker gene using Agrobacterium mediated transformation,
construct pRLM166 and selection on D-Serine

| Experiments | Explants | Selected callus | Transgenic | |
|---|---|---|---|---|
| No. | No. | lines | Plants | TE %* |
| 1 | 134 | 6 | 3 | 2.2 |
| 2 | 46 | 12 | 1 | 2.1 |

*TE-Transformation Efficiency calculated as % of transgenic plants out of the explants (freshly isolated immature embryos).

### Example 3

### Regeneration of Transgenic Barley Plants Using dsdA and dao1 Genes, PAB Set of the Medium and Selection on D-Serine

### 3.1 Preparation of Tissues for Transformation

### Plant Material

Donor plants were produced as it was described in Example 2.

### Seed Sterilization and Immature Embryos Isolation

Seeds were sterilized and isolated as it was described above. Immature embryos were excised and bisected longitudinally through the root and shoot meristems aseptically under the stereomicroscope and placed directly on the surface of PAB-1 medium with 300 μM acetosyringone added. Approximately 50 explants per plate were collected with an optimal size 1.5-2.0 mm in length.

### 3.2 Constructs

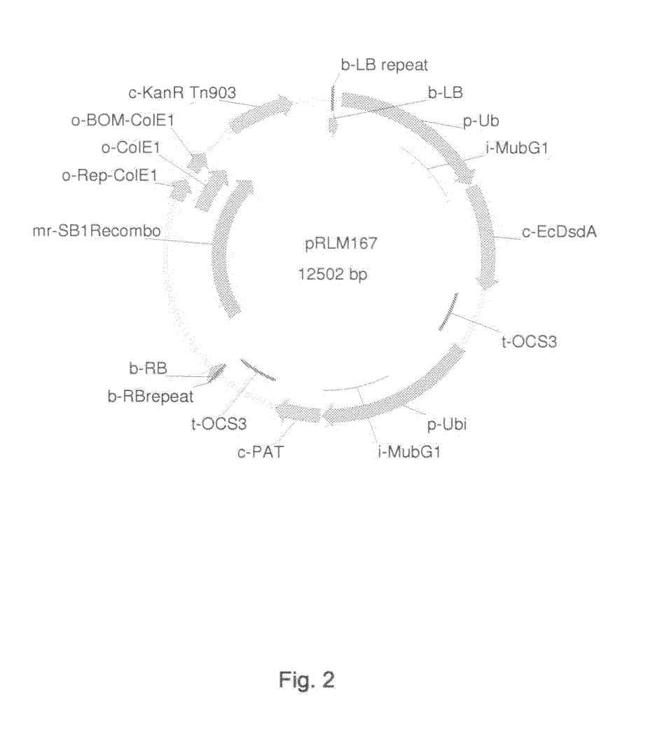

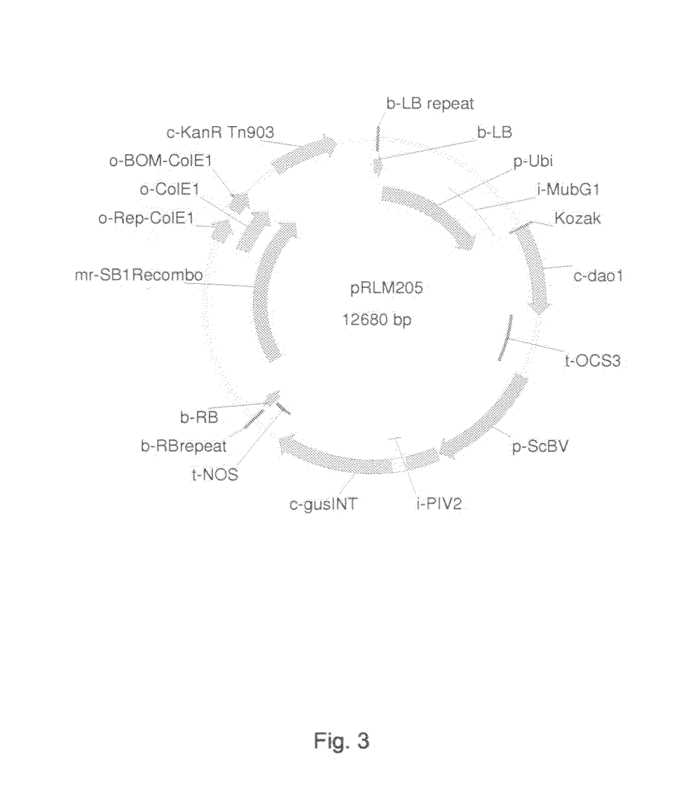

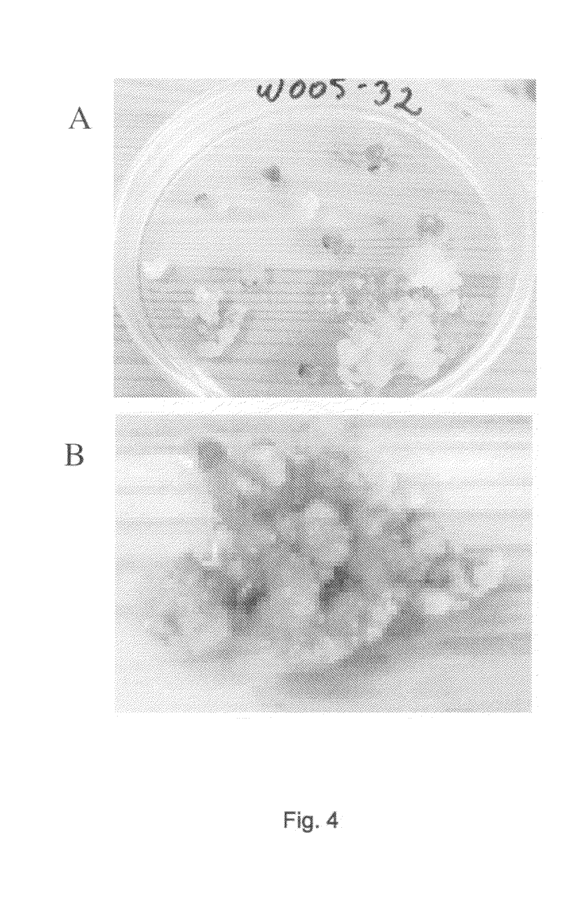

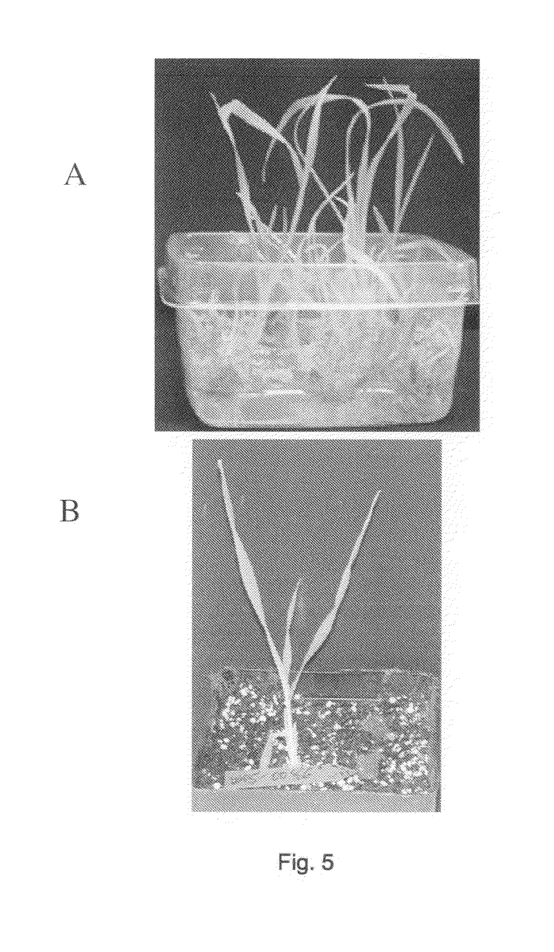

Super binary system was used in transformation experiments (WO94/00977 Japan Tobacco Inc). Cloning vector pSB 11 was modified by replacing Sp gene with Km gene that is resulting in intermediate cloning vector pRLM175. Expression cassettes with dsdA, dao1, gusINT and PAT genes were cloned between RB and LB of T-DNA in intermediate cloning vector pRLM175. Construct maps of pRLM167 and pRLM205 are shown in FIG. 2 and FIG. 3.

Integration into Agrobacterium Strain Carrying Super Binary Vector

The resulting intermediate plasmids were introduced by tri-parental mating cross (Ditta et al. 1980) Tri-parental mating is a term known in the art and involves a bacteria mating with 3 “sexes”.) in host bacteria LBA 4404 (pSB1) that has a helper plasmid pAL4404 (having a complete vir region) and super virulence plasmid pSB1 obtained by inserting virB, virC and virG genes of a strongly virulent Agrobacterium tumefaciens strain A281 into pRK2 replicon. Both super virulence and intermediate plasmids share the regions of homology and recombine in Agrobacterium. The presence of the transgenes in resulting recombined super binary vector system were confirmed in Agrobacteria by PCR using specific primers for: dsdA (SEQ ID NO: 24, SEQ ID NO: 25), PAT (SEQ ID NO: 28, SEQ ID NO: 29), gusINT (SEQ ID NO: 22, SEQ ID NO: 23), dao1 (SEQ ID NO: 26, SEQ ID NO: 27).

### Preparation of Agrobacterium Inoculum for Transformation

Bacterial culture is prepared as it is described in Example 2 with an exception that the bacteria is dispersed in PABInf. medium supplemented with 300 μM acetosyringone.

### 3.3 Transformation

Inoculation with Agrobacterium and Co-Cultivation

Explants were inoculated by dripping the 20 μl bacterial suspension on the explants surface. Infection took place on the sterile bench for 2 h at room temperature. Excesses bacterial suspension was removed with filter paper. Plates were sealed with Parafilm and placed in thermostat at 24° C. in the dark. Co-cultivation took place 4-5 days.

### Selection of Transgenic Callus and Tissues

After co-cultivation period the explants were washed with sterile water and 500 mg/L Cefotaxime and filter paper dried before being transferred to PAB-2 callus induction-recovery medium containing 160 mg/l Timentin for 7 days in dark. Explants with embryogenic callus were subculture to PAB-3 callus-proliferation medium containing 160 mg/l Timentin and corresponding selection: 5 mM D-Serine when transformed with pRLM205 and pRLM167 or 5 mg/l bialaphos when transformed with double selectable markers construct pRLM167. Embryogenic callus was subculture twice on fresh selective medium for callus proliferation. Green plants were regenerated on PAB-4 medium with corresponding selection 3 mM D-Serine and 1 mg/l bialaphos. Cultures were maintained at 23° C. on light 60.2 μmol m⁻² s⁻¹. Regenerated shoots were subculture to PAB-5 hormone free medium with corresponding selection (5 mM D-Serine or 3 mg/l bialaphos) for further growth and rooting. All media used in the transformation experiments were filter sterilized and are listed in General methods above. After analyses transgenic plants were transferred to soil and placed for further growth in greenhouse.

### 3.4 Results

Freshly isolated immature embryos from Golden Promise were inoculated with Agrobacterium suspension by dripping on the explants surface. Transformation experiments were conducted with both pRLM167 (SEQ ID NO 11) and pRLM 205 (SEQ ID N012). Following co cultivation the explants were given a chance to recover for 7 days on callus induction selection free medium containing 160 mg/l Timentin to inhibit bacterial growth. Under these conditions 79% of the embryogenic callus developed over the scutellum. Embryogenic callus was transferred to the selection medium containing 5-mM D-serine or in case of pRLM167 5 mg/l bialaphos was used. Transgenic callus lines tolerant to D-Serine and bialaphos were selected within 8 weeks with a frequency 1.2-11.3%. Vigorously grown transgenic callus lines were transferred for regeneration and about 25-50% out of them regenerated on PAB-4 medium with green transgenic plants. Plants were rooted and gown under constant selection pressure (5 mM D-Serine) (FIG. 5A). Plants were acclimatized and transferred to the greenhouse for further growth and development (FIG. 5B). Transgenic plants were selected on both D-Serine and bialaphos using double construct pRLM 167. Measured as production of transgenic lines transformation efficiencies were 4.6% when pRLM167 was used and transgenic plants were selected on medium on D-Serine while the selection on bialaphos resulted with 3.4 TE % (Table 7). Escape rate was in frequency 3.5% to 8.6% when D-Serine was used and 4.6%- to 13.8% when bialaphos was applied. Transgenic plants were also selected on D-Serine when pRLM 205 carrying dao1 gene was used in transformation resulted with transformation efficiency 3.6% (Table 7).

TABLE 7
The selection of transgenic plants containing dsdA and dao1 selectable
marker genes using Agrobacterium mediated transformation approach
and selection on D-Serine and bialaphos

| Experiments | | | Explants | Selected | Transgenic | |
|---|---|---|---|---|---|---|
| No. | Constructs | Selection | No. | callus lines | Plants | TE %* |
| 1 | pRLM167 | D-serine | 65 | 5 | 3 | 4.6 |
| 2 | pRLM167 | Bialaphos | 58 | 8 | 2 | 3.4 |
| 3 | PRLM205 | D-serine | 55 | 4 | 2 | 3.6 |

*TE—Transformation Efficiency calculated as % of transgenic plants out of the ex-plants (freshly isolated immature embryos).

The experiments suggest that the D-amino acid selection system is resulting with transgenic barley plants in higher transformation efficiency compared to selection on bialaphos. Additional advantage is that the escape rate was lower when D-amino acid selection was used. Both evaluated protocols with PAW set and PAB set of medium were resulting with transgenic plants. Regeneration and transformation performance of barley callus was significantly improved when PAB set of media was tested. Both genes dasA and dao1 were successfully introduced and expressed in barley tissues.

### REFERENCES

The references listed below and all references cited herein are incorporated herein by reference to the extent that they supplement, explain, provide a background for, or teach methodology, techniques, and/or compositions employed herein.
1. An et al. (1985) EMBO J 4:277-287
2. Anderson & Gregeson (1989) Genome 31:994-999
3. Anderson and Young, Quantitative Filter Hybridization, in Nucleic Acid Hybridization (1985)
4. Ashby et al. (1988) J. Bacteriol. 170: 4181-4187
5. Atanassova et al. (1992) Plant J 2(3): 291-300
6. Ausubel F M et al. (1987) Current Protocols in Molecular Biology, Greene Publishing Assoc. and Wiley Interscience
7. Baker et al. (1987) EMBO J 6: 1547-1554
8. Ball. J. B. and Alewood, P. F. (1990) J. Mol. Recognition 3:55
9. Barnett T. et al. (1980) Dev. Genet. 1:331-340
10. Barry et al. (1992) p. 139-145 in: B. K. Singh et al. (ed.) Biosynthesys and Molecular Regulation of Amino Acids in Plants. Am. Soc. Plant Physiologists Rockville, Md.
11. Bernnasconi P et al. (1995) J. Biochem. Chem. 29:17381-17385
12. Bevan et al. (1984) Nucl Acid Res 12, 8711-8720
13. Binding, Regeneration of Plants, Plant Protoplasts, pp. 21-73, CRC Press, Boca Raton (1985)
14. Binet et al. (1991) Plant Science 79:87-94
15. Bolton et al. (1986) Science 232: 983-985;
16. Breathnach R. and P. Chambon (1981) Ann. Rev. Biochem. 50:349-383
17. Brinch_Petersen (1996) Plant Mol. Biol. 32: 611-620
18. Brinch_Petersen et al. (1999) Hereditas 131: 239-245
19. Broothaerts W et al. (2005) Nature 433:629-633
20. Callis et al. (1990) J Biol Chem 265(21):12486-12493
21. Callis et al., “Ubiquitin and Ubiquitin Genes in Higher Plants,” Oxford Surveys of Plant Molecular & Cell Biology, vol. 6, pp. 1-30 (1989)
22. Callis et al., J. Biol. Chem. 265(21):12486-12493 (1990)
23. Chen and Winans (1991) J. Bacteriol. 173: 1139-1144
24. Chilton et al. (1974) Proc. Natl. Acad. Sci. USA 71, 3672-6
25. Christensen et al. (1992) Plant Mol Biol, 18:675-689
26. Christensen et al. (1996) Transgenic Res 5:213-218
27. Christou et al. (1988) Plant Physiol 87:671-674
28. Christou et al. (1991) Biotechnology 9: 957-962
29. Crameri et al. (1997) Nature Biotech. 15:436
30. Crameri et al., Nature, 391:288 (1998)

31. Currie et al. (1995) Weed Sci. 43:578-582
32. Cushman et al. (2000) Curr Opin Plant Biol 3(2):117-24
33. Dale & Ow (1991) Proc Nat'l Acad Sci USA 88:10558-10562
34. Dandekar et al. (1989) J Tissue Cult Meth 12:145
35. de Block et al. (1987) EMBO J 6:2513-2518
36. de Bruijn et al. (1996) Rep-PCR Genomic Fingerprinting of Plant-Associated Bacteria and Computer-Assisted Phylogenetic Analyses In: Biology of Plant-Microbe Interaction; Proceedings of the 8th International Congress of Molecular Plant-Microbe Interactions (G. Stacey, B. Mullin and P. Gresshoff, Eds.) APS Press, 497-502
37. De la Pena et al. (1987) Nature 325:274-276
38. Deblaere et al. (1985) Nucl Acids Res 13:4777-4788
39. Dekeyser et al. (1989) Plant Physiol. 90: 217-223
40. Della-Cioppa et al. (1987) Plant Physiology 84:965-968
41. Della-Cioppa et al. BioTechnology 5:579-584 (1987)
42. Ditta et al. (1980) Proc. Natl. Acad. Sci. USA 77: 747-751
43. Dixon M & Kleppe Biochim. Biophys. Acta 96 (1965c) 383-389
44. Dixon M & Kleppe K Biochim. Biophys. Acta 96 (1965b) 368-382
45. Dixon M & Kleppe K. Biochim. Biophys. Acta 96 (1965a) 357-367
46. Du et al. (1989) Genet Manip Plants 5:8-12
47. Dunwell J M (2000) J Exp Bot 51 Spec No: 487-96
48. Eichholtz et al. (1987) Somatic Cell and Molecular Genetics 13: 67-76
49. Elsen et al. (1985) Plant Mol. Biol. 5: 299-305
50. EP-A 120 516
51. EP-A 175 966
52. EP-A 270,356
53. EP-A 290 395
54. EP-A 331 083
55. EP-A 333 033
56. EP-A 434 616
57. EP-A 444 882
58. EP-A 672 752
59. EP-A 709 462
60. Erikson et al. (2004) Nature Biotechnology 22: 455-458
61. Erikson et al. (2004) Plant Molecular Biology vol. 53: (3) 425-433
62. Evans et al., Protoplasts Isolation and Culture, Handbook of Plant Cell Culture, pp. 124176, Macmillian Publishing Company, New York (1983)
63. Farmer, P. S. in Drug Design (E. J. Ariens, ed.) Academic Press, New York, 1980, vol. 10, pp. 119-143
64. Farrand et al. (2003) Int. J. Systematic & Evolutionary Microbiology 53:1681-1687
65. Fedoroff N V & Smith D L (1993) Plant J 3:273-289
66. Fire A. et al (1998) Nature 391:806-811
67. Fraley et al. Proc Natl Acad Sci USA 80: 4803 (1983)
68. Frame et al. (2002) Plant Physiol. 129: 13-22
69. Freeman et al. (1984) Plant Cell Physiol 2 9:1353
70. Fromm et al. (1985) Proc Natl Acad Sci USA 82:5824
71. Fromm et al. (1990) Bio/Technology 8:833-839
72. Funatsuki et al. (1995) TAG 91: 707-712
73. Gabler M et al. (2000) Enzyme Microb. Techno. 27, 605-611
74. Gallie et al. (1987) Nucl Acids Res 15:8693-8711
75. Garbarino et al. (1992) Plant Mol Biol 20:235-244
76. Gelvin et al. (eds) (1990) Plant Molecular Biology Manual; Kluwer Academic Publisher, Dordrecht, The Netherlands
77. Genschick et al. (1994) Gene, 148:195-202
78. Goeddel; Gene Expression Technology: Methods in Enzymology 185, Academic Press, San Diego, Calif. (1990)
79. Green et al. (1987) Plant Tissue and Cell Culture, Academic Press
80. Gruber et al. (1993) “Vectors for Plant Transformation,” in METHODS IN PLANT MOLECULAR BIOLOGY AND BIOTECHNOLOGY; CRC Press, Boca Raton, Fla., eds.: Glick and Thompson, Chapter 7, pp. 89-119.
81. Guivarc'h et al. (1993) Protoplasma 174:10-18
82. Hagio et al. (1995) Plant Cell Report 14: 329-334
83. Hajdukiewicz et al. (1994) Plant Mol Biol 25:989-994
84. Hansen et al. (1994) Proc. Natl. Acad. Sci. USA 91:7603-7607
85. Hiei et al. (1994) Plant J 6: 271-282
86. Higo et al. (1999) Nucl Acids Res 27(1): 297-300
87. Hirschman, R., et al. (1993) J. Am. Chem. Soc. 115:12550-12568
88. Hoekema (1985) In: The Binary Plant Vector System, Offsetdrukkerij Kanters B.V., Alblasserdam, Chapter V
89. Hoekema et al. (1983) Nature 303:179-181
90. Hoffman et al. (1991) Mol Biol 17:1189-1201
91. Holsters et al. (1978) Mol Gen Genet 163:181-187
92. Holtorf et al. (1995) Plant Mol Biol 29: 637-747
93. Hood E E, Jilka J M. (1999) Curr Opin Biotechnol. 10(4):382-386
94. Hood et al. (1986) J Bacteriol 168:1291-1301
95. Hooykaas et al. (1977) J Gen Microbiol 98:477-484
96. Ishida et al. (1996) Nature Biotech 745-750
97. Jacq et al. (1993) Plant Cell Reports 12: 621-624
98. Jähne et al. (1994) TAG 989: 525-533
99. James et al. (1993) Plant Cell Reports 12: 559-563
100. Jarchow et al. (1991), Proc. Natl. Acad. Sci. USA 88:10426-10430
101. Jefferson et al. (1987) EMBO J. 6: 3901-3907
102. Jensen et al. (1996) Proc. Nat. Acad. Sci. USA 93: 3487-34-91
103. Jones, H. D. (2005) J. of Cereal Science 41: 137-147
104. Kado (1991) Crit Rev Plant Sci 10:1
105. Kawalleck et al. (1993) Mol Biol 21:673-684
106. Kemper et al. (1992) Plant Cell Rep 11: 118-121
107. Keown et al. (1990) Meth Enzymol 185:527-537
108. Khana, H. K. & Daggar, G. E. (2003) Plant Cell Rep 21: 429-436
109. Kihara et al. (1998) 17: 937-940
110. Kilby N J et al. (1995) Plant J 8:637-652
111. Kishore et al. (1992) Weed Technol. 6: 626-634
112. Klapwijk et al. (1980) J. Bacteriol., 141, 128-136
113. Klee et al. (1987) Ann Rev Plant Physiol 38:467-486.
114. Klein & Klein (1953) J Bacteriol. 66 (2): 220-228;
115. Klein et al. (1987) Nature 327:70-73
116. Koncz & Schell (1986) Mol Gen Genet 204:383-396
117. Koprek et al. (1996) Plant Science 119: 79-91
118. Kramer at al. (1993) Planta 190: 454-458
119. Lawson et al. (1994) Mol Gen Genet 245:608-615
120. Lazzeri et al. (1991) TAG 81: 437-444
121. Lazzeri et al. (1997) Aspects of Applied Biology 50:1-8
122. Lazzeri P (1995) Methods Mol Biol 49:95-106
123. Lemaux et al. In I. Vasil (ed) Molecular Improvement of Cereal Crop, Kluwer Academ Publisher, The Netherland
124. Lepetit et al. (1992) Mol. Gen. Genet. 231: 276-285
125. Lescot et al. Nucleic Acids Res 30(1):325-7 (2002)
126. Li et al. (1992) Plant Mol Biol 20:1037-1048
127. Liu L et al. (1995) Biochem Cell Biol. 73(1-2):19-30
128. Llob et al. (2003) Europ J Plant Pathol 109:381-389
129. Luo and Wu (1988) Plant Mol. Biol. Rep. 6:165-174
130. Lysnik et al. (1993) NAR 21:969-975
131. Lyznik et al. (1996) Nucleic Acids Res 24:3784-3789
132. Ma J K and Vine N D (1999) Curr Top Microbiol Immunol, 236:275-92
133. Maniatis T, Fritsch E F and Sambrook J (1989) Molecular Cloning: A Laboratory Manual, 2nd Ed., Cold Spring Harbor Laboratory, Cold Spring Harbor (NY)
134. Massey V et al. Biochim. Biophys. Acta 48 (1961) 1-9
135. Matzke M A et al. (2000) Plant Mol Biol 43:401-415
136. McElroy et al., Plant Cell 2: 163171 (1990)
137. McGranahan et al. (1990) Plant Cell Rep 8:512
138. Meister A & Wellner D Flavoprotein amino acid oxidase. In: Boyer, P. D., Lardy, H. and Myrbäck, K. (Eds.), The Enzymes, 2nd ed., vol. 7, Academic Press, New York, 1963, p. 609-648
139. Melchers et al. (2000) Curr Opin Plant Biol 3(2):147-52
140. Messing J. et al. (1983), in Genetic Engineering of Plants, T. Kosuge, C. Meredith and A. Hollaender (eds.), Plenum Press, pp. 211-227
141. Miki et al. (1993) “Procedures for Introducing Foreign DNA into Plants” in METHODS IN PLANT MOLECULAR BIOLOGY AND BIOTECHNOLOGY; pp. 67-88
142. Miyano M et al. (1991) J Biochem 109:171-177
143. Mol J N et al. (1990) FEBS Lett 268(2):427-430
144. Moloney et al. (1989) Plant Cell Reports 8: 238
145. Montell C. et al. (1983) Nature 305:600-605
146. Moore et al. (1997) J. Mol. Biol., 272:336
147. Morgan, B. A. and Gainor, J. A. (1989) Ann. Rep. Med. Chem. 24:243; and Freidinger, R. M. (1989) Trends Pharmacol. Sci. 10:270
148. Mozo & Hooykaas (1991) Plant Mol. Biol. 16:917-918
149. Murashige, T. & Skoog, F. (1962) Physiologia Pl. 15: 473-497
150. Nehra et al. (1994) Plant J. 5:285-297
151. Newhouse et al. (1991) Theor Appl Gene. 83:65-70
152. Odell et al. (1990) Mol Gen Genet 223:369-378
153. Olhoft et al. (2001) Plant Cell Rep 20: 706-711
154. Onouchi et al. (1995) Mol Gen Genet 247:653-660
155. Osborne et al. (1995) Plant J. 7, 687-701
156. Ow et al. (1986) Science 234:856-859
157. Paszkowski et al. (1984) EMBO J 3:2717-2722
158. Patel et al. (2000) Mol Breed 6: 113-123
159. Pelham and Bienz (1982) EMBO J. 1:1473-1477
160. Perl et al. (1996) Nature Biotechnol 14: 624-628
161. Pilone, M. S. (2000) Cell. Mol. Life Sci. 57:1732-1742
162. Potrykus (1990) Bio/technology. 8, 535-542.
163. Potrykus (1991) Ann Rev Plant Physiol Plant Mol Biol 42:205-225
164. Ritalla et al. (1994) Plant Cell Report 24: 317-325
165. Rouster J et al. (1998) Plant J 15:435-440
166. Russell et al. (1992) Mol Gene Genet 234: 49-59
167. Saijo et al. (2000) Plant J 23(3): 319-327
168. Sakamoto et al. (2000) J Exp Bot 51(342):81-8
169. Sanford J C (1990) Physiologia Plantarium 79:206-209
170. Sauer B (1998) Methods 14(4):381-92
171. Sautter et al. (1991) Bio/Technology, 9:1080-1085
172. Sawada et al. (1993) International Journal of Systematic Bacteriology 43(4):694-702
173. Sawyer, T. K. (1995) “Peptidomimetic Design and Chemical Approaches to Peptide Metabolism” in Taylor, M. D. and Amidon, G. L. (eds.) Peptide-Based Drug Design Controlling Transport and Metabolism, Chapter 17
174. Scheeren-Groot et al. (1994) J. Bacteriol 176: 6418-6426
175. Shah et al. (1986) Science 233: 478
176. Sheehy et al. (1988) Proc Natl Acad Sci USA 85: 8805-8809
177. Sheen et al. (1995) Plant J 8(5):777-784
178. Shewmaker et al. (1985) Virology 140:281-288
179. Shillito et al. (1985) Bio/Technology, 3:1099-1103
180. Shimamoto et al. (1992) Nature 338:274-276
181. Shimamoto K (1994) Current Opinion in Biotechnology 5:158-162
182. Shirsat A et al. (1989) Mol Gen Genet 215(2): 326-331
183. Silhavy T J, Berman M L and Enquist L W (1984) Experiments with Gene Fusions, Cold Spring Harbor Laboratory, Cold Spring Harbor (N.Y.)
184. Smith, A. B. 3rd, et al. (1994) J. Am. Chem. Soc. 116:9947-9962
185. Smith, A. B. 3rd, et al. (1995) J. Am. Chem. Soc. 117:11113-11123
186. Stachel et al. (1985) Nature 318: 624-629
187. Stemmer (1994a) Nature, 370:389-391
188. Stemmer (1994b) Proc Natl Acad. Sci USA 91:10747-10751
189. Stryer, Biochemistry (1988) W. H. Freeman and Company, New YorkH
190. Sugita K et al. (2000) Plant J. 22:461-469
191. Suzuki (2001) Gene. January 24; 263(1-2):49-58
192. The Maize Handbook, Chapter 116, Freeling and Walbot, Eds., Springer, New York (1994)
193. Thomson et al. (1987) Plant Physiolo 104: 2519-2523
194. Tingay et al. (1997) Plant J. 11: 1369-1376
195. Travella et al. (2005) Plant Cell Report 23: 780-789
196. Trifonova et al. (2001) Plant Science 161: 870-880
197. U.S. Pat. No. 4,761,373
198. U.S. Pat. No. 4,801,340
199. U.S. Pat. No. 4,940,838
200. U.S. Pat. No. 4,975,374
201. U.S. Pat. No. 5,100,792
202. U.S. Pat. No. 5,225,341
203. U.S. Pat. No. 5,304,732
204. U.S. Pat. No. 5,605,793
205. U.S. Pat. No. 5,750,866
206. U.S. Pat. No. 5,811,238
207. U.S. Pat. No. 5,830,721
208. U.S. Pat. No. 5,837,458
209. U.S. Pat. No. 6,100,447
210. U.S. Pat. No. 6,528,701
211. U.S. Pat. No. 6,541,257
212. U.S. Pat. No. 6,653,529
213. Vain et al. (1995) Biotechnology Advances 13(4):653-671
214. Van Laerebeke et al. (1974) Nature 252, 169-170
215. van Veen R J M et al. (1988) Mol Plant Microb Interact 1(6):231-234
216. Van Wordragen and Dons (1992) Plant Mol. Biol. Rep. 10: 12-36
217. Vanden Elzen et al. (1985) Plant Mol Biol. 5:299
218. Vasil (1996) Nature Biotechnology 14:702
219. Vasil et al. (1992) Bio/Technology, 10:667-674
220. Vasil et al. (1993) Bio/Technology, 11:1153-1158
221. Vasil et al., Cell Culture and Somatic Cell Genetics of Plants, Vol I, II, and III, Laboratory Procedures and Their Applications, Academic Press, 1984,
222. Vernade et al. (1988) J. Bacteriol. 170: 5822-5829
223. Vinuesa et al. (1998) Appl. Envir. Microbiol. 64:2096-2104
224. WO01/18220
225. Wader et al. 1987 Tomato Technology 189-198 Alan R. Liss, Inc.
226. Wan & Lemaux (1994) Plant Physiol. 104:3748
227. Waterhouse P M et al. (1998) Proc Natl Acad Sci USA 95:13959-64
228. Watson et al. (1975) J. Bacteriol 123, 255-264
229. Watson et al. (1985) EMBO J 4(2):277-284
230. Weeks et al. (1993) Plant Physiol 102:1077-1084
231. Weissbach and Weissbach, Methods for Plant Molecular Biology, Academic Press, 1989
232. Wingender E et al. Nucleic Acids Res 29(1):281-3 (2001)
233. WO0/58484
234. WO 02/00900
235. WO 02/10415
236. WO 03/004659
237. WO 03/060133
238. WO 03/102198
239. WO 87/06614
240. WO 91/02071
241. WO 91/13991
242. WO 92/09696
243. WO 93/18168
244. WO 93/24640
245. WO 94/00583
246. WO 94/00977
247. WO 95/06722
248. WO 95/15389
249. WO 97/037012
250. WO 99/16890
251. WO 00/44895
252. WO 00/44914
253. WO 00/49035
254. WO 00/63364
255. WO 00/68374
256. WO 99/32619
257. WO 99/53050
258. Yeo et al. (2000) Mol Cells 10(3):263-8
259. Young et al. (2003) Int. J. Systematic & Evolutionary Microbiology 51:89-103
260. Zhang et al. (1997) Proc. Natl. Acad. Sci. USA, 94:4504
261. Zubko et al. (2000) Nature Biotech 18(4):442-445
262. Zuou et al. (2002) Plant J. 30: 349-359
263. Zupan et al. (2000) Plant J 23(1):11-2

1. A method for generating a transgenic barley plant comprising the steps of
a. introducing into a barley cell or tissue a DNA construct comprising at least one first expression construct comprising a promoter active in said barley plant and operably linked thereto a nucleic acid sequence encoding an enzyme capable of metabolizing D-alanine and/or D-serine,
b. incubating said barley cell or tissue of step a) on a selection medium comprising D-alanine and/or D-serine and/or a derivative, comprising the D-amino acid employed
i. modified by an amino-terminal group,
1. wherein the amino-terminal modifying group is selected from the group consisting of phenylacetyl, diphenylacetyl, triphenylacetyl, butanoyl, isobutanoyl hexanoyl, propionyl, 3-hydroxybutanoyl, 4-hydroxybutanoyl, 3-hydroxypropionoyl, 2,4-dihydroxybutyroyl, 1-Adamantanecarbonyl, 4-methylvaleryl, 2-hydroxyphenylacetyl, 3-hydroxyphenylacetyl, 4-hydroxyphenylacetyl, 3,5-dihydroxy-2-naphthoyl, 3,7-dihydroxy-2-napthoyl, 2-hydroxycinnamoyl, 3-hydroxycinnamoyl, 4-hydroxycinnamoyl, hydrocinnamoyl, 4-formylcinnamoyl, 3-hydroxy-4-methoxycinnamoyl, 4-hydroxy-3-methoxycinnamoyl, 2-carboxycinnamoyl, 3,4-dihydroxyhydrocinnamoyl, 3,4-dihydroxycinnamoyl, trans-Cinnamoyl, (±)-mandelyl, (±)-mandelyl-(±)-mandelyl, glycolyl, 3-formylbenzoyl, 4-formylbenzoyl, 2-formylphenoxyacetyl, 8-formyl-1-napthoyl, 4-(hydroxymethyl)benzoyl, 3-hydroxybenzoyl, 4-hydroxybenzoyl, 5-hydantoinacetyl, L-hydroorotyl, 2,4-dihydroxybenzoyl, 3-benzoylpropanoyl, (±)-2,4-dihydroxy-3,3-dimethylbutanoyl, DL-3-(4-hydroxyphenyl)lactyl, 3-(2-hydroxyphenyl)propionyl, 4-(2-hydroxyphenyl)propionyl, D-3-phenyllactyl, 3-(4-hydroxyphenyl)propionyl, L-3-phenyllactyl, 3-pyridylacetyl, 4-pyridylacetyl, isonicotinoyl, 4-quinolinecarboxyl, 1-isoquinolinecarboxyl and 3-isoquinolinecarboxyl,
ii. or modified by a carboxy-terminal modifying group,
1. wherein the carboxy-terminal modifying group is selected from the group consisting of an amide group, an alkyl amide group, an aryl amide group and a hydroxy group,
iii. or by modification of the side-chain,
iv. or N-alkyl (or aryl) substitutions, or backbone crosslinking to construct lactams and other cyclic structures,
v. or C-terminal hydroxymethyl derivatives,
vi. or O-modified derivatives,
vii. or N-terminally modified derivatives including substituted amides,
viii. or D-amino acid structure comprising herbicidal compounds
1. wherein herbicidal compounds are selected from the group of N-benzoyl-N-(3-chloro-4-fluorophenyl)-DL-alanine, N-benzoyl-N-(3-chloro-4-fluorophenyl)-DL-alanine methyl ester, N-benzoyl-N-(3-chloro-4-fluorophenyl)-DL-alanine ethyl ester, N-benzoyl-N-(3-chloro-4-fluorophenyl)-D-alanine, N-benzoyl-N-(3-chloro-4-fluorophenyl)-D-alanine methyl ester, and N-benzoyl-N-(3-chloro-4-fluorophenyl)-D-alanine isopropyl ester,
thereof in a total concentration from 1 mM to about 15 mM of D-alanine and/or 3 to 10 mM of D-serine for a time period of at least 5 days, and
c. transferring said barley cell or tissue of step b) to a regeneration medium and regenerating and selecting barley plants comprising said DNA construct.

2. The method of claim 1, comprising
a. isolating an immature embryo of a barley plant, and
b. co-cultivating said isolated immature embryo, which has not been subjected to a dedifferentiation treatment, with a bacterium belonging to genus Rhizobiaceae comprising at least one transgenic T-DNA, said T-DNA comprising at least one first expression construct comprising a promoter active in said barley plant and operably linked thereto a nucleic acid sequence encoding an enzyme capable to metabolize D-alanine and/or D-serine, and
c. transferring the co-cultivated immature embryos to a recovering medium, said recovery medium lacking a phytotoxic effective amount of D-serine or D-alanine, and
d. inducing formation of embryogenic callus and selecting transgenic callus on a medium comprising,
i. an amount of at least one auxin compound effective to stimulate cellular elongation and division and induce dedifferentiation (callus formation), and
ii. D-alanine and/or D-serine in a total concentration from 1 mM to about 15 mM of D-alanine and/or 3 to 10 mM of D-serine, and
e. regenerating and selecting plants containing the transgenic T-DNA from the said transgenic callus.

3. The method of claim 1, wherein the DNA construct further comprises at least one second expression construct conferring to said barley plant an agronomically valuable trait.

4. The method of claim 2, wherein the effective amount of the auxin compound is equivalent to a concentration of 0.2 mg/l to 6 mg/l 2,4-D or to a concentration of 0.2 ng/l to 6 mg/l Dicamba.

5. The method of claim 1, wherein the enzyme capable of metabolizing D-alanine or D-serine is selected from the group consisting of D-serine ammonia-lyases (EC 4.3.1.18), D-Amino acid oxidases (EC 1.4.3.3), and D-Alanine transaminases (EC 2.6.1.21).

6. The method of claim 1, wherein the enzyme capable of metabolizing D-serine is the D-serine ammonia-lyase (EC 4.3.1.18) whose organism of origin is selected from the group of organisms consisting of Bacillus subtilis, Escherichia coli, Vibrio colera and Bacillus halodurans,
and wherein selection is done on a medium comprising D-serine in a concentration from 3 mM to 10 mM;
or wherein the enzyme capable of metabolizing D-serine and D-alanine is D-amino acid oxidase (EC 1.4.3.3) whose organism of origin is selected from the group consisting of
Caenorhabditis elegans, Cavia parcellus (Guinea pig), Cricetulus griseus, Drosophila melanogaster, Fusarium solani, Fusarium solani (subsp. pisi), Homo sapiens (Human), Mus musculus (Mouse), Mycobacterium leprae, Neisseria meningitides, Neurospora crassa, Oryctolagus cuniculus, Rattus norvegicus (Rat), Rhodosporidium toruloides, Schizosaccharomyces pombe (Fission yeast), Streptomyces avermitilis, Streptomyces coelicolor, Sus scrofa (Pig), Trigonopsis variabilis, Xanthomonas axonopodis and Xanthomonas campestris
and wherein selection is done on a medium comprising D-alanine and/or D-serine in a total concentration from 3 mM to 10 mM.

7. The method of claim 1, wherein the enzyme capable of metabolizing D-serine is selected from the group consisting of
i) the E. coli D-serine ammonia-lyase as encoded by SEQ ID NO: 2,
ii) an enzyme having the same enzymatic activity and an identity of at least 80% to the sequence as encoded by SEQ ID NO: 2, and
iii) an enzyme encoded by a nucleic acid sequence capable of hybridizing under stringent conditions to the complement of the sequence described by SEQ ID NO: 1, wherein stringent conditions are defined by
a. either a combination of conditions comprising
i. salt concentrations of less than about 1.5 M,
ii. pH 7.0 to 8.3 and
iii. a temperature of at least about 30° C.,
b. or addition of an effective amount of at least one DNA duplex destabilizing agent,
and wherein selection is done on a medium comprising D-serine in a concentration from 3 to 10 mM; or
wherein the enzyme capable of metabolizing D-serine and D-alanine is selected from the group consisting of
i) the Rhodotorula gracilis D-amino acid oxidase as encoded by SEQ ID NO: 4,
ii) an enzyme having the same enzymatic activity and an identity of at least 80% to the sequence as encoded by SEQ ID NO: 4, and
iii) an enzyme encoded by a nucleic acid sequence capable of hybridizing to the complement of the sequence described by SEQ ID NO: 3,
and wherein selection is done on a medium comprising D-alanine and/or D-serine in a total concentration from 1 mM to about 15 mM of D-alanine and/or 3 to 10 mM of D-serine.

8. The method of claim 1, wherein the promoter active in the barley plant is an ubiquitin promoter.

9. The method of claim 8, wherein selection pressure is applied for 7 to 21 days after co-cultivation.

10. The method of claim 7, wherein the ubiquitin promoter is selected from the group consisting of
a) a sequence comprising the sequence as described by SEQ ID NO: 5, and
b) a sequence comprising at least one fragment of at least 50 consecutive base pairs of the sequence as described by SEQ ID NO: 5, and having promoter activity in barley,
c) a sequence comprising a sequence having at least 60% identity to the sequence as described by SEQ ID NO: 5, and having promoter activity in barley,
d) a sequence comprising a sequence hybridizing under stringent conditions to the sequence as described by SEQ ID NO: 5, wherein stringent conditions are defined by
i. either a combination of conditions comprising
1. salt concentrations of less than about 1.5 M,
2. pH 7.0 to 8.3 and
3. a temperature of at least about 30° C.
ii. or addition of an effective amount of at least one DNA duplex destabilizing agent,
and having promoter activity in barley.

11. The method of claim 8, wherein the ubiquitin promoter is selected from the group consisting of
a) a sequence comprising the sequence as described by SEQ ID NO: 6, and
b) a sequence comprising at least one fragment of at least 50 consecutive base pairs of the sequence as described by SEQ ID NO: 6, and having promoter activity in barley,
c) a sequence comprising a sequence having at least 60% identity to the sequence as described by SEQ ID NO: 6, and having promoter activity in barley,
d) a sequence comprising a sequence hybridizing to the sequence under stringent conditions as described by SEQ ID NO: 6, wherein stringent conditions are defined by
iii. either a combination of conditions comprising
4. salt concentrations of less than about 1.5 M,
5. pH 7.0 to 8.3 and
6. a temperature of at least about 30° C.
iv. or addition of DNA duplex destabilizing agents,
and having promoter activity in barley.

12. The method of claim 1, wherein the selection of step b) is done using 3 to 10 mM D-alanine and/or D-serine.

13. The method of claim 1, wherein the total selection time under dedifferentiating conditions is from 3 to 8 weeks.

14. The method of claim 1, wherein the selection of step b) is done in two steps, using a first selection step for 7 to 35 days, then transferring the surviving cells or tissue to a second selection medium with essentially the same composition than the first selection medium for additional 7 to 35 days.

15. The method of claim 1, wherein introduction of said DNA construct is mediated by a method selected from the group consisting of Rhizobiaceae mediated transformation and particle bombardment mediated transformation.

16. The method of claim 15, wherein the Rhizobiaceae bacterium is a disarmed Agrobacterium tumefaciens or Agrobacterium rhizogenes bacterium.

17. The method of claim 1, wherein the barley plant is selected from the group of Hordeum family.

18. The method of claim 17, wherein the barley cell or tissue or the immature embryo is isolated from a plant species selected from the group consisting of Hordeum vulgare subsp. Vulgare and Hordeum vulgare subsp. Spontaneum.

19. The method of claim 1, wherein said method comprises:
i) transforming a barley plant cell with a first DNA construct comprising
a) at least one first expression construct comprising a promoter active in said barley plant and operably linked thereto a nucleic acid sequence encoding a D-amino acid oxidase enzyme, wherein said first expression cassette is flanked by sequences which allow for specific deletion of said first expression cassette, and
b) at least one second expression cassette suitable for conferring to said plant an agronomically valuable trait, wherein said second expression cassette is not localized between said sequences which allow for specific deletion of said first expression cassette, and
ii) treating said transformed barley plant cells of step i) with a first compound selected from the group consisting of D-alanine, D-serine or derivatives thereof in a phytotoxic concentration and selecting plant cells comprising in their genome said first DNA construct, conferring resistance to said transformed plant cells against said first compound by expression of said D-amino acid oxidase, and
iii) inducing deletion of said first expression cassette from the genome of said transformed plant cells and treating said plant cells with a second compound selected from the group consisting of D-isoleucine, D-valine and derivatives thereof in a concentration toxic to plant cells still comprising said first expression cassette, thereby selecting plant cells comprising said second expression cassette but lacking said first expression cassette.

20. The method of claim 19, wherein
a) the promoter is a ubiquitin promoter and/or
b) the D-amino acid oxidase is selected from the group consisting of
i) the Rhodotorula gracilis D-amino acid oxidase as encoded by SEQ ID NO: 4,
ii) an enzyme having the same enzymatic activity and an identity of at least 80% to the sequence as encoded by SEQ ID NO: 4, and
iii) an enzyme encoded by a nucleic acid sequence capable of hybridizing to the complement of the sequence described by SEQ ID NO: 3.

21. A barley plant or cell produced by the method of claim 1.

22. The barley plant or cell of claim 21, wherein
a) the promoter is a ubiquitin promoter selected from the group consisting of
i) a sequence comprising the sequence as described by SEQ ID NO: 5;
ii) a sequence comprising at least one fragment of at least 50 consecutive base pairs of the sequence as described by SEQ ID NO: 5, and having promoter activity in barley;
iii) a sequence comprising a sequence having at least 60% identity to the sequence as described by SEQ ID NO: 5 and having promoter activity in barley;
iv) a sequence comprising a sequence hybridizing under stringent conditions to the sequence as described by SEQ ID NO: 5, wherein stringent conditions are defined by
1. either a combination of conditions comprising
(a) salt concentrations of less than about 1.5 M,
(b) pH 7.0 to 8.3 and
(c) a temperature of at least about 30° C.
2. or addition of an effective amount of at least one DNA duplex destabilizing agent,
and having promoter activity in barley;
v) a sequence comprising the sequence as described by SEQ ID NO: 6;
vi) a sequence comprising at least one fragment of at least 50 consecutive base pairs of the sequence as described by SEQ ID NO: 6, and having promoter activity in barley;
vii) a sequence comprising a sequence having at least 60% identity to the sequence as described by SEQ ID NO: 6, and having promoter activity in barley; and
viii) a sequence comprising a sequence hybridizing to the sequence under stringent conditions as described by SEQ ID NO: 6, wherein stringent conditions are defined by
1. either a combination of conditions comprising
(a) salt concentrations of less than about 1.5 M,
(b) pH 7.0 to 8.3 and
(c) a temperature of at least about 30° C.
2. or addition of DNA duplex destabilizing agents,
and having promoter activity in barley,
and/or
b) the enzyme capable of metabolizing D-alanine or D-serine is selected from the group consisting of
i) a D-amino acid oxidase (EC 1.4.3.3) whose organism of origin is selected from the group consisting of Caenorhabditis elegans, Cavia parcellus (Guinea pig) Cricetulus griseus, Drosophila melanogaster, Fusarium solani, Fusarium solani (subsp. pisi), Homo sapiens (Human), Mus musculus (Mouse), Mycobacterium leprae, Neisseria meningitides, Neurospora crassa, Oryctolagus cuniculus, Rattus norvegicus (Rat), Rhodosporidium toruloides, Schizosaccharomyces pombe (Fission yeast), Streptomyces avermitilis, Streptomyces coelicolor, Sus scrofa (Pig), Trigonopsis variabilis, Xanthomonas axonopodis and Xanthomonas campestris;
ii) the E. coli D-serine ammonia-lyase as encoded by SEQ ID NO: 2;
iii) an enzyme having the same enzymatic activity and an identity of at least 80% to the sequence as encoded by SEQ ID NO: 2;
iv) an enzyme encoded by a nucleic acid sequence capable of hybridizing under stringent conditions to the complement of the sequence described by SEQ ID NO: 1, wherein stringent conditions are defined by
1. either a combination of conditions comprising
(a) salt concentrations of less than about 1.5 M,
(b) pH 7.0 to 8.3 and
(c) a temperature of at least about 30° C.,
2. or addition of an effective amount of at least one DNA duplex destabilizing agent;
v) the Rhodotorula gracilis D-amino acid oxidase as encoded by SEQ ID NO: 4;
vi) an enzyme having the same enzymatic activity and an identity of at least 80% to the sequence as encoded by SEQ ID NO: 4; and
vii) an enzyme encoded by a nucleic acid sequence capable of hybridizing to the complement of the sequence described by SEQ ID NO: 3.

23. The barley plant or cell of claim 21, further comprising at least one second expression construct conferring to the barley plant an agronomically valuable trait.

24. The barley plant or cell of claim 21, wherein the barley plant is from the Hordeum family.

25. The barley plant or cell of claim 21, wherein said plant or cell is selected from the group consisting of Hordeum vulgare subsp. vulgare and Hordeum vulgare subsp. spontaneum.

26. A part of the barley plant of claim 21 comprising a promoter active in said barley plant or cells and operably linked thereto a nucleic acid sequence encoding an enzyme capable metabolizing D-alanine or D-serine, wherein the promoter is heterologous in relation to the enzyme encoding sequence.

27. A method for subsequent transformation of at least two DNA constructs into a barley plant comprising:
a) a transformation with a first construct said construct comprising at least one expression construct comprising a promoter active in a barley plant and operably linked thereto a nucleic acid sequence encoding an enzyme capable of metabolizing D-alanine or D-serine, and
b) a transformation with a second construct said construct comprising a second selection marker gene, which is not conferring resistance against D-alanine or D-serine.

28. The method of claim 27, wherein said second marker gene is conferring resistance against at least one compound selected from the group consisting of phosphinotricin, glyphosate, sulfonylurea- and imidazolinone-type herbicides.

29. A barley plant comprising
a) a first expression construct comprising a promoter active in a barley plant and operably linked thereto a nucleic acid sequence encoding an enzyme capable of metabolizing D-alanine or D-serine, and
b) a second expression construct for a selection marker gene, which is not conferring resistance against D-alanine or D-serine and comprises one or more of the following:
i. Bromoxynil® degrading nitrilases or
ii. dihydrofolate reductase
and/or confers resistance to 2-deoxyglucose-6-phosphate.

30. A method for subsequent transformation of at least two DNA constructs into a barley plant comprising:
a) a transformation with a first construct said construct comprising an expression construct comprising a promoter active in a barley plant and operably linked thereto a nucleic acid sequence encoding an D-serine dehydratase enzyme and selecting with D-serine, and
b) a transformation with a second construct said construct comprising an expression construct comprising promoter active in a barley plant and operably linked thereto a nucleic acid sequence encoding a D-amino acid oxidase enzyme and selecting with D-alanine.

31. A barley plant comprising
a) a first construct said construct comprising an expression construct comprising a promoter active in a barley plant and operably linked thereto a nucleic acid sequence encoding an D-serine dehydratase enzyme, and
b) a second construct said construct comprising an expression construct comprising promoter active in a barley plant and operably linked thereto a nucleic acid sequence encoding a D-amino acid oxidase enzyme.

32. A composition for selection, regeneration, growing, cultivation or maintaining of a transgenic barley plant cell, a transgenic barley plant tissue, a transgenic barley plant organ or a transgenic barley plant or a part thereof comprising
a. L-proline or
b. glutamine
together with an effective amount of D-alanine, D-serine, or a derivative thereof allowing for selection of transgenic barley plant cells, barley plant tissue, barley plant organs or barley plants or a part thereof and a transgenic barley organism, a transgenic barley cell, a transgenic cell culture, a transgenic barley plant and/or a part thereof.

33. A cell culture comprising one or more embryogenic calli derived from immature barley embryo(s), and an effective amount of at least one auxin, wherein the effective amount of the auxin compound is equivalent to a concentration of 0.2 mg/l to 6 mg/l 2,4-D, and D-alanine and/or D-serine in a total concentration from 1 mM to about 15 mM of D-alanine and/or 3 to 10 mM of D-serine.

34. A recovery medium for barley plants or barley tissues comprising an effective amount of at least one antibiotic that inhibits or suppresses the growth of the soil-borne bacteria, and L-proline in a concentration from 0.5 g/l to 2 g/l.

35. A selection medium comprising D-alanine and/or D-serine or a derivative thereof in a phytotoxic concentration of 1 mM to about 15 mM of D-alanine and/or 3 to 10 mM of D-serine.

36. A regeneration medium comprising a concentration of 1 mM to about 15 mM of D-alanine and/or 3 to 10 mM of D-serine, or a derivative thereof allowing for selection of transgenic cells, and one or more compounds selected from the group consisting of:
i) cytokinins in a concentration from 0.5 to 10 mg/L,
ii) an effective amount of at least one antibiotic that inhibits or suppresses the growth of the soil-borne bacteria, and

37. A method for selection of transgenic barley plants and cells comprising a medium comprising a concentration of 1 mM to about 15 mM of D-alanine and/or 3 to 10 mM of D-serine or a derivative thereof allowing for selection of transgenic cells.

38. The method according to claim 37, wherein the method comprises the use of a plant growth regulator.

39. The method according to claim 37, wherein the method comprises the use of
a. L-proline or
b. glutamine.

40. The method of claim 2, wherein the T-DNA further comprises at least one second expression construct conferring to the barley plant an agronomically valuable trait.

41. The method of claim 8, wherein the ubiquitin promoter is a maize ubiquitin promoter.


SEQUENCE LISTING (29 sequences)

SEQ ID NO: 1 - DNA, 1329 bp, Escherichia coli
  CDS (1)..(1329): E.coli D-serine dehydratase [dsdA] gene/CDS
atggaaaacgctaaaatgaactcgctcatcgcccagtatccgttggtaaaggatctggttgctcttaaagaaaccacctggtttaatcctggcacgacctcattggctgaaggtttaccttatgttggcctgaccgaacaggatgttcaggacgcccatgcgcgcttatcccgttttgcaccctatctggcaaaagcatttcctgaaactgctgccactggggggattattgaatcagaactggttgccattccagctatgcaaaaacggctggaaaaagaatatcagcaaccgatcagcgggcaactgttactgaaaaaagatagccatttgcccatttccggctccataaaagcacgcggcgggatttatgaagtcctggcacacgcagaaaaactggctctggaagcggggttgctgacgcttgatgatgactacagcaaactgctttctccggagtttaaacagttctttagccaatacagcattgctgtgggctcaaccggaaatctggggttatcaatcggcattatgagcgcccgcattggctttaaggtgacagttcatatgtctgctgatgcccgggcatggaaaaaagcgaaactgcgcagccatggcgttacggtcgtggaatatgagcaagattatggtgttgccgtcgaggaaggacgtaaagcagcgcagtctgacccgaactgtttctttattgatgacgaaaattcccgcacgttgttccttgggtattccgtcgctggccagcgtcttaaagcgcaatttgcccagcaaggccgtatcgtcgatgctgataaccctctgtttgtctatctgccgtgtggtgttggcggtggtcctggtggcgtcgcattcgggcttaaactggcgtttggcgatcatgttcactgcttttttgccgaaccaacgcactccccttgtatgttgttaggcgtccatacaggattacacgatcagatttctgttcaggatattggtatcgacaaccttaccgcagcggatggccttgcagttggtcgcgcatcaggctttgtcgggcgggcaatggagcgtctgctggatggcttctatacccttagcgatcaaaccatgtatgacatgcttggctggctggcgcaggaagaaggtattcgtcttgaaccttcggcactggcgggtatggccggacctcagcgcgtgtgtgcatcagtaagttaccaacagatgcacggtttcagcgcagaacaactgcgtaataccactcatctggtgtgggcgacgggaggtggaatggtgccggaagaagagatgaatcaatatctggcaaaaggccgttaa

SEQ ID NO: 2 - protein, 442 aa, Escherichia coli
MENAKMNSLIAQYPLVKDLVALKETTWFNPGTTSLAEGLPYVGLTEQDVQDAHARLSRFAPYLAKAFPETAATGGIIESELVAIPAMQKRLEKEYQQPISGQLLLKKDSHLPISGSIKARGGIYEVLAHAEKLALEAGLLTLDDDYSKLLSPEFKQFFSQYSIAVGSTGNLGLSIGIMSARIGFKVTVHMSADARAWKKAKLRSHGVTVVEYEQDYGVAVEEGRKAAQSDPNCFFIDDENSRTLFLGYSVAGQRLKAQFAQQGRIVDADNPLFVYLPCGVGGGPGGVAFGLKLAFGDHVHCFFAEPTHSPCMLLGVHTGLHDQISVQDIGIDNLTAADGLAVGRASGFVGRAMERLLDGFYTLSDQTMYDMLGWLAQEEGIRLEPSALAGMAGPQRVCASVSYQQMHGFSAEQLRNTTHLVWATGGGMVPEEEMNQYLAKGR

SEQ ID NO: 3 - DNA, 1107 bp, Rhodosporidium toruloides
  CDS (1)..(1107): Rhodosporidium toruloides D-amino acid oxidase CDS
atgcactcgcagaagcgcgtcgttgtcctcggatcaggcgttatcggtctgagcagcgccctcatcctcgctcggaagggctacagcgtgcatattctcgcgcgcgacttgccggaggacgtctcgagccagactttcgcttcaccatgggctggcgcgaattggacgcctttcatgacgcttacagacggtcctcgacaagcaaaatgggaagaatcgactttcaagaagtgggtcgagttggtcccgacgggccatgccatgtggctcaaggggacgaggcggttcgcgcagaacgaagacggcttgctcgggcactggtacaaggacatcacgccaaattaccgccccctcccatcttccgaatgtccacctggcgctatcggcgtaacctacgacaccctctccgtccacgcaccaaagtactgccagtaccttgcaagagagctgcagaagctcggcgcgacgtttgagagacggaccgttacgtcgcttgagcaggcgttcgacggtgcggatttggtggtcaacgctacgggacttggcgccaagtcgattgcgggcatcgacgaccaagccgccgagccaatccgcgggcaaaccgtcctcgtcaagtccccatgcaagcgatgcacgatggactcgtccgaccccgcttctcccgcctacatcattccccgaccaggtggcgaagtcatctgcggcgggacgtacggcgtgggagactgggacttgtctgtcaacccagagacggtccagcggatcctcaagcactgcttgcgcctcgacccgaccatctcgagcgacggaacgatcgaaggcatcgaggtcctccgccacaacgtcggcttgcgacctgcacgacgaggcggaccccgcgttgaggcagaacggatcgtcctgcctctcgaccggacaaagtcgcccctctcgctcggcaggggcagcgcacgagcggcgaaggagaaggaggtcacgcttgtgcatgcgtatggcttctcgagtgcgggataccagcagagttggggcgcggcggaggatgtcgcgcagctcgtcgacgaggcgttccagcggtaccacggcgcggcgcgggagtcgaagttgtag

SEQ ID NO: 4 - protein, 368 aa, Rhodosporidium toruloides
MHSQKRVVVLGSGVIGLSSALILARKGYSVHILARDLPEDVSSQTFASPWAGANWTPFMTLTDGPRQAKWEESTFKKWVELVPTGHAMWLKGTRRFAQNEDGLLGHWYKDITPNYRPLPSSECPPGAIGVTYDTLSVHAPKYCQYLARELQKLGATFERRTVTSLEQAFDGADLVVNATGLGAKSIAGIDDQAAEPIRGQTVLVKSPCKRCTMDSSDPASPAYIIPRPGGEVICGGTYGVGDWDLSVNPETVQRILKHCLRLDPTISSDGTIEGIEVLRHNVGLRPARRGGPRVEAERIVLPLDRTKSPLSLGRGSARAAKEKEVTLVHAYGFSSAGYQQSWGAAEDVAQLVDEAFQRYHGAARESKL

SEQ ID NO: 5 - DNA, 275 bp, Zea mays
  promoter (1)..(275): Zea mays ubiquitin 275 bp core promoter
aaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctct

SEQ ID NO: 6 - DNA, 1988 bp, Zea mays
  promoter (1)..(1988): Zea mays ubiquitin promoter region with 5′UTR and intron
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttactt

SEQ ID NO: 7 - DNA, 275 bp, Sugarcane bacilliform virus
  promoter (1)..(275): Sugarcane bacilliform virus 275 bp core promoter
gattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

SEQ ID NO: 8 - DNA, 1398 bp, Sugarcane bacilliform virus
  promoter (1)..(1398): Sugarcane bacilliform virus [ScBV] promoter region
aatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccaag

SEQ ID NO: 9 - DNA, 5035 bp, Artificial
  Nucleic acid sequence encoding pRLM175, a kanamycin resistant pSB11-type binary vector.
agtggtgattttgtgccgagctgccggtcggggagctgttggctggctggtggcaggatatattgtggtgtaaacaaattgacgcttagacaacttaataacacattgcggacgtttttaatgtactgaattggatccgcccgggcggtaccgaattcgcggccgcaagcttgtacaacgcgtaccggttaattaatctagaggcgcgccgggcccggccggccagatcttgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatcgtcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccgggggggggggggggggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaccccccccccccccccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcccccgatcaagtagatacactacatatatctacaatagacatcgagccggaaggtgatgtttactttcctgaaatccccagcaattttaggccagtttttacccaagacttcgcctctaacataaattatagttaccaaatctggcaaaagggttgaccggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcagggatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcg

SEQ ID NO: 10 - DNA, 8269 bp, Artificial
  Nucleic acid sequence encoding T-DNA region of pRLM166, a pRLM175 derived binary vector containing p-ZmUBI+I::c-dsdA::t-OCS and p-ScBV::c-gusINT::t-NOS cassettes.
gtgattttgtgccgagctgccggtcggggagctgttggctggctggtggcaggatatattgtggtgtaaacaaattgacgcttagacaacttaataacacattgcggacgtttttaatgtactgaattggatccgcccgggcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatcctcatctaagcgcaaagagacgtactatggaaaacgctaaaatgaactcgctcatcgcccagtatccgttggtaaaggatctggttgctcttaaagaaaccacctggtttaatcctggcacgacctcattggctgaaggtttaccttatgttggcctgaccgaacaggatgttcaggacgcccatgcgcgcttatcccgttttgcaccctatctggcaaaagcatttcctgaaactgctgccactggggggattattgaatcagaactggttgccattccagctatgcaaaaacggctggaaaaagaatatcagcaaccgatcagcgggcaactgttactgaaaaaagatagccatttgcccatttccggctccataaaagcacgcggcgggatttatgaagtcctggcacacgcagaaaaactggctctggaagcggggttgctgacgcttgatgatgactacagcaaactgctttctccggagtttaaacagttctttagccaatacagcattgctgtgggctcaaccggaaatctggggttatcaatcggcattatgagcgcccgcattggctttaaggtgacagttcatatgtctgctgatgcccgggcatggaaaaaagcgaaactgcgcagccatggcgttacggtcgtggaatatgagcaagattatggtgttgccgtcgaggaaggacgtaaagcagcgcagtctgacccgaactgtttctttattgatgacgaaaattcccgcacgttgttccttgggtattccgtcgctggccagcgtcttaaagcgcaatttgcccagcaaggccgtatcgtcgatgctgataaccctctgtttgtctatctgccgtgtggtgttggcggtggtcctggtggcgtcgcattcgggcttaaactggcgtttggcgatcatgttcactgcttttttgccgaaccaacgcactccccttgtatgttgttaggcgtccatacaggattacacgatcagatttctgttcaggatattggtatcgacaaccttaccgcagcggatggccttgcagttggtcgcgcatcaggctttgtcgggcgggcaatggagcgtctgctggatggcttctatacccttagcgatcaaaccatgtatgacatgcttggctggctggcgcaggaagaaggtattcgtcttgaaccttcggcactggcgggtatggccggacctcagcgcgtgtgtgcatcagtaagttaccaacagatgcacggtttcagcgcagaacaactgcgtaataccactcatctggtgtgggcgacgggaggtggaatggtgccggaagaagagatgaatcaatatctggcaaaaggccgttaataacgtttcaacgcagcatggatcgtaccgagctcaatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatgggcccaggtacagaattcgcggccgtacaacgcgtaccggttaattaaggtacccaattgcatatgtaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccaagggggtacgatctccgggtaggtcagtcccttatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgggcccggccggccagatcttgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgt

SEQ ID NO: 11 - DNA, 7838 bp, Artificial
  Nucleic acid sequence encoding T-DNA region of pRLM167, a pRLM175 derived binary vector containing p-ZmUBI+I::c-dsdA::t-OCS and p-ZmUBI+I::c-PAT::t-OCS cassettes.
gtgattttgtgccgagctgccggtcggggagctgttggctggctggtggcaggatatattgtggtgtaaacaaattgacgcttagacaacttaataacacattgcggacgtttttaatgtactgaattggatccgcccgggcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatcctcatctaagcgcaaagagacgtactatggaaaacgctaaaatgaactcgctcatcgcccagtatccgttggtaaaggatctggttgctcttaaagaaaccacctggtttaatcctggcacgacctcattggctgaaggtttaccttatgttggcctgaccgaacaggatgttcaggacgcccatgcgcgcttatcccgttttgcaccctatctggcaaaagcatttcctgaaactgctgccactggggggattattgaatcagaactggttgccattccagctatgcaaaaacggctggaaaaagaatatcagcaaccgatcagcgggcaactgttactgaaaaaagatagccatttgcccatttccggctccataaaagcacgcggcgggatttatgaagtcctggcacacgcagaaaaactggctctggaagcggggttgctgacgcttgatgatgactacagcaaactgctttctccggagtttaaacagttctttagccaatacagcattgctgtgggctcaaccggaaatctggggttatcaatcggcattatgagcgcccgcattggctttaaggtgacagttcatatgtctgctgatgcccgggcatggaaaaaagcgaaactgcgcagccatggcgttacggtcgtggaatatgagcaagattatggtgttgccgtcgaggaaggacgtaaagcagcgcagtctgacccgaactgtttctttattgatgacgaaaattcccgcacgttgttccttgggtattccgtcgctggccagcgtcttaaagcgcaatttgcccagcaaggccgtatcgtcgatgctgataaccctctgtttgtctatctgccgtgtggtgttggcggtggtcctggtggcgtcgcattcgggcttaaactggcgtttggcgatcatgttcactgcttttttgccgaaccaacgcactccccttgtatgttgttaggcgtccatacaggattacacgatcagatttctgttcaggatattggtatcgacaaccttaccgcagcggatggccttgcagttggtcgcgcatcaggctttgtcgggcgggcaatggagcgtctgctggatggcttctatacccttagcgatcaaaccatgtatgacatgcttggctggctggcgcaggaagaaggtattcgtcttgaaccttcggcactggcgggtatggccggacctcagcgcgtgtgtgcatcagtaagttaccaacagatgcacggtttcagcgcagaacaactgcgtaataccactcatctggtgtgggcgacgggaggtggaatggtgccggaagaagagatgaatcaatatctggcaaaaggccgttaataacgtttcaacgcagcatggatcgtaccgagctcaatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatgggcccaggtacagaattcgcggccgtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatcctctagagtcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgacctgcaggagctcaatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatgggcccaggtacagaattcgcggccgcttaattaatctagaggcgcgccgggcccggccggccagatcttgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgt

SEQ ID NO: 12 - DNA, 8017 bp, Artificial
  Nucleic acid sequence encoding T-DNA region of pRLM205, a pRLM175 derived binary vector containing p-ZmUBI+I::c-dao1::t-OCS and p-ScBV::c-gusINT::t-NOS cassettes.
gtgattttgtgccgagctgccggtcggggagctgttggctggctggtggcaggatatattgtggtgtaaacaaattgacgcttagacaacttaataacacattgcggacgtttttaatgtactgaattggatccgcccgggcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatccggcgcgccactagtcccgggccaccatgcactcgcagaagcgcgtcgttgtcctcggatcaggcgttatcggtctgagcagcgccctcatcctcgctcggaagggctacagcgtgcatattctcgcgcgcgacttgccggaggacgtctcgagccagactttcgcttcaccatgggctggcgcgaattggacgcctttcatgacgcttacagacggtcctcgacaagcaaaatgggaagaatcgactttcaagaagtgggtcgagttggtcccgacgggccatgccatgtggctcaaggggacgaggcggttcgcgcagaacgaagacggcttgctcgggcactggtacaaggacatcacgccaaattaccgccccctcccatcttccgaatgtccacctggcgctatcggcgtaacctacgacaccctctccgtccacgcaccaaagtactgccagtaccttgcaagagagctgcagaagctcggcgcgacgtttgagagacggaccgttacgtcgcttgagcaggcgttcgacggtgcggatttggtggtcaacgctacgggacttggcgccaagtcgattgcgggcatcgacgaccaagccgccgagccaatccgcgggcaaaccgtcctcgtcaagtccccatgcaagcgatgcacgatggactcgtccgaccccgcttctcccgcctacatcattccccgaccaggtggcgaagtcatctgcggcgggacgtacggcgtgggagactgggacttgtctgtcaacccagagacggtccagcggatcctcaagcactgcttgcgcctcgacccgaccatctcgagcgacggaacgatcgaaggcatcgaggtcctccgccacaacgtcggcttgcgacctgcacgacgaggcggaccccgcgttgaggcagaacggatcgtcctgcctctcgaccggacaaagtcgcccctctcgctcggcaggggcagcgcacgagcggcgaaggagaaggaggtcacgcttgtgcatgcgtatggcttctcgagtgcgggataccagcagagttggggcgcggcggaggatgtcgcgcagctcgtcgacgaggcgttccagcggtaccacggcgcggcgcgggagtcgaagttgtagggcgggattcctgcaggagctcaatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatgggcccaggtacagaattcgcggccgcttaattaaggtacccaattgcatatgtaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccaagggggtacgatctccgggtaggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgggcccggccggccagatcttgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgt

SEQ ID NO: 13 - DNA, 23 bp, Artificial
  Nucleic acid sequence encoding qPCR primer GUSCommon-341F
ccgggtgaaggttatctctatga

SEQ ID NO: 14 - DNA, 24 bp, Artificial
  Nucleic acid sequence encoding qPCR primer GUSCommon-414R
cgaagcgggtagatatcacactct

SEQ ID NO: 15 - DNA, 24 bp, Artificial
  Nucleic acid sequence encoding qPCR probe GUSCommon-366FAM
tgtgcgtcacagccaaaagccaga

SEQ ID NO: 16 - DNA, 20 bp, Artificial
  Nucleic acid sequence encoding qPCR primer EcdsdA-860F
tcgcattcgggcttaaactg

SEQ ID NO: 17 - DNA, 18 bp, Artificial
  Nucleic acid sequence encoding qPCR primer EcdsdA-922R
gcgttggttcggcaaaaa

SEQ ID NO: 18 - DNA, 21 bp, Artificial
  Nucleic acid sequence encoding qPCR probe EcdsdA-883FAM
tttggcgatcatgttcactgc

SEQ ID NO: 19 - DNA, 19 bp, Artificial
  Nucleic acid sequence encoding qPCR primer dao1/pa-285F
gttcgcgcagaacgaagac

SEQ ID NO: 20 - DNA, 18 bp, Artificial
  Nucleic acid sequence encoding qPCR primer dao1/pa-349R
ggcggtaatttggcgtga

SEQ ID NO: 21 - DNA, 22 bp, Artificial
  Nucleic acid sequence encoding qPCR probe dao1/pa-308FAM
tccttgtaccagtgcccgagca

SEQ ID NO: 22 - DNA, 20 bp, Artificial
  Nucleic acid sequence encoding forward PCR primer for gusINT gene
accgtttgtgtgaacaacga

SEQ ID NO: 23 - DNA, 20 bp, Artificial
  Nucleic acid sequence encoding reverse PCR primer for gusINT gene
ggcacagcacatcaaagaga

SEQ ID NO: 24 - DNA, 22 bp, Artificial
  Nucleic acid sequence encoding forward PCR primer for dsdA gene
gctttttgttcgcttggttgtg

SEQ ID NO: 25 - DNA, 20 bp, Artificial
  Nucleic acid sequence encoding reverse PCR primer for dsdA gene
tcaataatccccccagtggc

SEQ ID NO: 26 - DNA, 22 bp, Artificial
  Nucleic acid sequence encoding forward PCR primer for dao1 gene
gacaagcaaaatgggaagaatc

SEQ ID NO: 27 - DNA, 18 bp, Artificial
  Nucleic acid sequence encoding reverse PCR primer for dao1 gene
tcggggaatgatgtaggc

SEQ ID NO: 28 - DNA, 29 bp, Artificial
  Nucleic acid sequence encoding forward PCR primer for PAT gene
atgtctccggagaggagaccagttgagat

SEQ ID NO: 29 - DNA, 26 bp, Artificial
  Nucleic acid sequence encoding reverse PCR primer for PAT gene
gccaaaaaccaacatcatgccatcca